(19) United States
(12) Patent Application Publication
(10) Pub. No.: US 2024/0368092 A1
(43) Pub. Date: Nov. 7, 2024

(54) 2,4-Dihydro-3H-1,2,4-Triazol-3-One P2X7 Antagonists

(71) Applicant: Breye Therapeutics ApS, Hørsholm (DK)

(72) Inventors: Maria Pia CATALANI, Bresso (IT); Paolo PEVARELLO, Bresso (IT); Mariangela SODANO, Bresso (IT)

(73) Assignee: Breye Therapeutics ApS, Hørsholm (DK)

(21) Appl. No.: 18/683,006

(22) PCT Filed: Sep. 1, 2021
(86) PCT No.: PCT/EP2022/074293

§ 371 Date: Feb. 12, 2024

(30) Foreign Application Priority Data
Sep. 3, 2021 (EP) 21194819.5

(51) Int. Cl. C07D 249/12; A61K 31/4196; A61K 31/4439; A61K 31/501; A61K 31/5377; C07D 401/06; C07D 401/10; C07D 405/06; C07D 413/06

(52) U.S. Cl. CPC C07D 249/12; A61K 31/4196; A61K 31/4439; A61K 31/501; A61K 31/5377; C07D 401/06; C07D 401/10; C07D 405/06; C07D 413/06

(57) ABSTRACT

The present invention relates to novel 2,4-dihydro-3H-1,2,4-triazol-3-one compounds of formula (I) having P2X7 receptor (P2X7) antagonistic properties, pharmaceutical compositions comprising these compounds, chemical processes for preparing these compounds and their use in the treatment or prophylaxis of diseases associated with P2X7 receptor activity in animals, in particular humans.

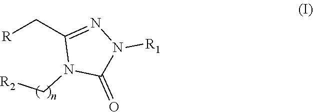

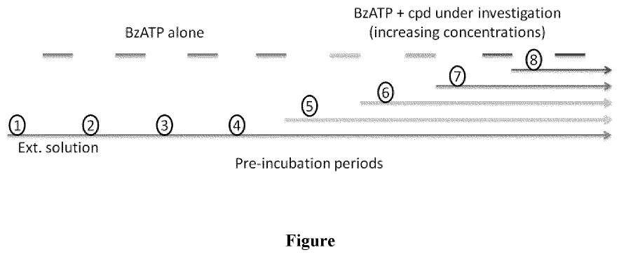

The present invention relates to novel 2,4-dihydro-3H-1,2,4-triazol-3-one compounds of formula (I) having P2X7 receptor (P2X7) antagonistic properties, pharmaceutical compositions comprising these compounds, chemical processes for preparing these compounds and their use in the treatment or prophylaxis of diseases associated with P2X7 receptor activity in animals, in particular humans.

P2X7 belongs to the family of P2X ionotropic receptors. P2X7 is activated by extracellular nucleotides, notably adenosine triphosphate (ATP). P2X7 is distinguished from other P2X family members by the specific localization (CNS and immunocompetent cells in particular), by the high concentrations of ATP (in the mM range) required to activate it and by its ability to form a large pore upon prolonged or repeated stimulation. P2X7 is a ligand-gated ion channel and is present on a variety of cell types, largely those known to be involved in the inflammatory and/or immune process, specifically, macrophages, mast cells and lymphocytes (T and B). Activation of the P2X7 receptor by extracellular nucleotides, e.g., ATP, leads to the release of interleukin-1β (1L-1β) and giant cell formation (macrophages/microglial cells), degranulation (mast cells) and L-selectin shedding (lymphocytes). P2X7 receptors are also located on antigen-presenting cells (APC), keratinocytes, salivary acinar cells (parotid cells), hepatocytes, erythrocytes, erythroleukaemic cells, monocytes, fibroblasts, bone marrow cells, neurones, and renal mesangial cells. The P2X7 receptor is also known to be a pain sensor in the nervous system. Experiments using P2X7 deficient mice demonstrate the role of P2X7 in the development of pain as these mice were protected from the development of both adjuvant-induced inflammatory pain and partial nerve ligation induced neuropathic pain. There is also growing evidence that P2X7 or its downstream effectors, such as IL-1β, are involved in the pathophysiology of several neurological disorders, such as, Alzheimer's Disease (J. I. Diaz-Hernandez et al., Neurobiol. Aging 2012, 1816-1828: In vivo P2X7 inhibition reduces Aβ plaques in AD through GSK3β). P2X7 is thought to have an important function in neurotransmission within the CNS through its activation on postsynaptic and/or presynaptic neurons and glia. Data has emerged using in situ hybridization that P2X7 receptor mRNA is widely distributed throughout the rat brain. Specifically, areas of high P2X7 mRNA expression were found in the anterior olfactory nucleus, cerebral cortex, piriform cortex (Pir), lateral septal nucleus (LS), hippocampal pyramidal cell layers of CA1, CA3, CA4, pontine nuclei, external cuneate nucleus, and medial vestibular nucleus. P2X7 hybridization signals were also observed in the motor neurons of the trigeminal motor nucleus, facial nucleus, hypoglossal nucleus, and the anterior horn of the spinal cord.

Hence there is a therapeutic rationale for the use of P2X7 antagonists in the treatment of a variety of disease states. These states include but are not limited to diseases associated with the CNS such as Alzheimer's Disease, Parkinson's Disease, Huntington's Disease, Amyotrophic Lateral Sclerosis, spinal cord injury, cerebral ischemia, head trauma, meningitis, sleep disorders, mood and anxiety disorders, HIV-induced neuroinflammation, and chronic neuropathic and inflammatory pain. Furthermore, peripheral inflammatory disorders and autoimmune diseases including but not limited to rheumatoid arthritis, ostheoarthritis, psoriasis, allergic dermatitis, asthma, chronic obstructive pulmonary disease, airways hyper-responsiveness, septic shock, bronchitis, glomerulonephritis, irritable bowel syndrome, fatty liver disease, liver fibrosis, skin injury, lung emphysema, muscular dystrophy, fibrosis, atherosclerosis, burn injury, Crohn's Disease, ulcerative colitis, age-related macular degeneration, growth and metastasis of malignant cells, Sjögren's syndrome, myoblastic leukaemia, diabetes, osteoporosis, ischemic heart disease are all examples where the involvement of P2X7 receptors has been implicated. In view of the clinical importance of P2X7, the identification of compounds that modulate P2X7 receptor function represents an attractive avenue into the development of new therapeutic agents.

P2X7 inhibitors are described in various patent applications:

WO2004099146 discloses benzamide inhibitors of the P2X7 receptor and their use in the treatment of inflammatory diseases.

WO2009108551 discloses heteroarylamide analogs and their use in P2X7 receptor mediated conditions.

WO2009132000 discloses quinoline and isoquinoline substituted P2X7 receptor antagonists and their use in P2X7 receptor mediated conditions.

WO2015119018 discloses thiazole and oxazole derivatives as P2X7 receptor antagonists and their use in P2X7 receptor mediated conditions.

WO2015/099107 A1 discloses pyrimidinone substituted P2X7 receptor antagonists and their use in P2X7 receptor mediated conditions.

WO2018202694A1 discloses oxadiazolinone compounds P2X7 receptor antagonists and their use in P2X7 receptor mediated conditions.

However there is still an unmet need for compounds which are able to efficiently antagonize P2X7 and that can be delivered in the different target organs which are sites of a P2X7 mediated pathology, including the brain. Such compounds are provided herein.

Various embodiments of the invention are presented hereafter.

The present invention relates to 2,4-dihydro-3H-1,2,4-triazol-3-one compounds of the following formula (I) or a pharmaceutically acceptable salt thereof:

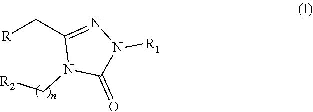

including any stereochemically isomeric form thereof, wherein:
R is an aromatic, aliphatic, heteroaromatic or heteroaliphatic
ring optionally substituted by one or more substituents selected from:
halogen;
C₁-C₄ alkyl optionally substituted by one or more halogen;
R₁ is C₃-C₆ cycloalkyl optionally substituted by one or more halogens or C₁-C₄ alkyl optionally substituted by one or more substituents selected from:
halogen;
OR₃, wherein R₃ is H or C₁-C₄ alkyl optionally substituted by one or more halogens;
NR₄R₅ group, wherein R₄ and R₅ are H or C₁-C₄ alkyl optionally substituted by one or more halogens;
C₃-C₆ cycloalkyl optionally substituted by one or more halogens;
phenyl ring, optionally substituted by halogen;
n is 1 or 2; preferably n is 1;
R₂ is selected from an aromatic, heteroaromatic, aliphatic and heteroaliphatic monocyclic or bicyclic ring optionally substituted by one or more substituents selected from:
halogen;
a C₁-C₄ alkyl optionally substituted by one or more halogens; and
an heteroaromatic ring optionally substituted by one or more halogens;
or R₂ is —OH.

As used in the foregoing definitions:

The terms “halo”, “halogen” and “halide”, which may be used interchangeably, refer to a substituent fluoro, chloro, bromo, or iodo.

The term “stereochemically isomeric forms” as used hereinbefore defines all the possible isomeric forms which the compounds of formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis- or trans-configuration.

Stereochemically isomeric forms of the compounds of formula (I) are obviously intended to be embraced within the scope of this invention.

The absolute stereochemical configuration of the compounds of formula (I) and of the intermediates used in their preparation may easily be determined by those skilled in the art while using well-known methods such as, for example, X-ray diffraction.

Furthermore, some compounds of formula (I) and some of the intermediates used in their preparation may exhibit polymorphism. It is to be understood that the present invention encompasses any polymorphic forms possessing properties useful in the treatment of the conditions noted hereinabove.

The pharmaceutically acceptable salts as mentioned herein above are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, trifluoromethanesulfonic, ethanesulfonic, benzenesulfonic, p-toluenesulfonic, cyclamic, salicylic, p-aminosalicylic, pamoic and the like.

Conversely said salt forms can be converted by treatment with an appropriate base into the free base form.

The compounds of formula (I) may exist in both unsolvated and solvated forms. The term ‘solvate’ is used herein to describe a molecular association comprising a compound of the invention and one or more pharmaceutically acceptable solvent molecules, e.g. water or ethanol. The term ‘hydrate’ is used when said solvent is water.

Preferably, R is phenyl, pyridinyl, cyclohexyl, cycloheptyl, piperidinyl or piperazinyl optionally substituted by one or more substituents selected from:
halogen, preferably Cl and F;
C₁-C₄ alkyl preferably methyl optionally substituted by one or more halogen, preferably methyl or trifluoromethyl.

n is 1 or 2; preferably n is 1.

Preferably, R₁ is C₁-C₄ alkyl, preferably methyl or ethyl, optionally substituted by:
one or more halogens, preferably F,
C₃-C₄ cycloalkyl, preferably cyclopropyl, optionally substituted by halogen, preferably F, or
a phenyl ring.

Preferably, R₂ is selected from aromatic, aliphatic, heteroaromatic or heteroaliphatic ring selected from phenyl, C₃-C₇ cycloakyl, C₆-C₈ bicycloalkanyl, pyridinyl, piperidinyl, tetrahydrofuranyl and morpholinyl, wherein said ring is optionally substituted by one or more substituents selected from halogen, heteroaromatic ring optionally substituted by halogen, and C₁-C₄ alkyl, optionally substituted by one or more halogen atom(s) or R₂ is —OH.

A preferred embodiment of the invention relates to compounds of Formula (I) as defined above wherein:
R is selected from cycloheptyl, cyclohexyl or phenyl, optionally substituted by one or more halogens, preferably F or Cl, C₁-C₄ alkyl, preferably methyl, optionally substituted by halogen, preferably F;
R₁ is selected from C₁-C₄ alkyl, preferably methyl or ethyl, optionally substituted by one or more halogens, preferably F; C₃-C₄ cycloalkyl, preferably cyclopropyl, optionally substituted by halogen, preferably F, or a phenyl ring;
n is 1 or 2; preferably n is 1.

R₂ is selected from:
phenyl, optionally substituted by one or more substituents selected from halogen, preferably F or Cl, C₁-C₄ alkyl optionally substituted by one or more halogen, preferably F, and pyrimidinyl optionally substituted by one or more halogen, preferably F;
pyridinyl optionally substituted by one or more C₁-C₄ alkyl, preferably methyl, optionally substituted by one or more halogen, preferably F;
piperidinyl optionally substituted by C₁-C₄ alkyl, preferably methyl;
cyclohexyl optionally substituted by one or more substituents selected from halogen, preferably F, C₁-C₄ alkyl, preferably methyl, optionally substituted by halogen, preferably F;
cyclopropyl, cyclopentyl or cycloheptyl, optionally substituted by one or more halogen, preferably F;
terahydrofuranyl;
morpholinyl optionally substituted by one or more C₁-C₄ alkyl, preferably methyl;
bicyclo[3.1.0]hexan-3-yl optionally substituted by one or more halogens, preferably F;
spiro[2.5]octan-6-yl; and
—OH

Another preferred embodiment of the invention relates to compounds of Formula (I) as defined above wherein:
R is selected from cyclohexyl, 4,4-difluoro cyclohexyl, cicloheptyl, 2-chloro-6-fluorophenyl, 2-chloro-4-fluorophenyl, 2-fluorophenyl, 4-fluorophenyl, 2-chlorophenyl, 2-trifluoromethylphenyl.

R₁ is selected from hydrogen, methyl, ethyl, 2,2,2-trifluoroethyl, cyclopropylmethyl, benzyl, 3,3,3-trifluoropropyl, 2,2-difluoroethyl.

n is 1 or 2; preferably n is 1.

R₂ is selected from 4-fluorophenyl, 2-chloro-6-fluorophenyl, 2-trifluoromethylphenyl, 2-chloro-5-(5-fluoropyrimidin-2-yl)phenyl, 2-methylpyridin-3-yl, cyclohexyl, 4,4-difluorocyclohexyl, 3,3-difluorocyclopentyl, 6,6-difluorobicyclo[3.1.0]hexan-3-yl, 4-fluorocyclohexyl, 4-trifluoromethylcyclohexyl, cycloheptyl, 2-(trifluoromethyl)pyridin-4-yl, 4-spiro[2.5]octan-6-yl, 4,4-dimethylcyclohexyl, tetrahydrofuran-2-yl, 1-methylpiperidin-2-yl, morpholinyl, 2,2-dimethylcyclohexyl, 3,3-dimethylmorpholinyl, cycloheptylmethyl and OH.

Most preferably, a compound of formula (I) according to this invention is selected from the group consisting of:

| Exam- | |
|---|---|
| ple | IUPAC Name |
| 1 | 5-(2-chloro-6-fluorobenzyl)-4-(4-fluorobenzyl)-2-methyl-2,4- |
| | dihydro-3H-1,2,4-triazol-3-one |
| 2 | 5-(2-chloro-6-fluorobenzyl)-4-(cyclohexylmethyl)-2-methyl-2,4- |
| | dihydro-3H-1,2,4-triazol-3-one |
| 3 | 5-(2-chloro-6-fluorobenzyl)-4-((4,4-difluorocyclohexyl)methyl)- |
| | 2-methyl-2,4-dihydro-3H-1,2,4-triazol-3-one |
| 4 | 5-(2-chloro-6-fluorobenzyl)-4-((3,3-difluorocyclopentyl)methyl)- |
| | 2-methyl-2,4-dihydro-3H-1,2,4-triazol-3-one |
| 5 | 5-(2-chloro-6-fluorobenzyl)-4-((6,6-difluorobicyclo[3.1.0]hexan- |
| | 3-yl)methyl)-2-methyl-2,4-dihydro-3H-1,2,4-triazol-3-one |
| 6 | 5-(2-chloro-6-fluorobenzyl)-4-((4-fluorocyclohexyl)methyl)-2- |
| | methyl-2,4-dihydro-3H-1,2,4-triazol-3-one |
| 7 | 5-(2-chloro-6-fluorobenzyl)-2-methyl-4-((4- |
| | (trifluoromethyl)cyclohexyl)methyl)-2,4-dihydro-3H-1,2,4- |
| | triazol-3-one |
| 8 | 5-(2-chloro-4-fluorobenzyl)-4-(4-fluorobenzyl)-2-methyl-2,4- |
| | dihydro-3H-1,2,4-triazol-3-one |
| 9 | 5-(2-chloro-4-fluorobenzyl)-4-(cyclohexylmethyl)-2-methyl-2,4- |
| | dihydro-3H-1,2,4-triazol-3-one |
| 10 | 5-(2-chloro-4-fluorobenzyl)-4-((3,3-difluorocyclopentyl)methyl)- |
| | 2-methyl-2,4-dihydro-3H-1,2,4-triazol-3-one |
| 11 | 5-(2-chloro-4-fluorobenzyl)-4-(cycloheptylmethyl)-2-methyl-2,4- |
| | dihydro-3H-1,2,4-triazol-3-one |
| 12 | 5-(2-chloro-6-fluorobenzyl)-4-(cycloheptylmethyl)-2-methyl-2,4- |
| | dihydro-3H-1,2,4-triazol-3-one |
| 13 | 5-(cyclohexylmethyl)-2-methyl-4-((2-(trifluoromethyl)pyridin-4- |
| | yl)methyl)-2,4-dihydro-3H-1,2,4-triazol-3-one |
| 14 | 5-(2-chloro-6-fluorobenzyl)-2-methyl-4-(spiro[2.5]octan-6- |
| | ylmethyl)-2,4-dihydro-3H-1,2,4-triazol-3-one |
| 15 | 5-(2-chloro-6-fluorobenzyl)-4-(2-(4,4-difluorocyclohexyl)ethyl)- |
| | 2-methyl-2,4-dihydro-3H-1,2,4-triazol-3-one |
| 16 | 5-(2-chloro-6-fluorobenzyl)-4-(2-(4,4-dimethylcyclohexyl)ethyl)- |
| | 2-methyl-2,4-dihydro-3H-1,2,4-triazol-3-one |
| 17 | 4-(cyclohexylmethyl)-2-methyl-5-(2-(trifluoromethyl)benzyl)- |
| | 2,4-dihydro-3H-1,2,4-triazol-3-one |
| 18 | 4-((4,4-difluorocyclohexyl)methyl)-2-methyl-5-(2- |
| | (trifluoromethyl)benzyl)-2,4-dihydro-3H-1,2,4-triazol-3-one |
| 19 | 4-((3,3-difluorocyclopentyl)methyl)-2-methyl-5-(2- |
| | (trifluoromethyl)benzyl)-2,4-dihydro-3H-1,2,4-triazol-3-one |
| 20 | 5-(2-chloro-6-fluorobenzyl)-2-methyl-4-((tetrahydrofuran-2- |
| | yl)methyl)-2,4-dihydro-3H-1,2,4-triazol-3-one |
| 21 | 5-(2-chloro-6-fluorobenzyl)-2-methyl-4-((1-methylpiperidin-2- |
| | yl)methyl)-2,4-dihydro-3H-1,2,4-triazol-3-one |
| 22 | 5-(2-chloro-6-fluorobenzyl)-2-methyl-4-(2-morpholinoethyl)-2,4- |
| | dihydro-3H-1,2,4-triazol-3-one |
| 23 | 4-((2,2-dimethylcyclohexyl)methyl)-2-methyl-5-(2- |
| | (trifluoromethyl)benzyl)-2,4-dihydro-3H-1,2,4-triazol-3-one |
| 24 | 4-(2-chloro-6-fluorobenzyl)-5-(cyclohexylmethyl)-2-methyl-2,4- |
| | dihydro-3H-1,2,4-triazol-3-one |
| 25 | 5-(cyclohexylmethyl)-2-methyl-4-(2-(trifluoromethyl)benzyl)- |
| | 2,4-dihydro-3H-1,2,4-triazol-3-one |
| 26 | 4-((4,4-difluorocyclohexyl)methyl)-5-(2-fluorobenzyl)-2-methyl- |
| | 2,4-dihydro-3H-1,2,4-triazol-3-one |
| 27 | 4-(cyclohexylmethyl)-5-(2-fluorobenzyl)-2-methyl-2,4-dihydro- |
| | 3H-1,2,4-triazol-3-one |
| 28 | 4-(cycloheptylmethyl)-2-methyl-5-(2-(trifluoromethyl)benzyl)- |
| | 2,4-dihydro-3H-1,2,4-triazol-3-one |
| 29 | 4-(2-chloro-5-(5-fluoropyrimidin-2-yl)benzyl)-5-(4- |
| | fluorobenzyl)-2-methyl-2,4-dihydro-3H-1,2,4-triazol-3-one |
| 30 | 5-(2-chloro-6-fluorobenzyl)-4-(2-(3,3- |
| | dimethylmorpholino)ethyl)-2-methyl-2,4-dihydro-3H-1,2,4- |
| | triazol-3-one |
| 31 | 5-(cyclohexylmethyl)-2-methyl-4-((2-methylpyridin-3- |
| | yl)methyl)-2,4-dihydro-3H-1,2,4-triazol-3-one |
| 32 | 4-((2,2-dimethylcyclohexyl)methyl)-5-(2-fluorobenzyl)-2- |
| | methyl-2,4-dihydro-3H-1,2,4-triazol-3-one |
| 33 | 5-(2-chloro-6-fluorobenzyl)-4-((2,2-dimethylcyclohexyl)methyl)- |
| | 2-methyl-2,4-dihydro-3H-1,2,4-triazol-3-one |
| 34 | 5-(2-chloro-6-fluorobenzyl)-4-((4,4-difluorocyclohexyl)methyl)- |
| | 2-ethyl-2,4-dihydro-3H-1,2,4-triazol-3-one |
| 35 | 5-(2-chloro-4-fluorobenzyl)-2-methyl-4-(spiro[2.5]octan-6- |
| | ylmethyl)-2,4-dihydro-3H-1,2,4-triazol-3-one |
| 36 | 5-(2-chloro-4-fluorobenzyl)-4-(2-(4,4-difluorocyclohexyl)ethyl)- |
| | 2-methyl-2,4-dihydro-3H-1,2,4-triazol-3-one |
| 37 | 4-(cycloheptylmethyl)-5-(2-fluorobenzyl)-2-methyl-2,4-dihydro- |
| | 3H-1,2,4-triazol-3-one |
| 38 | 4-(2-chloro-5-(5-fluoropyrimidin-2-yl)benzyl)-5- |
| | (cyclohexylmethyl)-2-methyl-2,4-dihydro-3H-1,2,4-triazol-3-one |
| 39 | 4-(2-(4,4-difluorocyclohexyl)ethyl)-2-methyl-5-(2- |
| | (trifluoromethyl)benzyl)-2,4-dihydro-3H-1,2,4-triazol-3-one |
| 40 | 2-methyl-5-(2-(trifluoromethyl)benzyl)-4-((4- |
| | (trifluoromethyl)cyclohexyl)methyl)-2,4-dihydro-3H-1,2,4- |
| | triazol-3-one |
| 41 | 5-(2-chloro-4-fluorobenzyl)-4-(2-(4,4-dimethylcyclohexyl)ethyl)- |
| | 2-methyl-2,4-dihydro-3H-1,2,4-triazol-3-one |
| 42 | 5-(2-chlorobenzyl)-4-((4,4-difluorocyclohexyl)methyl)-2-methyl- |
| | 2,4-dihydro-3H-1,2,4-triazol-3-one |
| 43 | 5-(2-chloro-6-fluorobenzyl)-2-(cyclopropylmethyl)-4-((4,4- |
| | difluorocyclohexyl)methyl)-2,4-dihydro-3H-1,2,4-triazol-3-one |
| 44 | 5-(2-chloro-6-fluorobenzyl)-4-((4,4-difluorocyclohexyl)methyl)- |
| | 2-(2,2,2-trifluoroethyl)-2,4-dihydro-3H-1,2,4-triazol-3-one |
| 45 | 4-(2-(4,4-dimethylcyclohexyl)ethyl)-5-(2-fluorobenzyl)-2- |
| | methyl-2,4-dihydro-3H-1,2,4-triazol-3-one |
| 46 | 4-(2-(4,4-difluorocyclohexyl)ethyl)-5-(2-fluorobenzyl)-2-methyl- |
| | 2,4-dihydro-3H-1,2,4-triazol-3-one |
| 47 | 5-(2-fluorobenzyl)-2-methyl-4-(spiro[2.5]octan-6-ylmethyl)-2,4- |
| | dihydro-3H-1,2,4-triazol-3-one |
| 48 | 2-methyl-4-(spiro[2.5]octan-6-ylmethyl)-5-(2- |
| | (trifluoromethyl)benzyl)-2,4-dihydro-3H-1,2,4-triazol-3-one |
| 49 | 4-(2-(4,4-dimethylcyclohexyl)ethyl)-2-methyl-5-(2- |
| | (trifluoromethyl)benzyl)-2,4-dihydro-3H-1,2,4-triazol-3-one |
| 50 | 2-benzyl-5-(2-chloro-6-fluorobenzyl)-4-((4,4- |
| | difluorocyclohexyl)methyl)-2,4-dihydro-3H-1,2,4-triazol-3-one |
| 51 | 5-(2-fluorobenzyl)-2-methyl-4-((4- |
| | (trifluoromethyl)cyclohexyl)methyl)-2,4-dihydro-3H-1,2,4- |
| | triazol-3-one |
| 52 | 5-(2-chloro-4-fluorobenzyl)-2-methyl-4-((4- |
| | (trifluoromethyl)cyclohexyl)methyl)-2,4-dihydro-3H-1,2,4- |
| | triazol-3-one |
| 53 | 5-(2-chloro-6-fluorobenzyl)-4-(2-hydroxyethyl)-2-methyl-2,4- |
| | dihydro-3H-1,2,4-triazol-3-one |
| 54 | 5-[(2-chlorophenyl)methyl]-4-(cycloheptylmethyl)-2-(2,2,2- |
| | trifluoroethyl)-2,4-dihydro-3H-1,2,4-triazol-3-one |
| 55 | 5-[(2-chlorophenyl)methyl]-4-(cycloheptylmethyl)-2-methyl-2,4- |
| | dihydro-3H-1,2,4-triazol-3-one |
| 56 | 5-[(2-chloro-6-fluorophenyl)methyl]-2-methyl-4-[(1- |
| | methylcycloheptyl)methyl]-2,4-dihydro-3H-1,2,4-triazol-3-one |
| 57 | 5-[(2-chloro-6-fluorophenyl)methyl]-4-[(4,4- |
| | difluorocyclohexyl)methyl]-2-(3,3,3-trifluoropropyl)-2,4- |
| | dihydro-3H-1,2,4-triazol-3-one |
| 58 | 5-[(2-chloro-6-fluorophenyl)methyl]-4-[(4,4- |
| | difluorocyclohexyl)methyl]-2-(2,2-difluoroethyl)-2,4-dihydro- |
| | 3H-1,2,4-triazol-3-one |
| 59 | 4-[(2-chloro-6-fluorophenyl)methyl]-5-(cycloheptylmethyl)-2- |
| | methyl-2,4-dihydro-3H-1,2,4-triazol-3-one |
| 60 | 4-[(2-chloro-6-fluorophenyl)methyl]-5-(cycloheptylmethyl)-2- |
| | ethyl-2,4-dihydro-3H-1,2,4-triazol-3-one |
| 61 | 4-[(2-chloro-6-fluorophenyl)methyl]-5-(cycloheptylmethyl)-2- |
| | (2,2,2-trifluoroethyl)-2,4-dihydro-3H-1,2,4-triazol-3-one |
| 62 | 5-[(2-chlorophenyl)methyl]-4-[(4,4-difluorocyclohexyl)methyl]- |
| | 2-(2,2,2-trifluoroethyl)-2,4-dihydro-3H-1,2,4-triazol-3-one |
| 63 | 5-[(2-chlorophenyl)methyl]-4-[(4,4-difluorocyclohexyl)methyl]- |
| | 2-ethyl-2,4-dihydro-3H-1,2,4-triazol-3-one |
| 64 | 5-[(2-chlorophenyl)methyl]-4-(cycloheptylmethyl)-2-ethyl-2,4- |
| | dihydro-3H-1,2,4-triazol-3-one |
| 65 | 5-[(2-chloro-6-fluorophenyl)methyl]-4-[(3,3- |
| | difluorocyclopentyl)methyl]-2-ethyl-2,4-dihydro-3H-1,2,4- |
| | triazol-3-one |
| 66 | 5-[(2-chloro-6-fluorophenyl)methyl]-2-(cyclopropylmethyl)-4- |
| | [(3,3-difluorocyclopentyl)methyl]-2,4-dihydro-3H-1,2,4-triazol- |
| | 3-one |
| 67 | 5-[(2-chloro-6-fluorophenyl)methyl]-4-(cycloheptylmethyl)-2- |
| | (2,2,2-trifluoroethyl)-2,4-dihydro-3H-1,2,4-triazol-3-one |
| 68 | 5-[(2-chloro-6-fluorophenyl)methyl]-4-[(1-methylpiperidin-2- |
| | yl)methyl]-2-(2,2,2-trifluoroethyl)-2,4-dihydro-3H-1,2,4-triazol- |
| | 3-one |
| 69 | 5-(2-chloro-6-fluorobenzyl)-2-(cyclopropylmethyl)-4-((4- |
| | (trifluoromethyl)cyclohexyl)methyl)-2,4-dihydro-3H-1,2,4- |
| | triazol-3-one |
| 70 | 4-[(2-chloro-6-fluorophenyl)methyl]-5-[(4,4- |
| | difluorocyclohexyl)methyl]-2-(2,2,2-trifluoroethyl)-2,4-dihydro- |
| | 3H-1,2,4-triazol-3-one |
| 71 | 4-[(2-chloro-6-fluorophenyl)methyl]-5-[(4,4- |
| | difluorocyclohexyl)methyl]-2-methyl-2,4-dihydro-3H-1,2,4- |
| | triazol-3-one |

The compounds of formula (I) can generally be prepared by reacting a compound of formula (II):

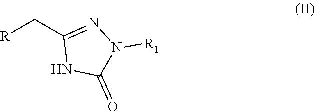

wherein R and R₁ are as defined above, with a compound of formula (III):

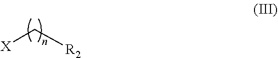

wherein R₂ and n are as defined above, and X is a suitable leaving group; and optionally converting the obtained compound of formula (I) into an addition salt thereof, and/or preparing stereochemically isomeric forms thereof.

X in the compound of Formula (III) is an appropriate leaving group such as, for example, halo, e.g. chloro, bromo, or in some instances X may also be an alcohol and the like reactive leaving groups. The reaction of a compound of formula (II) with a compound of formula (III), may be performed in a reaction-inert solvent such as, for example, acetonitrile or DMF, and optionally in the presence of a suitable base such as, for example, potassium carbonate or sodium methoxide. Stirring may enhance the rate of the reaction.

The reaction may conveniently be carried out at a temperature ranging between room temperature and the reflux temperature of the reaction mixture.

Compounds of formula (III) are known in the art.

Compounds of formula (II) can be prepared according to the following scheme:

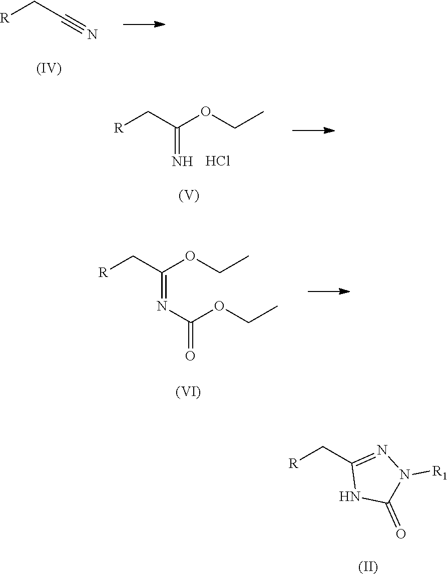

Compounds of formula (II), wherein R and R₁ are as defined in formula (I), can be obtained by cyclization of the respective ethyl-N-(ethoxycarbonyl)imidate derivatives (VI).

The reaction is performed in a suitable solvent, such as 1,4-dioxane, in the presence of hydrazine derivatives and TEA as base, preferably at a reflux temperature.

Compounds of formula (VI) wherein R is as defined in formula (I), can be prepared from the compounds of formula (V) by reaction with ethyl chloroformate in the presence of a tertiary amine, for example diisopropylethylamine, in an inert solvent, for example dichlormethane, preferably at 0-20° C.

Compounds of formula (V), wherein R is as defined in formula (I), can be prepared from opportune nitrile derivative (IV) by reaction with acetyl chloride, using EtOH such as solvent, preferably at room temperature.

The nitrile derivative (IV), are known compounds and are commercially available or may be prepared according to conventional reaction procedures generally known in the art. The compounds of formula (I), the pharmaceutically acceptable salts and stereoisomeric forms thereof possess P2X7 receptor antagonizing properties as demonstrated in the Pharmacological Examples. Other examples of art-known group transformation reactions to convert compounds of formula (I) into other compounds of formula (I) are hydrolysis of carboxylic esters to the corresponding carboxylic acid or alcohol; hydrolysis of amides to the corresponding carboxylic acids or amines; alcohols may be converted into esters and ethers; primary amines may be converted into secondary or tertiary amines; double bonds may be hydrogenated to the corresponding single bond. The starting materials and some of the intermediates are known compounds and are commercially available or may be prepared according to conventional reaction procedures generally known in the art. The compounds of formula (I) as prepared in the hereinabove described processes may be synthesized in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated there from by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials. In the preparation of the compounds of formula I and the starting materials and/or intermediates described herein it may be useful to protect certain groups which are sensitive to the reaction conditions. The evaluation of the usefulness of the optional protection, as well as the selection of the suitable protecting agent, according to the reaction carried out in the preparation of the compounds of the invention and the functional group to be protected, are within the common knowledge of the skilled person. The removal of the optional protective groups is carried out according to conventional techniques. For a general reference to the use of protective groups in organic chemistry, see Theodora W. Greene and Peter G. M. Wuts “Protective groups in organic synthesis”, John Wiley & Sons, Inc., II Ed., 1991.

The preparation of the salts of the compounds of formula I is carried out according to known methods. Therefore the present compounds of formula (I) are useful as a medicine especially in the treatment of a condition or disease mediated by the P2X7 receptor, in particular P2X7 receptor antagonistic activity. Subsequently the present compounds may be used for the manufacture of a medicine for treatment of a condition or a disease mediated by P2X7 receptor activity, in particular P2X7 receptor antagonistic activity.

The present invention also provides the use of a compound of formula (I) or a pharmaceutically acceptable salt thereof for the manufacture of a medicament for the treatment of conditions or diseases selected from P2X7 receptor mediated conditions or diseases. In an embodiment, the present invention provides a compound of formula (I) for use as a medicine or for use in the treatment of conditions or diseases selected from P2X7 receptor mediated conditions or diseases. Further, the present invention also provides a method of treatment of a condition mediated by P2X7 receptor activity, in a mammalian subject, which method comprises administering to a mammal in need of such treatment a therapeutically effective amount of a compound of formula (I) or a pharmaceutically acceptable salt thereof. In view of the above described mechanisms of action, the compounds of the invention are useful for the treatment of neurodegenerative disorders of various origins such as Alzheimer's Disease and other dementia conditions such as Lewys body, fronto-temporal dementia and taupathies; amyotrophic lateral sclerosis, Multiple Sclerosis, Parkinson's Disease and other parkinsonian syndromes; HIV-induced neuroinflammation; essential tremors; other spino cerebellar degenerations and Charcot-Marie-Toot neuropathy. The compounds of the invention are also useful for the treatment of neurological conditions such as epilepsy including simple partial seizure, complex partial seizure, secondary generalized seizure, further including absence seizure, myoclonic seizure, clonic seizure, tonic seizure, tonic clonic seizure and atonic seizure.

The compounds of the invention are also useful for the treatment of cognitive disorders and of psychiatric disorders. Psychiatric disorders include, and are not limited to major depression, dysthymia, mania, bipolar disorder (such as bipolar disorder type I, bipolar disorder type II), cyclothymic disorder, rapid cycling, ultradian cycling, mania, hypomania, schizophrenia, schizophreniform disorders, schizoaffective disorders, personality disorders, attention disorders with or without hyperactive behaviour, delusional disorders, brief psychotic disorders, shared psychotic disorders, psychotic disorder due to a general medical condition, substance-induced psychotic disorders or a psychotic disorder not otherwise specified, anxiety disorders such as generalised anxiety disorder, panic disorders, post-traumatic stress disorder, impulse control disorders, phobic disorders, dissociative states and moreover in smoke, drug addiction and alcoholism. In particular bipolar disorders, psychosis, anxiety and addiction.

The compounds of the present invention are useful in the prevention or treatment of neuropathic pain. Neuropathic pain syndromes include, and are not limited to: diabetic neuropathy; sciatica; non-specific lower back pain; multiple sclerosis pain; fibromyalgia; HIV-related neuropathy; neuralgia, such as post-herpetic neuralgia and trigeminal neuralgia, Morton's neuralgia, causalgia; and pain resulting from physical trauma, amputation, phantom limb, cancer, toxins or chronic inflammatory conditions; central pain such as the one observed in thalamic syndromes, mixed central and peripheral forms of pain such as complex regional pain syndromes (CRPS) also called reflex sympathetic dystrophies.

The compounds of the invention are also useful for the treatment of chronic pain. Chronic pain includes, and is not limited to, chronic pain caused by inflammation or an inflammatory-related condition, ostheoarthritis, rheumatoid arthritis, acute injury or trauma, upper back pain or lower back pain (resulting from systematic, regional or primary spine disease such as radiculopathy), bone pain (due to osteoarthritis, osteoporosis, bone metastasis or unknown reasons), pelvic pain, spinal cord injury-associated pain, cardiac chest pain, non-cardiac chest pain, central post-stroke pain, myofascial pain, sickle cell pain, cancer pain, Fabry's disease, AIDS pain, geriatric pain or pain caused by headache, temporomandibular joint syndrome, gout, fibrosis or thoracic outlet syndromes, in particular rheumatoid arthritis and osteoarthritis.

The compounds of the invention are also useful in the treatment of acute pain caused by acute injury, illness, sport-medicine injuries, carpal tunnel syndrome, burns, musculoskeletal sprains and strains, musculotendinous strain, cervicobrachial pain syndromes, dyspepsia, gastric ulcer, duodenal ulcer, dysmenorrhea, endometriosis or surgery (such as open heart or bypass surgery), post-operative pain, kidney stone pain, gallbladder pain, gallstone pain, obstetric pain or dental pain.

The compounds of the invention are also useful in the treatment of headaches such as migraine, tension type headache, transformed migraine or evolutive headache, cluster headache, as well as secondary headache disorders, such as the ones derived from infections, metabolic disorders or other systemic illnesses and other acute headaches, paroxysmal hemicrania and the like, resulting from a worsening of the above mentioned primary and secondary headaches.

Compounds of the invention are also useful in the treatment of diseases such as vertigo, tinnitus, muscle spasm, and other disorders including and not limited to cardiovascular diseases (such as cardiac arrhythmia, cardiac infarction or angina pectoris, hypertension, cardiac ischemia, cerebral ischemia) endocrine disorders (such as acromegaly or diabetes insipidus) diseases in which the pathophysiology of the disorder involves excessive or hypersecretory or otherwise inappropriate cellular secretion of an endogenous substance (such as catecholamine, a hormone or a growth factor).

The compounds of the invention are also useful in the selective treatment of liver disease, such as inflammatory liver diseases, for example chronic viral hepatitis B, chronic viral hepatitis C, alcoholic liver injury, primary biliary cirrhosis, autoimmune hepatitis, liver fibrosis, non-alcoholic steatohepatitis and liver transplant rejection.

The compounds of the invention inhibit inflammatory processes affecting all body systems. Therefore are useful in the treatment of inflammatory processes of the muscular-skeletal system of which the following is a list of examples but it is not comprehensive of all target disorders: arthritic conditions such as ankylosing spondylitis, cervical arthritis, fibromyalgia, gout, juvenile rheumatoid arthritis, lumbosacral arthritis, osteoarthritis, osteoporosis, psoriatic arthritis, rheumatic disease; disorders affecting skin and related tissues: eczema, psoriasis, dermatitis and inflammatory conditions such as sunburn; disorders of the respiratory system: asthma, allergic rhinitis and respiratory distress syndrome, lung disorders in which inflammation is involved such as asthma and bronchitis; chronic obstructive pulmonary disease; disorders of the immune and endocrinological systems: periarthritis nodosa, thyroiditis, aplastic anaemia, scleroderma, myasthenia gravis, multiple sclerosis and other demyelinating disorders, encephalomyelitis, sarcoidosis, nephritic syndrome, Bechet's syndrome, polymyositis, gingivitis.

Compounds of the invention are also useful in the treatment of gastrointestinal (GI) tract disorders such as inflammatory bowel disorders including but not limited to ulcerative colitis, Crohn's disease, ileitis, proctitis, celiac disease, enteropathies, microscopic or collagenous colitis, eosinophilic gastroenteritis, or pouchitis resulting after proctocolectomy and post ileonatal anastomosis, and irritable bowel syndrome including any disorders associated with abdominal pain and/or abdominal discomfort such as pylorospasm, nervous indigestion, spastic colon, spastic colitis, spastic bowel, intestinal neurosis, functional colitis, mucous colitis, laxative colitis and functional dyspepsia; but also for treatment of atrophic gastritis, gastritis varialoforme, ulcerative colitis, peptic ulceration, pyrosis, and other damage to the GI tract, for example, by Helicobacter pylori, gastroesophageal reflux disease, gastroparesis, such as diabetic gastroparesis; and other functional bowel disorders, such as non-ulcerative dyspepsia (NUD); emesis, diarrhoea, and visceral inflammation.

Compounds of the invention are also useful in the treatment of disorders of the genito-urinary tract such as overactive bladder, prostatitis (chronic bacterial and chronic non-bacterial prostatitis), prostadynia, interstitial cystitis, urinary incontinence and benign prostatic hyperplasia, annexities, pelvic inflammation, bartholinities and vaginitis. In particular, overactive bladder and urinary incontinence.

The compounds of the invention are also useful in the treatment of ophthalmic diseases such as retinitis, retinopathies, uveitis and acute injury to the eye tissue, age-related macular degeneration, chronic ocular hypertension, glaucoma, conjunctivitis.

The compounds of the invention are also useful in the treatment of eating disorders such as anorexia nervosa including the subtypes restricting type and binge-eating/purging type; bulimia nervosa including the subtypes purging type and non-purging type; obesity; compulsive eating disorders; binge eating disorder; and eating disorder not otherwise specified.

The compounds of the invention are also useful in the treatment of allergic dermatitis, hyper-responsiveness of the airway, chronic obstructive pulmonary disease (COPD), bronchitis, septic shock, Sjögren's syndrome, glomerulonephritis, atherosclerosis, growth and metastases of malignant cells, myoblastic leukaemia, diabetes, meningitis, osteoporosis, burn injury, ischaemic heart disease, stroke, peripheral vascular disease, varicose veins, glaucoma.

The term “treating” and “treatment’, as used herein, refers to curative, palliative and prophylactic treatment, including reversing, alleviating, inhibiting the progress of, or preventing the disease, disorder or condition to which such term applies, or one or more symptoms of such disease, disorder or condition.

Additionally the present invention provides pharmaceutical compositions comprising at least one pharmaceutically acceptable carrier and a therapeutically effective amount of a compound of formula (I).

In order to prepare the pharmaceutical compositions of this invention, an effective amount of the particular compound, in base or acid addition salt form, as the active ingredient is combined in intimate admixture with at least one pharmaceutically acceptable carrier, which carrier may take a wide variety of forms depending on the form of preparation desired for administration. These pharmaceutical compositions are desirably in unitary dosage form suitable, preferably, for oral administration, rectal administration, percutaneous administration or parenteral injection.

For example in preparing the compositions in oral dosage form, any of the usual liquid pharmaceutical carriers may be employed, such as for instance water, glycols, oils, alcohols and the like in the case of oral liquid preparations such as suspensions, syrups, elixirs and solutions; or solid pharmaceutical carriers such as starches, sugars, kaolin, lubricants, binders, disintegrating agents and the like in the case of powders, pills, capsules and tablets. Because of their easy administration, tablets and capsules represent the most advantageous oral dosage unit form, in which case solid pharmaceutical carriers are obviously employed. For parenteral injection compositions, the pharmaceutical carrier will mainly comprise sterile water, although other ingredients may be included in order to improve solubility of the active ingredient.

Injectable solutions may be prepared for instance by using a pharmaceutical carrier comprising a saline solution, a glucose solution or a mixture of both. Injectable suspensions may also be prepared by using appropriate liquid carriers, suspending agents and the like. In compositions suitable for percutaneous administration, the pharmaceutical carrier may optionally comprise a penetration enhancing agent and/or a suitable wetting agent, optionally combined with minor proportions of suitable additives which do not cause a significant deleterious effect to the skin. Said additives may be selected in order to facilitate administration of the active ingredient to the skin and/or be helpful for preparing the desired compositions. These topical compositions may be administered in various ways, e.g., as a transdermal patch, a spot-on or an ointment. Addition salts of the compounds of formula (1), due to their increased water solubility over the corresponding base form, are obviously more suitable in the preparation of aqueous compositions.

It is especially advantageous to formulate the pharmaceutical compositions of the invention in dosage unit form for ease of administration and uniformity of dosage.

“Dosage unit form” as used herein refers to physically discrete units suitable as unitary dosages, each unit containing a predetermined amount of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

For oral administration, the pharmaceutical compositions of the present invention may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, tale, silica and the like), disintegrating agents (e.g. potato starch, sodium starch glycollate and the like), wetting agents (e.g. sodium lauryl sulphate) and the like. Such tablets may also be coated by methods well known in the art.

Liquid preparations for oral administration may take the form of e.g. solutions, syrups or suspensions, or they may be formulated as a dry product for admixture with water and/or another suitable liquid carrier before use. Such liquid preparations may be prepared by conventional means, optionally with other pharmaceutically acceptable additives such as suspending agents (e.g. sorbitol syrup, methylcellulose, hydroxypropylmethylcellulose or hydrogenated edible fats), emulsifying agents (e.g. lecithin or acacia), non-aqueous carriers (e.g. almond oil, oily esters or ethyl alcohol), sweeteners, flavours, masking agents and preservatives (e.g. methyl or propyl p-hydroxybenzoates or sorbic acid).

Pharmaceutically acceptable sweeteners useful in the pharmaceutical compositions of the invention comprise preferably at least one intense sweetener such as aspartame, acesulfame potassium, sodium cyclamate, alitarne, a dihydrochalcone sweetener, monellin, stevioside sucralose (4,1′, 6′-trichloro-4, 1′,6′-trideoxygalactosucrose) or, preferably, saccharin, sodium or calcium saccharin, and optionally at least one bulk sweetener such as sorbitol, mannitol, fructose, sucrose, maltose, isomalt, glucose, hydrogenated glucose syrup, xylitol, caramel or honey. Intense sweeteners are conveniently used in low concentrations. For example, in the case of sodium saccharin, the said concentration may range from about 0.04% to 0.1% (weight/volume) of the final formulation. The bulk sweetener can effectively be used in larger concentrations ranging from about 10% to about 35%, preferably from about 10% to 15% (weight/volume). The pharmaceutically acceptable flavours which can mask the bitter tasting ingredients in the low-dosage formulations comprise preferably fruit flavours such as cherry, raspberry, black currant or strawberry flavour. A combination of two flavours may yield very good results. In the high-dosage formulations, stronger pharmaceutically acceptable flavours may be required such as Caramel Chocolate, Mint Cool, Fantasy and the like.

Each flavour may be present in the final composition in a concentration ranging from about 0.05% to 1% (weight/volume). Combinations of said strong flavours are advantageously used. Preferably a flavour is used that does not undergo any change or loss of taste and/or color under the circumstances of the formulation.

The compounds of formula (I) may be formulated for parenteral administration by injection, conveniently intravenous, intra-muscular or subcutaneous injection, for example by bolus injection or continuous intravenous infusion. Formulations for injection may be presented in unit dosage form, e.g. in ampoules or multi-dose containers, including an added preservative. They may take such forms as suspensions, solutions or emulsions in oily or aqueous vehicles, and may contain formulating agents such as isotonizing, suspending, stabilizing and/or dispersing agents. Alternatively, the active ingredient may be present in powder form for mixing with a suitable vehicle, e.g. sterile pyrogen-free water, before use.

The compounds of formula (I) may also be formulated in rectal compositions such as suppositories or retention enemas, e.g. containing conventional suppository bases such as cocoa butter and/or other glycerides.

Those of skill in the treatment of diseases linked to the mediation of the ligand-gated ion channels will easily determine the therapeutically effective amount of a compound of formula (I) from the test results presented hereinafter. In general it is contemplated that a therapeutically effective dose will be from about 0.001 mg/kg to about 50 mg/kg of body weight, more preferably from about 0.01 mg/kg to about 10 mg/kg of body weight of the patient to be treated. It may be appropriate to administer the therapeutically effective dose in the form of two or more sub-doses at appropriate intervals throughout the day. Said sub-doses may be formulated as unit dosage forms, for example each containing from about 0.1 mg to about 1000 mg, more particularly from about 1 to about 500 mg, of the active ingredient per unit dosage form.

As used herein, a “therapeutically effective amount” of a compound, is the quantity of a compound which, when administered to an individual or animal, results in a sufficiently high level of that compound in the individual or animal to cause a discernible P2X7 receptor antagonistic response.

The exact dosage and frequency of administration depends on the particular compound of formula (I) used, the particular condition being treated, the severity of the condition being treated, the age, weight and general physical condition of the particular patient as well as the other medication, the patient may be taking, as is well known to those skilled in the art. Furthermore, said “therapeutically effective amount” may be lowered or increased depending on the response of the treated patient and/or depending on the evaluation of the physician prescribing the compounds of the instant invention. The effective daily amount ranges mentioned hereinabove are therefore only guidelines.

### Nomenclature and Structures

In general, the nomenclature used in this Application is based on ChemOffice and generated according to the IUPAC systematic nomenclature. Chemical structures shown herein were prepared using ChemDraw version 18.2. Any open valency appearing on a carbon, oxygen, sulfur, or nitrogen atom in the structures herein indicates the presence of a hydrogen atom unless indicated otherwise. Where a nitrogen-containing heteroaryl ring is shown with an open valency on a nitrogen atom and variables such as R¹, R², R³ etc. are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structure herein are intended to encompass all naturally occurring isotopes of such atoms. Thus, for example, the hydrogen atoms represented herein are meant to include deuterium and tritium, and the carbon atoms are meant to include ¹³C and ¹⁴C isotopes.

### Abbreviations

Abbreviations which may be used in the description of the Schemes and the Examples that follows are:
CC: Column Chromatography;
DCM: Dichloromethane; DMF: Dimethylformamide;
EtOAc: Ethyl acetate;
EtOH: Ethanol;
hrs: hours;
ACN: Acetonitrile;
min: Minute(s); N: Normal;
NMR: Nuclear Magnetic Resonance;
r.t.: Room Temperature;
THF: Tetrahydrofuran; LC-MS: Liquid Chromatography-Mass Spectrometry;
K₂CO₃: potassium carbonate;
Na₂SO₄: sodium sulphate;
HPLC: High-performance liquid chromatography; o.n.: overnight;
CH₃ONa: sodium methoxide;
NaCl: sodium chloride;
HCl: Hydrochloric acid;
Y: yield;
DIPEA: N,N-Diisopropylethylamine;
Et₂O: Diethylether;
LiAlH₄: Lithium aluminum hydride.

### Experimental Part

The following examples illustrate the present invention. Unless explicitly stated otherwise, all particulars (especially percentages and amounts) relate to the weight.

### Intermediates

### A. Nitrile Derivatives

Most substituted nitrile derivatives used, as starting materials, were purchased from chemical providers:

| Structure of nitrile derivatives | CAS number |
|---|---|
| 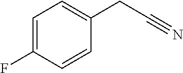 | 459-22-3 |
| 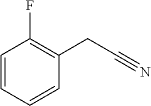 | 326-62-5 |
| 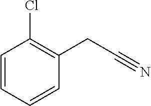 | 2856-63-5 |
| 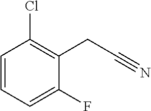 [SMILES: N#CCc1c(F)cccc1Cl] | 75279-55-9 |
| 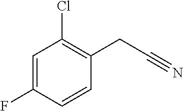 [SMILES: N#CCc1ccc(F)cc1Cl] | 75279-56-0 |
| 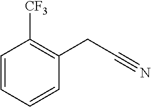 [SMILES: N#CCc1ccccc1C(F)(F)F] | 3038-47-9 |
| 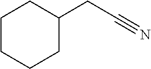 | 4435-14-7 |
| 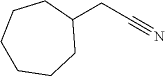 | 5452-65-3 |
| 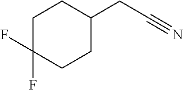 [SMILES: N#CCC1CCC(F)(F)CC1] | 959600-88-5 |

### B. Halogen Derivatives

Most halogen derivatives were purchased from chemical providers:

| Structure of halide derivatives | CAS number |
|---|---|
| 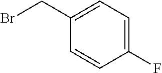 | 459-46-1 |
| 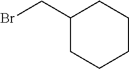 | 2550-36-9 |
| 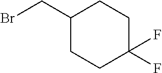 | 858121-94-5 |
| 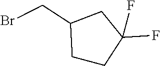 | 1695914-13-6 |
| 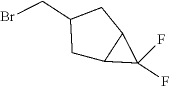 | 1393569-74-8 |
| 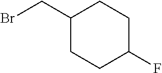 | 1784609-74-0 |
| 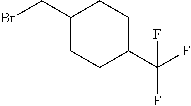 [SMILES: FC(F)(F)C1CCC(CBr)CC1] | 858121-96-7 |
| 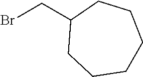 | 3814-32-2 |
| 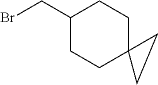 | 1621225-50-0 |
| 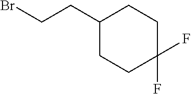 [SMILES: FC1(F)CCC(CCBr)CC1] | 2092565-10-9 |
| 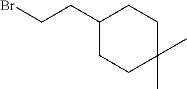 | 570398-26-4 |
| 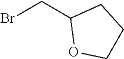 | 1192-30-9 |
| 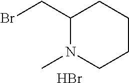 | 1390654-84-8 |
| 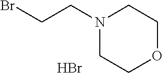 | 42802-94-8 |
| 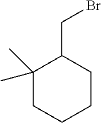 | 1501249-61-1 |
| 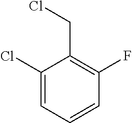 | 55117-15-2 |
| 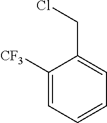 [SMILES: FC(F)(F)c1ccccc1CCl] | 21742-00-7 |
| 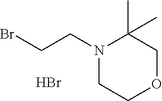 | 1098202-59-5 |
| 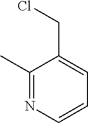 | 120277-68-1 |
| 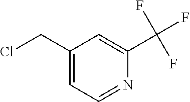 [SMILES: FC(F)(F)c1cc(CCl)ccn1] | 1027545-48-7 |
| 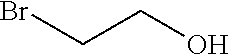 | 540-51-2 |

### General Synthetic Schemes

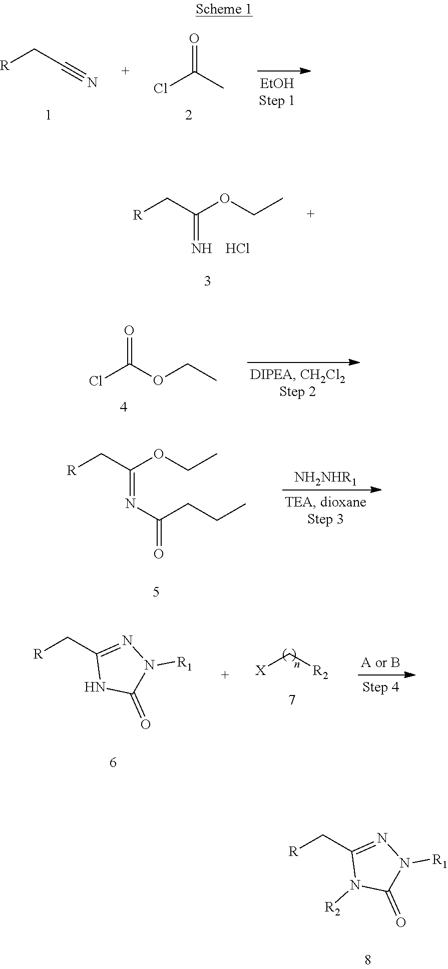

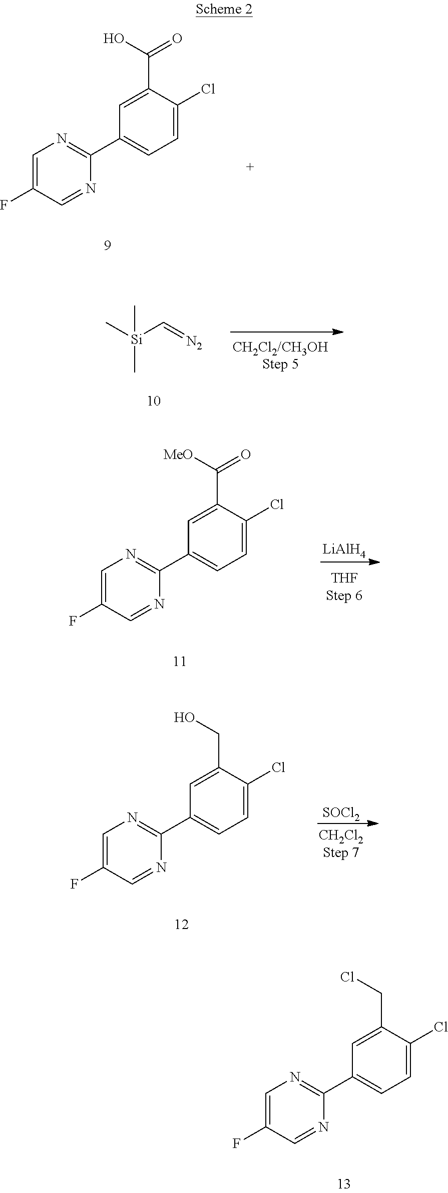

Step 1. Acetyl chloride 2 (8.0 eq) was added dropwise to a cooled (0° C.) and stirred solution of nitrile derivative 1 (1.0 eq) in EtOH; the reaction flask was stoppered tightly, and the reaction mixture was allowed to warm up to room temperature and stirred overnight. After the reaction completion by LC-MS analysis, the volatiles were removed under reduced pressure to isolate the product as hydrochloride. The residue was used without further purification.

Step 2. To a cooled (0° C.) and stirred solution of 3 (1.0 eq) in DCM, under a N₂ flow, DIPEA (3.0 eq) was added, and the reaction mixture was stirred at 0° C. for 30 minutes. Then, ethyl chloroformate (1.1 eq) was added dropwise into the reaction mixture within 30-45 minutes. The reaction mixture was stirred 3 h at room temperature. The mixture was then filtered through a silica plug in order to remove the salts and filtrate was concentrated under reduced pressure. The desired product was obtained after purification of the crude product as reported in the specific examples.

Step 3. To solution of 5 (1.0 eq) in dioxane hydrazine derivative (2.5 eq) and TEA (2.5 eq) were added and the reaction mixture was heated to reflux for 16 h. After being cooled, the mixture was diluted with water and washed with EtOAc. Then, the pH was adjusted to 2 with HCl 1M and the aqueous solution was extracted with EtOAc. The organic phase was then washed with brine, dried (Na₂SO₄) and evaporated under vacuum. The desired product was obtained after purification of the crude product as reported in the specific examples.

### Step 4.

Method A. To a cooled (0° C.) and stirred solution of 6 (1.0 eq) in DMF, CH₃ONa (3 eq) was added, and the reaction mixture was stirred at the same temperature for 10 minutes. Then, the halide 7 (5 eq) was added and the reaction mixture was stirred to 700 overnight. The reaction was quenched by addition of water and extracted with EtOAc (x 3). The organic layers were combined, washed with brine, dried (Na₂SO₄) and evaporated under reduced pressure. The desired product was obtained after purification of the crude product as reported in the specific examples.

Method B. To a cooled (0° C.) and stirred solution of 6 (1.0 eq) in ACN/DMF (5:1 v/v) K₂CO₃ (2.5 eq) was added, and the reaction mixture was stirred at the same temperature for 10 minutes. Then, the halide 7 (1.2 eq) was added and the reaction mixture was stirred to r.t. overnight. The reaction was quenched by addition of water and extracted with EtOAc (x 3). The organic layers were combined, washed with brine, dried (Na₂SO₄) and evaporated under reduced pressure. The desired product was obtained after purification of the crude product as reported in the specific examples.

Step 5. To a solution of benzoic acid derivative (9) (1.0 eq.) in MeOH and DCM (3.5 ml+1.5 ml) (trimethylsilyl)diazomethane (10) was added dropwise. The reaction was allowed to stir at room temperature for 2 hours. Then two more equivalents of (trimethylsilyl)diazomethane were added and the reaction was allowed to stir at room temperature for additional 2 hours. The solvent was then removed under reduced pressure and the remaining residue was taken up in EtOAc. The solution was washed with NaHCO₃ s.s. (2×10 mL), dried (Na₂SO₄) and filtered. The solvent was removed under reduced pressure and the residue was used for the next step without further purification.

Step 6. To a solution of 11 (1.0 eq) in THE at 0° C. LiAlH₄(0.5 eq) was added under vigorous stirring and the reaction was warmed up at rt. The mixture was then stirred at rt for 2 h. After completion, the mixture was then cooled to 0° C., quenched with H₂O and diluted with the addition of Et₂O. The reaction mixture was filtered under vacuum, the filtrate was dried (Na₂SO₄) and concentrated under vacuum. The desired product was obtained after purification of the crude product as reported in the specific examples.

Step 7. A mixture of 12 (1.0 eq) and thionyl chloride (1.5 eq) in DCM was heated at 45° C. overnight. After cooling to rt, solvent was removed under vacuo and the residue was used for the next step without further purification.

### SPECIFIC EXAMPLES

### Example 1

### 5-(2-chloro-6-fluorobenzyl)-4-(4-fluorobenzyl)-2-methyl-2,4-dihydro-3H-1,2,4-triazol-3-one

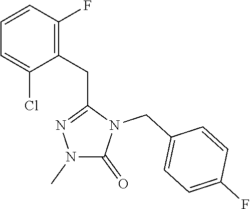 [SMILES: Cn1nc(Cc2c(F)cccc2Cl)n(Cc2ccc(F)cc2)c1=O]

ethyl 2-(2-chloro-6-fluorophenyl)acetimidate hydrochloride (intermediate 1). The title compound was prepared by the general procedure (scheme 1, step 1) starting from commercially available nitrile CAS: 75279-55-9.

ethyl-2-(2-chloro-6-fluorophenyl)-N-(ethoxycarbonyl)acetimidate (intermediate 2). The title compound was prepared by the general procedure (scheme 1, step 2) starting from intermediate 1. It was purified by flash silica gel column chromatography using Hexane/EtOAc 4:1 v/v as the eluent (Y=19%).

5-(2-chloro-6-fluorobenzyl)-2-methyl-2,4-dihydro-3H-1,2,4-triazol-3-one (intermediate 3). The title compound was prepared by the general procedure (scheme 1, step 3) starting from intermediate 2 and methyl hydrazine CAS: 60-34-4. It was purified by flash silica gel column chromatography using Hexane/AcOEt 1:4 v/v as the eluent (Y=59%).

5-(2-chloro-6-fluorobenzyl)-4-(4-fluorobenzyl)-2-methyl-2,4-dihydro-3H-1,2,4-triazol-3-one. The title compound was prepared by the general procedure (scheme 1, step 4, method A) starting from intermediate 3 and bromide CAS: 459-46-1. It was purified by HPLC (Y=64%).

### Example 2

### 5-(2-chloro-6-fluorobenzyl)-4-(cyclohexylmethyl)-2-methyl-2,4-dihydro-3H-1,2,4-triazol-3-one

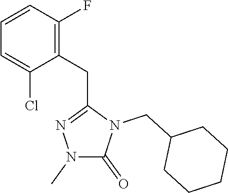 [SMILES: Cn1nc(Cc2c(F)cccc2Cl)n(CC2CCCCC2)c1=O]

The title compound was prepared by the general procedure (scheme 1, step 4, method A) starting from intermediate 3 and bromide CAS: 2550-36-9. It was purified by HPLC (Y=58%).

### Example 3

### 5-(2-chloro-6-fluorobenzyl)-4-((4,4-difluorocyclohexyl)methyl)-2-methyl-2,4-dihydro-3H-1,2,4-triazol-3-one

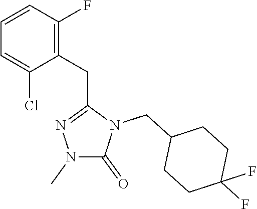 [SMILES: Cn1nc(Cc2c(F)cccc2Cl)n(CC2CCC(F)(F)CC2)c1=O]

The title compound was prepared by the general procedure (scheme 1, step 4, method A) starting from intermediate 3 and bromide CAS: 858121-94-5. It was purified by HPLC (Y=35%).

### Example 4

### 5-(2-chloro-6-fluorobenzyl)-4-((3,3-difluorocyclopentyl)methyl)-2-methyl-2,4-dihydro-3H-1,2,4-triazol-3-one

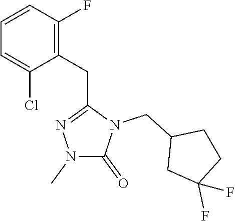 [SMILES: Cn1nc(Cc2c(F)cccc2Cl)n(CC2CCC(F)(F)C2)c1=O]

The title compound was prepared by the general procedure (scheme 1, step 4, method A) starting from intermediate 3 and bromide CAS: 1695914-13-6. It was purified by HPLC (Y=73%).

### Example 5

### 5-(2-chloro-6-fluorobenzyl)-4-((6,6-difluorobicyclo[3.1.0]hexan-3-yl)methyl)-2-methyl-2,4-dihydro-3H-1,2,4-triazol-3-one

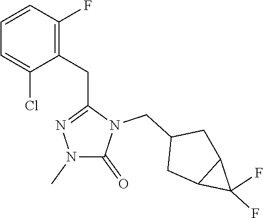 [SMILES: Cn1nc(Cc2c(F)cccc2Cl)n(CC2CC3C(C2)C3(F)F)c1=O]

The title compound was prepared by the general procedure (scheme 1, step 4, method A) starting from intermediate 3 and bromide CAS: 1393569-74-8. It was purified by HPLC (Y=65%).

### Example 6

### 5-(2-chloro-6-fluorobenzyl)-4-((4-fluorocyclohexyl)methyl)-2-methyl-2,4-dihydro-3H-1,2,4-triazol-3-one

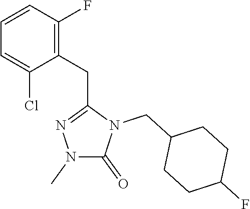 [SMILES: Cn1nc(Cc2c(F)cccc2Cl)n(CC2CCC(F)CC2)c1=O]

The title compound was prepared by the general procedure (scheme 1, step 4, method A) starting from intermediate 3 and bromide CAS: 1784609-74-0. It was purified by HPLC (Y=29%).

### Example 7

### 5-(2-chloro-6-fluorobenzyl)-2-methyl-4-((4-(trifluoromethyl)cyclohexyl)methyl)-2,4-dihydro-3H-1,2,4-triazol-3-one

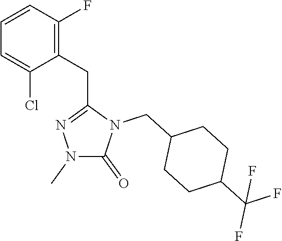 [SMILES: Cn1nc(Cc2c(F)cccc2Cl)n(CC2CCC(C(F)(F)F)CC2)c1=O]

The title compound was prepared by the general procedure (scheme 1, step 4, method A) starting from intermediate 3 and bromide CAS: 858121-96-7. It was purified by HPLC (Y=39%).

### Example 8

### 5-(2-chloro-4-fluorobenzyl)-4-(4-fluorobenzyl)-2-methyl-2,4-dihydro-3H-1,2,4-triazol-3-one

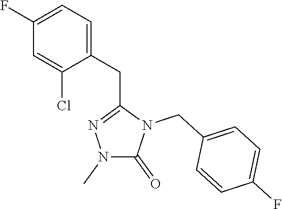 [SMILES: Cn1nc(Cc2ccc(F)cc2Cl)n(Cc2ccc(F)cc2)c1=O]

ethyl 2-(2-chloro-4-fluorophenyl)acetimidate hydrochloride (intermediate 4). The title compound was prepared by the general procedure (scheme 1, step 1) starting from commercially available nitrile CAS: 75279-56-0.

ethyl-2-(2-chloro-4-fluorophenyl)-N-(ethoxycarbonyl)acetimidate (intermediate 5). The title compound was prepared by the general procedure (scheme 1, step 2) starting from intermediate 4. It was purified by flash silica gel column chromatography using Hexane/EtOAc 4:1 v/v as the eluent (Y=64%).

5-(2-chloro-4-fluorobenzyl)-2-methyl-2,4-dihydro-3H-1,2,4-triazol-3-one (intermediate 6). The title compound was prepared by the general procedure (scheme 1, step 3) starting from intermediate 5 and methyl hydrazine CAS: 60-34-4. It was purified by flash silica gel column chromatography using Hexane/EtOAc 4:1 v/v as the eluent (Y=91%).

5-(2-chloro-4-fluorobenzyl)-4-(4-fluorobenzyl)-2-methyl-2,4-dihydro-3H-1,2,4-triazol-3-one. The title compound was prepared by the general procedure (scheme 1, step 4, method A) starting from intermediate 6 and bromide CAS: 459-46-1. It was purified by HPLC (Y=89%).

### Example 9

### 5-(2-chloro-4-fluorobenzyl)-4-(cyclohexylmethyl)-2-methyl-2,4-dihydro-3H-1,2,4-triazol-3-one

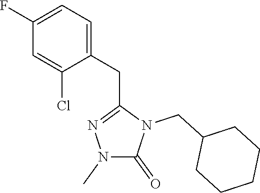 [SMILES: Cn1nc(Cc2ccc(F)cc2Cl)n(CC2CCCCC2)c1=O]

The title compound was prepared by the general procedure (scheme 1, step 4, method A) starting from intermediate 6 and bromide CAS: 2550-36-9. It was purified by HPLC (Y=66%).

### Example 10

### 5-(2-chloro-4-fluorobenzyl)-4-((3,3-difluorocyclopentyl)methyl)-2-methyl-2,4-dihydro-3H-1,2,4-triazol-3-one

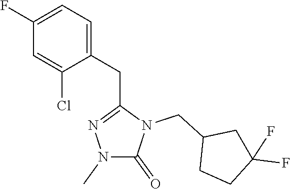 [SMILES: Cn1nc(Cc2ccc(F)cc2Cl)n(CC2CCC(F)(F)C2)c1=O]

The title compound was prepared by the general procedure (scheme 1, step 4, method A) starting from intermediate 6 and bromide CAS: 1695914-13-6. It was purified by HPLC (Y=58%).

### Example 11

### 5-(2-chloro-4-fluorobenzyl)-4-(cycloheptylmethyl)-2-methyl-2,4-dihydro-3H-1,2,4-triazol-3-one

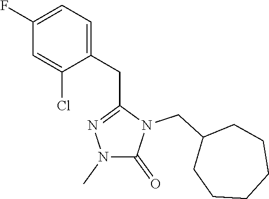 [SMILES: Cn1nc(Cc2ccc(F)cc2Cl)n(CC2CCCCCC2)c1=O]

The title compound was prepared by the general procedure (scheme 1, step 4, method A) starting from intermediate 6 and bromide CAS: 3814-32-2. It was purified by HPLC (Y=57%).

### Example 12

### 5-(2-chloro-6-fluorobenzyl)-4-(cycloheptylmethyl)-2-methyl-2,4-dihydro-3H-1,2,4-triazol-3-one

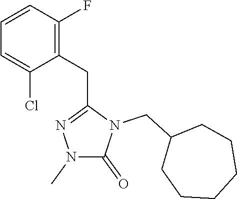 [SMILES: Cn1nc(Cc2c(F)cccc2Cl)n(CC2CCCCCC2)c1=O]

The title compound was prepared by the general procedure (scheme 1, step 4, method A) starting from intermediate 3 and bromide CAS: 3814-32-2. It was purified by HPLC (Y=44%).

### Example 13

### 5-(cyclohexylmethyl)-2-methyl-4-((2-(trifluoromethyl)pyridin-4-yl)methyl)-2,4-dihydro-3H-1,2,4-triazol-3-one

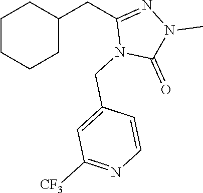 [SMILES: Cn1nc(CC2CCCCC2)n(Cc2ccnc(C(F)(F)F)c2)c1=O]

ethyl 2-cyclohexylacetimidate hydrochloride (intermediate 7). The title compound was prepared by the general procedure (scheme 1, step 1) starting from commercially available nitrile CAS: 4435-14-7.

ethyl-2-cyclohexyl-N-(ethoxycarbonyl)acetimidate (intermediate 8). The title compound was prepared by the general procedure (scheme 1, step 2) starting from intermediate 7. It was purified by flash silica gel column chromatography using a linear gradient of ethyl acetate in hexane from 0 to 50% (Y=49%).

5-(cyclohexylmethyl)-2-methyl-2,4-dihydro-3H-1,2,4-triazol-3-one (intermediate 9). The title compound was prepared by the general procedure (scheme 1, step 3) starting from intermediate 8 and methyl hydrazine CAS: 60-34-4. It was purified by flash silica gel column chromatography using a linear gradient of ethyl acetate in hexane from 0 to % 100% (Y=64%).

5-(cyclohexylmethyl)-2-methyl-4-((2-(trifluoromethyl)pyridin-4-yl)methyl)-2,4-dihydro-3H-1,2,4-triazol-3-one. The title compound was prepared by the general procedure (scheme 1, step 4, method B) starting from intermediate 9 and chloride CAS: 1027545-48-7. It was purified by HPLC (Y=37%).

### Example 14

### 5-(2-chloro-6-fluorobenzyl)-2-methyl-4-(spiro[2.5]octan-6-ylmethyl)-2,4-dihydro-3H-1,2,4-triazol-3-one

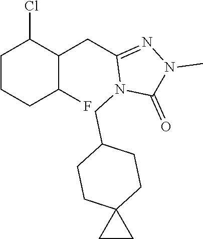 [SMILES: Cn1nc(CC2C(F)CCCC2Cl)n(CC2CCC3(CC2)CC3)c1=O]

The title compound was prepared by the general procedure (scheme 1, step 4, method A) starting from intermediate 3 and bromide CAS: 1621225-50-0. It was purified by HPLC (Y=53%).

### Example 15

### 5-(2-chloro-6-fluorobenzyl)-4-(2-(4,4-difluorocyclohexyl)ethyl)-2-methyl-2,4-dihydro-3H-1,2,4-triazol-3-one

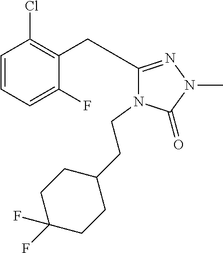 [SMILES: Cn1nc(Cc2c(F)cccc2Cl)n(CCC2CCC(F)(F)CC2)c1=O]

The title compound was prepared by the general procedure (scheme 1, step 4, method A) starting from intermediate 3 and bromide CAS: 2092565-10-9. It was purified by HPLC (Y=31%).

### Example 16

### 5-(2-chloro-6-fluorobenzyl)-4-(2-(4,4-dimethylcyclohexyl)ethyl)-2-methyl-2,4-dihydro-3H-1,2,4-triazol-3-one

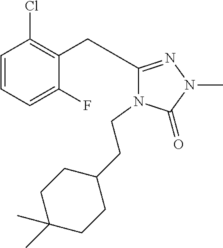 [SMILES: Cn1nc(Cc2c(F)cccc2Cl)n(CCC2CCC(C)(C)CC2)c1=O]

The title compound was prepared by the general procedure (scheme 1, step 4, method A) starting from intermediate 3 and bromide CAS: 570398-26-4. It was purified by HPLC (Y=57%).

### Example 17

### 4-(cyclohexylmethyl)-2-methyl-5-(2-(trifluoromethyl)benzyl)-2,4-dihydro-3H-1,2,4-triazol-3-one

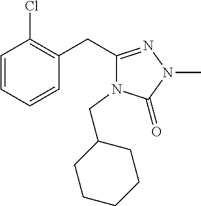 [SMILES: Cn1nc(Cc2ccccc2Cl)n(CC2CCCCC2)c1=O]

ethyl 2-(2-(trifluoromethyl)phenyl)acetimidate hydrochloride (intermediate 10). The title compound was prepared by the general procedure (scheme 1, step 1) starting from commercially available nitrile CAS: 3038-47-9.

ethyl-N-(ethoxycarbonyl)-2-(2-(trifluoromethyl)phenyl)acetimidate (intermediate 11). The title compound was prepared by the general procedure (scheme 1, step 2) starting from intermediate 10. It was purified by flash silica gel column chromatography using a linear gradient of ethyl acetate in cyclohexane from 2 to 15% (Y=35%).

2-methyl-5-(2-(trifluoromethyl)benzyl)-2,4-dihydro-3H-1,2,4-triazol-3-one (intermediate 12). The title compound was prepared by the general procedure (scheme 1, step 3) starting from intermediate 11 and methyl hydrazine CAS: 60-34-4. It was used for the next step without further purification (Y=97%).

4-(cyclohexylmethyl)-2-methyl-5-(2-(trifluoromethyl)benzyl)-2,4-dihydro-3H-1,2,4-triazol-3-one. The title compound was prepared by the general procedure (scheme 1, step 4, method A) starting from intermediate 12 and bromide CAS: 2550-36-9. It was purified by HPLC (Y=27%).

### Example 18

### 4-((4,4-difluorocyclohexyl)methyl)-2-methyl-5-(2-(trifluoromethyl)benzyl)-2,4-dihydro-3H-1,2,4-triazol-3-one

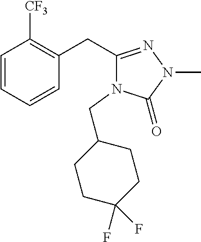 [SMILES: Cn1nc(Cc2ccccc2C(F)(F)F)n(CC2CCC(F)(F)CC2)c1=O]

The title compound was prepared by the general procedure (scheme 1, step 4, method A) starting from intermediate 12 and bromide CAS: 858121-94-5. It was purified by HPLC (Y=23%).

### Example 19

### 4-((3,3-difluorocyclopentyl)methyl)-2-methyl-5-(2-(trifluoromethyl)benzyl)-2,4-dihydro-3H-1,2,4-triazol-3-one

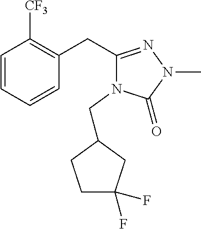 [SMILES: Cn1nc(Cc2ccccc2C(F)(F)F)n(CC2CCC(F)(F)C2)c1=O]

The title compound was prepared by the general procedure (scheme 1, step 4, method A) starting from intermediate 12 and bromide CAS: 1695914-13-6. It was purified by HPLC (Y=24%).

### Example 20

### 5-(2-chloro-6-fluorobenzyl)-2-methyl-4-((tetrahydrofuran-2-yl)methyl)-2,4-dihydro-3H-1,2,4-triazol-3-one

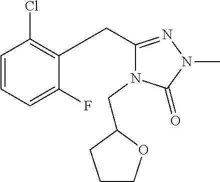 [SMILES: Cn1nc(Cc2c(F)cccc2Cl)n(CC2CCCO2)c1=O]

The title compound was prepared by the general procedure (scheme 1, step 4, method A) starting from intermediate 3 and bromide CAS: 1192-30-9. It was purified by flash silica gel column chromatography using a linear gradient of ethyl acetate in cyclohexane from 0 to 100% (Y=23%).

### Example 21

### 5-(2-chloro-6-fluorobenzyl)-2-methyl-4-((1-methylpiperidin-2-yl)methyl)-2,4-dihydro-3H-1,2,4-triazol-3-one

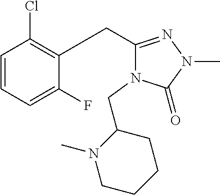 [SMILES: CN1CCCCC1Cn1c(Cc2c(F)cccc2Cl)nn(C)c1=O]

The title compound was prepared by the general procedure (scheme 1, step 4, method A) starting from intermediate 3 and bromide CAS: 1390654-84-8. It was purified by flash silica gel column chromatography using a linear gradient of methanol in dichloromethane from 0 to 20% (Y=44%).

### Example 22

### 5-(2-chloro-6-fluorobenzyl)-2-methyl-4-(2-morpholinoethyl)-2,4-dihydro-3H-1,2,4-triazol-3-one

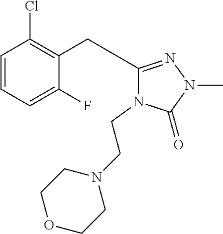 [SMILES: Cn1nc(Cc2c(F)cccc2Cl)n(CCN2CCOCC2)c1=O]

The title compound was prepared by the general procedure (scheme 1, step 4, method B) starting from intermediate 3 and bromide CAS: 42802-94-8. It was purified by HPLC (Y=20%).

### Example 23

### 4-((2,2-dimethylcyclohexyl)methyl)-2-methyl-5-(2-(trifluoromethyl)benzyl)-2,4-dihydro-3H-1,2,4-triazol-3-one

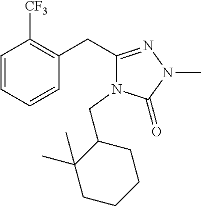 [SMILES: Cn1nc(Cc2ccccc2C(F)(F)F)n(CC2CCCCC2(C)C)c1=O]

The title compound was prepared by the general procedure (scheme 1, step 4, method A) starting from intermediate 12 and bromide CAS: 1501249-61-1. It was purified by HPLC (Y=11%).

### Example 24

### 4-(2-chloro-6-fluorobenzyl)-5-(cyclohexylmethyl)-2-methyl-2,4-dihydro-3H-1,2,4-triazol-3-one

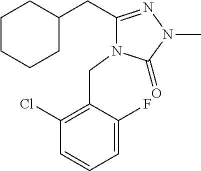 [SMILES: Cn1nc(CC2CCCCC2)n(Cc2c(F)cccc2Cl)c1=O]

The title compound was prepared by the general procedure (scheme 1, step 4, method B) starting from intermediate 9 and chloride CAS: 55117-15-2. It was purified by flah silica gel column chromatography using a linear gradient of ethyl acetate in hexane (with 1% TEA) from 0% to 100% (Y=50%).

### Example 25

### 5-(cyclohexylmethyl)-2-methyl-4-(2-(trifluoromethyl)benzyl)-2,4-dihydro-3H-1,2,4-triazol-3-one

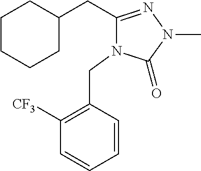 [SMILES: Cn1nc(CC2CCCCC2)n(Cc2ccccc2C(F)(F)F)c1=O]

The title compound was prepared by the general procedure (scheme 1, step 4, method B) starting from intermediate 9 and chloride CAS: 21742-00-7. It was purified by flah silica gel column chromatography using a linear gradient of ethyl acetate in hexane (with 1% TEA) from 0% to 100% (Y=17%).

### Example 26

### 4-((4,4-difluorocyclohexyl)methyl)-5-(2-fluorobenzyl)-2-methyl-2,4-dihydro-3H-1,2,4-triazol-3-one

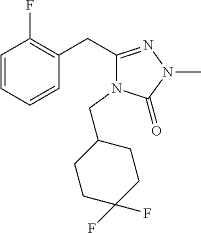 [SMILES: Cn1nc(Cc2ccccc2F)n(CC2CCC(F)(F)CC2)c1=O]

ethyl 2-(2-fluorophenyl)acetimidate hydrochloride (intermediate 13). The title compound was prepared by the general procedure (scheme 1, step 1) starting from commercially available nitrile CAS: 326-62-5.

ethyl-N-(ethoxycarbonyl)-2-(2-fluorophenyl)acetimidate (intermediate 14). The title compound was prepared by the general procedure (scheme 1, step 2) starting from intermediate 13. It was purified by flash silica gel column chromatography using a linear gradient of ethyl acetate in hexane from 0 to 50% (Y=63%).

5-(2-fluorobenzyl)-2-methyl-2,4-dihydro-3H-1,2,4-triazol-3-one (intermediate 15). The title compound was prepared by the general procedure (scheme 1, step 3) starting from intermediate 14 and methyl hydrazine CAS: 60-34-4 It was purified by flash silica gel column chromatography using a linear gradient of ethyl acetate in hexane from 0 to 100% (Y=17%).

4-((4,4-difluorocyclohexyl)methyl)-5-(2-fluorobenzyl)-2-methyl-2,4-dihydro-3H-1,2,4-triazol-3-one. The title compound was prepared by the general procedure (scheme 1, step 4, method A) starting from intermediate 15 and bromide CAS: 858121-94-5. It was purified by flah silica gel column chromatography using a linear gradient of ethyl acetate in hexane (with 1% TEA) from 0% to 100% (Y=38%).

### Example 27

### 4-(cyclohexylmethyl)-5-(2-fluorobenzyl)-2-methyl-2,4-dihydro-3H-1,2,4-triazol-3-one

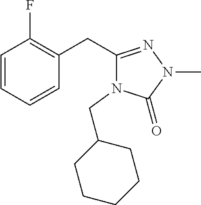 [SMILES: Cn1nc(Cc2ccccc2F)n(CC2CCCCC2)c1=O]

The title compound was prepared by the general procedure (scheme 1, step 4, method A) starting from intermediate 15 and bromide CAS: 2550-36-9. It was purified by flah silica gel column chromatography using a linear gradient of ethyl acetate in hexane (with 1% TEA) from 0% to 100% (Y=41%).

### Example 28

### 4-(cycloheptylmethyl)-2-methyl-5-(2-(trifluoromethyl)benzyl)-2,4-dihydro-3H-1,2,4-triazol-3-one

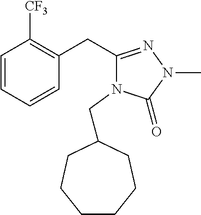 [SMILES: Cn1nc(Cc2ccccc2C(F)(F)F)n(CC2CCCCCC2)c1=O]

The title compound was prepared by the general procedure (scheme 1, step 4, method A) starting from intermediate 12 and bromide CAS: 3814-32-2. It was purified by HPLC (Y=10%).

### Example 29

### 4-(2-chloro-5-(5-fluoropyrimidin-2-yl)benzyl)-5-(4-fluorobenzyl)-2-methyl-2,4-dihydro-3H-1,2,4-triazol-3-one

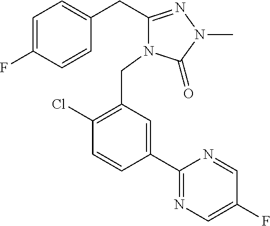 [SMILES: Cn1nc(Cc2ccc(F)cc2)n(Cc2cc(-c3ncc(F)cn3)ccc2Cl)c1=O]

ethyl 2-(4-fluorophenyl)acetimidate hydrochloride (intermediate 16). The title compound was prepared by the general procedure (scheme 1, step 1) starting from commercially available nitrile CAS: 459-22-3.

ethyl-N-(ethoxycarbonyl)-2-(4-fluorophenyl)acetimidate (intermediate 17). The title compound was prepared by the general procedure (scheme 1, step 2) starting from intermediate 16. It was purified by flash silica gel column chromatography using a linear gradient of ethyl acetate in hexane from 0 to 50% (Y=57%).

5-(4-fluorobenzyl)-2-methyl-2,4-dihydro-3H-1,2,4-triazol-3-one (intermediate 18). The title compound was prepared by the general procedure (scheme 1, step 3) starting from intermediate 17 and methyl hydrazine CAS: 60-34-4 It was purified by flash silica gel column chromatography using a linear gradient of ethyl acetate in hexane from 0 to 100% (Y=23%).

methyl 2-chloro-5-(5-fluoropyrimidin-2-yl)benzoate (intermediate 19). The title compound was prepared by the general procedure (scheme 2, step 5) starting from benzoic acid derivative CAS: 1227807-75-1. It was used for the next step without further purification.

(2-chloro-5-(5-fluoropyrimidin-2-yl)phenyl)methanol (intermediate 20). The title compound was prepared by the general procedure (scheme 2, step 6) starting from intermediate 19. It was purified by flash silica gel column chromatography using a linear gradient of ethyl acetate in hexane from 0 to 50% (Y=49%).

2-(4-chloro-3-(chloromethyl)phenyl)-5-fluoropyrimidine (intermediate 21). The title compound was prepared by the general procedure (scheme 2, step 7) starting from intermediate 20.

4-(2-chloro-5-(5-fluoropyrimidin-2-yl)benzyl)-5-(4-fluorobenzyl)-2-methyl-2,4-dihydro-3H-1,2,4-triazol-3-one. The title compound was prepared by the general procedure (scheme 1, step 4, method B) starting from intermediate 18 and chloride (intermediate 21). It was purified by flah silica gel column chromatography using a linear gradient of ethyl acetate in hexane (with 1% TEA) from 0% to 100% (Y=25%).

### Example 30

### 5-(2-chloro-6-fluorobenzyl)-4-(2-(3,3-dimethylmorpholino)ethyl)-2-methyl-2,4-dihydro-3H-1,2,4-triazol-3-one

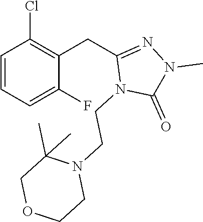 [SMILES: Cn1nc(Cc2c(F)cccc2Cl)n(CCN2CCOCC2(C)C)c1=O]

The title compound was prepared by the general procedure (scheme 1, step 4, method B) starting from intermediate 3 and bromide CAS: 1098202-59-5. It was purified by HPLC (Y=20%).

### Example 31

### 5-(cyclohexylmethyl)-2-methyl-4-((2-methylpyridin-3-yl)methyl)-2,4-dihydro-3H-1,2,4-triazol-3-one

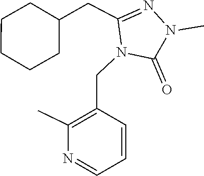 [SMILES: Cc1ncccc1Cn1c(CC2CCCCC2)nn(C)c1=O]

The title compound was prepared by the general procedure (scheme 1, step 4, method B) starting from intermediate 9 and chloride CAS: 120277-68-1. It was purified by HPLC (Y=28%).

### Example 32

### 4-((2,2-dimethylcyclohexyl)methyl)-5-(2-fluorobenzyl)-2-methyl-2,4-dihydro-3H-1,2,4-triazol-3-one

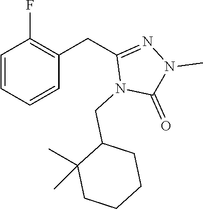 [SMILES: Cn1nc(Cc2ccccc2F)n(CC2CCCCC2(C)C)c1=O]

The title compound was prepared by the general procedure (scheme 1, step 4, method A) starting from intermediate 15 and bromide CAS: 1501249-61-1. It was purified by flah silica gel column chromatography using a linear gradient of ethyl acetate in hexane (with 1% TEA) from 0% to 100% (Y=13%).

### Example 33

### 5-(2-chloro-6-fluorobenzyl)-4-((2,2-dimethylcyclohexyl)methyl)-2-methyl-2,4-dihydro-3H-1,2,4-triazol-3-one

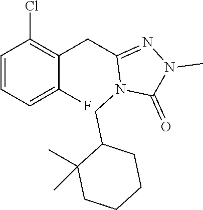 [SMILES: Cn1nc(Cc2c(F)cccc2Cl)n(CC2CCCCC2(C)C)c1=O]

The title compound was prepared by the general procedure (scheme 1, step 4, method A) starting from intermediate 3 and bromide CAS: 1501249-61-1. It was purified by HPLC (Y=16%).

### Example 34

### 5-(2-chloro-6-fluorobenzyl)-4-((4,4-difluorocyclohexyl)methyl)-2-ethyl-2,4-dihydro-3H-1,2,4-triazol-3-one

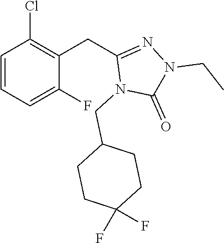 [SMILES: CCn1nc(Cc2c(F)cccc2Cl)n(CC2CCC(F)(F)CC2)c1=O]

5-[(2-chloro-6-fluorophenyl)methyl]-2-ethyl-2,4-dihydro-3H-1,2,4-triazol-3-one (intermediate 27). The title compound was prepared by the general procedure (scheme 1, step 3) starting from intermediate 2 and ethylhydrazine oxalate (1:1) CAS: 6629-60-3. (Y=50%).

5-[(2-chloro-6-fluorophenyl)methyl]-4-[(4,4-difluorocyclohexyl)methyl]-2-ethyl-2,4-dihydro-3H-1,2,4-triazol-3-one. The title compound was prepared by the general procedure (scheme 1, step 4, method A) starting from intermediate 27 and bromide CAS: 858121-94-5. It was purified by HPLC (Y=31%).

### Example 35

### 5-(2-chloro-4-fluorobenzyl)-2-methyl-4-(spiro[2.5]octan-6-ylmethyl)-2,4-dihydro-3H-1,2,4-triazol-3-one

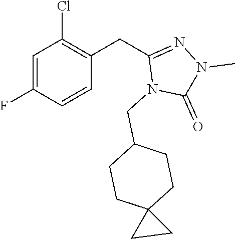 [SMILES: Cn1nc(Cc2ccc(F)cc2Cl)n(CC2CCC3(CC2)CC3)c1=O]

The title compound was prepared by the general procedure (scheme 1, step 4, method A) starting from intermediate 6 and bromide CAS: 1621225-50-0. It was purified by HPLC (Y=33%).

### Example 36

### 5-(2-chloro-4-fluorobenzyl)-4-(2-(4,4-difluorocyclohexyl)ethyl)-2-methyl-2,4-dihydro-3H-1,2,4-triazol-3-one

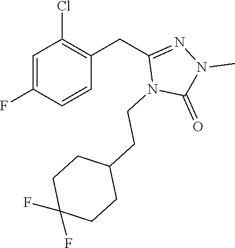 [SMILES: Cn1nc(Cc2ccc(F)cc2Cl)n(CCC2CCC(F)(F)CC2)c1=O]

The title compound was prepared by the general procedure (scheme 1, step 4, method A) starting from intermediate 6 and bromide CAS: 2092565-10-9. It was purified by HPLC (Y=39%).

### Example 37

### 4-(cycloheptylmethyl)-5-(2-fluorobenzyl)-2-methyl-2,4-dihydro-3H-1,2,4-triazol-3-one

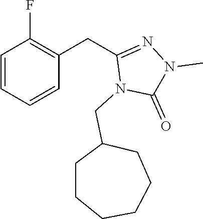 [SMILES: Cn1nc(Cc2ccccc2F)n(CC2CCCCCC2)c1=O]

The title compound was prepared by the general procedure (scheme 1, step 4, method A) starting from intermediate 15 and bromide CAS: 3814-32-2. It was purified by HPLC (Y=17%).

### Example 38

### 4-(2-chloro-5-(5-fluoropyrimidin-2-yl)benzyl)-5-(cyclohexylmethyl)-2-methyl-2,4-dihydro-3H-1,2,4-triazol-3-one

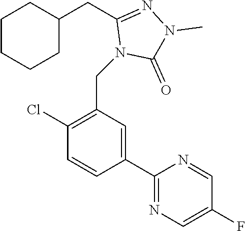 [SMILES: Cn1nc(CC2CCCCC2)n(Cc2cc(-c3ncc(F)cn3)ccc2Cl)c1=O]

The title compound was prepared by the general procedure (scheme 1, step 4, method B) starting from intermediate 9 and chloride (intermediate 21). It was purified by flah silica gel column chromatography using a linear gradient of ethyl acetate in hexane (with 1% TEA) from 0% to 100% (Y=48%).

### Example 39

### 4-(2-(4,4-difluorocyclohexyl)ethyl)-2-methyl-5-(2-(trifluoromethyl)benzyl)-2,4-dihydro-3H-1,2,4-triazol-3-one

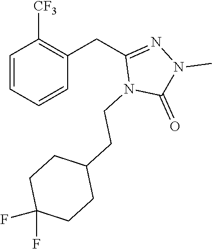 [SMILES: Cn1nc(Cc2ccccc2C(F)(F)F)n(CCC2CCC(F)(F)CC2)c1=O]

The title compound was prepared by the general procedure (scheme 1, step 4, method A) starting from intermediate 12 and bromide CAS: 2092565-10-9. It was purified by flah silica gel column chromatography using a linear gradient of ethyl acetate in hexane (with 1% TEA) from 0% to 100% (Y=33%).

### Example 40

### 2-methyl-5-(2-(trifluoromethyl)benzyl)-4-((4-(trifluoromethyl)cyclohexyl)methyl)-2,4-dihydro-3H-1,2,4-triazol-3-one

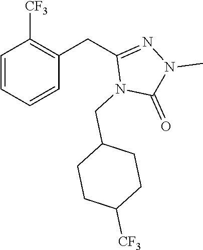 [SMILES: Cn1nc(Cc2ccccc2C(F)(F)F)n(CC2CCC(C(F)(F)F)CC2)c1=O]

The title compound was prepared by the general procedure (scheme 1, step 4, method A) starting from intermediate 12 and bromide CAS: 858121-96-7. It was purified by HPLC (Y=10%).

### Example 41

### 5-(2-chloro-4-fluorobenzyl)-4-(2-(4,4-dimethylcyclohexyl)ethyl)-2-methyl-2,4-dihydro-3H-1,2,4-triazol-3-one

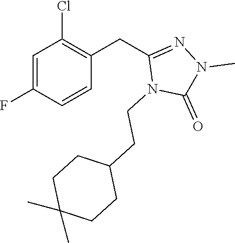 [SMILES: Cn1nc(Cc2ccc(F)cc2Cl)n(CCC2CCC(C)(C)CC2)c1=O]

The title compound was prepared by the general procedure (scheme 1, step 4, method A) starting from intermediate 6 and bromide CAS: 570398-26-4. It was purified by HPLC (Y=48%).

### Example 42

### 5-(2-chlorobenzyl)-4-((4,4-difluorocyclohexyl)methyl)-2-methyl-2,4-dihydro-3H-1,2,4-triazol-3-one

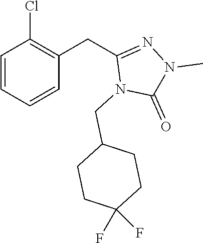 [SMILES: Cn1nc(Cc2ccccc2Cl)n(CC2CCC(F)(F)CC2)c1=O]

ethyl 2-(2-chlorophenyl)acetimidate hydrochloride (intermediate 22). The title compound was prepared by the general procedure (scheme 1, step 1) starting from commercially available nitrile CAS: 2856-63-5.

ethyl-2-(2-chlorophenyl)-N-(ethoxycarbonyl)acetimidate (intermediate 23). The title compound was prepared by the general procedure (scheme 1, step 2) starting from intermediate 22. It was purified by flash silica gel column chromatography using a linear gradient of dichloromethane in hexane from 50 to 100% (Y=34%).

5-(2-chlorobenzyl)-2-methyl-2,4-dihydro-3H-1,2,4-triazol-3-one (intermediate 24). The title compound was prepared by the general procedure (scheme 1, step 3) starting from intermediate 23 and methyl hydrazine CAS: 60-34-4 It was purified by flash silica gel column chromatography using a linear gradient of ethyl acetate in hexane from 50 to 100% (Y=5%).

5-(2-chlorobenzyl)-4-((4,4-difluorocyclohexyl)methyl)-2-methyl-2,4-dihydro-3H-1,2,4-triazol-3-one. The title compound was prepared by the general procedure (scheme 1, step 4, method A) starting from intermediate 24 and bromide CAS: 858121-94-5. It was purified by HPLC (Y=43%).

### Example 43

### 5-(2-chloro-6-fluorobenzyl)-2-(cyclopropylmethyl)-4-((4,4-difluorocyclohexyl)methyl)-2,4-dihydro-3H-1,2,4-triazol-3-one

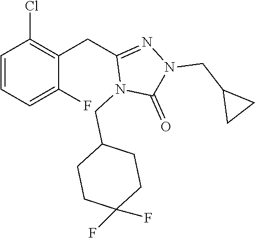 [SMILES: O=c1n(CC2CC2)nc(Cc2c(F)cccc2Cl)n1CC1CCC(F)(F)CC1]

5-(2-chloro-6-fluorobenzyl)-2-(cyclopropylmethyl)-2,4-dihydro-3H-1,2,4-triazol-3-one (intermediate 25). The title compound was prepared by the general procedure (scheme 1, step 3) starting from intermediate 2 and (cyclopropylmethyl) hydrazine CAS: 809282-61-9. (Y=62%).

5-(2-chloro-6-fluorobenzyl)-2-(cyclopropylmethyl)-4-((4,4-difluorocyclohexyl)methyl)-2,4-dihydro-3H-1,2,4-triazol-3-one. The title compound was prepared by the general procedure (scheme 1, step 4, method A) starting from intermediate 25 and bromide CAS: 858121-94-5. It was purified by HPLC (Y=75%).

### Example 44

### 5-(2-chloro-6-fluorobenzyl)-4-((4,4-difluorocyclohexyl)methyl)-2-(2,2,2-trifluoroethyl)-2,4-dihydro-3H-1,2,4-triazol-3-one

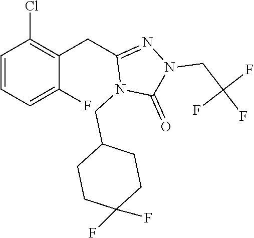 [SMILES: O=c1n(CC(F)(F)F)nc(Cc2c(F)cccc2Cl)n1CC1CCC(F)(F)CC1]

5-(2-chloro-6-fluorobenzyl)-2-(2,2,2-trifluoroethyl)-2,4-dihydro-3H-1,2,4-triazol-3-one (intermediate 26). The title compound was prepared by the general procedure (scheme 1, step 3) starting from intermediate 2 and (2,2,2-trifluoroethyl) hydrazine CAS: 5042-30-8. (Y=64%).

5-(2-chloro-6-fluorobenzyl)-4-((4,4-difluorocyclohexyl)methyl)-2-(2,2,2-trifluoroethyl)-2,4-dihydro-3H-1,2,4-triazol-3-one. The title compound was prepared by the general procedure (scheme 1, step 4, method A) starting from intermediate 26 and bromide CAS: 858121-94-5. It was purified by HPLC (Y=48%).

### Example 45

### 4-(2-(4,4-dimethylcyclohexyl)ethyl)-5-(2-fluorobenzyl)-2-methyl-2,4-dihydro-3H-1,2,4-triazol-3-one

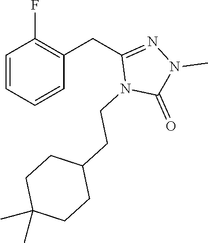

The title compound was prepared by the general procedure (scheme 1, step 4, method A) starting from intermediate 15 and bromide CAS: 570398-26-4. It was purified by HPLC (Y=19%).

### Example 46

### 4-(2-(4,4-difluorocyclohexyl)ethyl)-5-(2-fluorobenzyl)-2-methyl-2,4-dihydro-3H-1,2,4-triazol-3-one

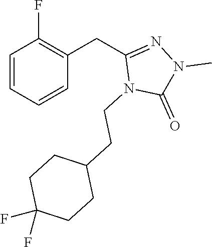 [SMILES: Cn1nc(Cc2ccccc2F)n(CCC2CCC(F)(F)CC2)c1=O]

The title compound was prepared by the general procedure (scheme 1, step 4, method A) starting from intermediate 15 and bromide CAS: 2092565-10-9. It was purified by HPLC (Y=8%).

### Example 47

### 5-(2-fluorobenzyl)-2-methyl-4-(spiro[2.5]octan-6-ylmethyl)-2,4-dihydro-3H-1,2,4-triazol-3-one

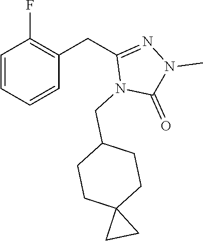 [SMILES: Cn1nc(Cc2ccccc2F)n(CC2CCC3(CC2)CC3)c1=O]

The title compound was prepared by the general procedure (scheme 1, step 4, method A) starting from intermediate 15 and bromide CAS: 1621225-50-0. It was purified by HPLC (Y=9%).

### Example 48

### 2-methyl-4-(spiro[2.5]octan-6-ylmethyl)-5-(2-(trifluoromethyl)benzyl)-2,4-dihydro-3H-1,2,4-triazol-3-one

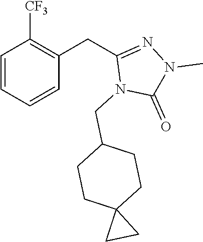 [SMILES: Cn1nc(Cc2ccccc2C(F)(F)F)n(CC2CCC3(CC2)CC3)c1=O]

The title compound was prepared by the general procedure (scheme 1, step 4, method A) starting from intermediate 12 and bromide CAS: 1621225-50-0. It was purified by HPLC (Y=43%).

### Example 49

### 4-(2-(4,4-dimethylcyclohexyl)ethyl)-2-methyl-5-(2-(trifluoromethyl)benzyl)-2,4-dihydro-3H-1,2,4-triazol-3-one

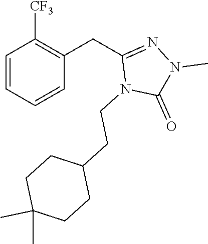

The title compound was prepared by the general procedure (scheme 1, step 4, method A) starting from intermediate 12 and bromide CAS: 570398-26-4. It was purified by HPLC (Y=31%).

### Example 50

### 2-benzyl-5-(2-chloro-6-fluorobenzyl)-4-((4,4-difluorocyclohexyl)methyl)-2,4-dihydro-3H-1,2,4-triazol-3-one

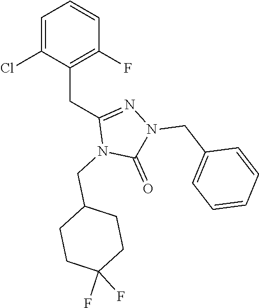 [SMILES: O=c1n(Cc2ccccc2)nc(Cc2c(F)cccc2Cl)n1CC1CCC(F)(F)CC1]

2-benzyl-5-(2-chloro-6-fluorobenzyl)-2,4-dihydro-3H-1,2,4-triazol-3-one (intermediate 28). The title compound was prepared by the general procedure (scheme 1, step 3) starting from intermediate 2 and benzylhydrazine dihydrochloride CAS: 20570-96-1. It was purified by flash silica gel column chromatography using a linear gradient of ethyl acetate in cyclohexane from 0% to 50% (Y=9%).

2-benzyl-5-(2-chloro-6-fluorobenzyl)-4-((4,4-difluorocyclohexyl)methyl)-2,4-dihydro-3H-1,2,4-triazol-3-one. The title compound was prepared by the general procedure (scheme 1, step 4, method B) starting from intermediate 28 and bromide CAS: 858121-94-5. It was purified by HPLC (Y=5%).

### Example 51

### 5-(2-fluorobenzyl)-2-methyl-4-((4-(trifluoromethyl)cyclohexyl)methyl)-2,4-dihydro-3H-1,2,4-triazol-3-one

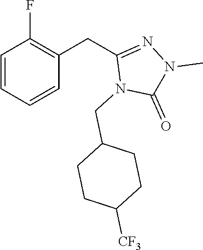 [SMILES: Cn1nc(Cc2ccccc2F)n(CC2CCC(C(F)(F)F)CC2)c1=O]

The title compound was prepared by the general procedure (scheme 1, step 4, method A) starting from intermediate 15 and bromide CAS: 858121-96-7. It was purified by HPLC (Y=24%).

### Example 52

### 5-(2-chloro-4-fluorobenzyl)-2-methyl-4-((4-(trifluoromethyl)cyclohexyl)methyl)-2,4-dihydro-3H-1,2,4-triazol-3-one

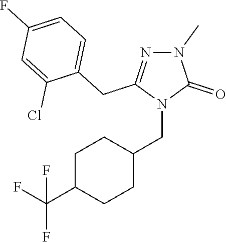 [SMILES: Cn1nc(Cc2ccc(F)cc2Cl)n(CC2CCC(C(F)(F)F)CC2)c1=O]

The title compound was prepared by the general procedure (scheme 1, step 4, method A) starting from intermediate 6 and bromide CAS: 858121-96-7. It was purified by HPLC (Y=13%).

### Example 53

### 5-(2-chloro-6-fluorobenzyl)-4-(2-hydroxyethyl)-2-methyl-2,4-dihydro-3H-1,2,4-triazol-3-one

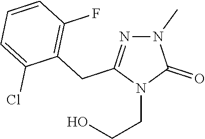 [SMILES: Cn1nc(Cc2c(F)cccc2Cl)n(CCO)c1=O]

The title compound was prepared by the general procedure (scheme 1, step 4, method A) starting from intermediate 3 and bromide CAS: 540-51-2. It was purified by HPLC (Y=35%).

### Example 54

### 5-[(2-chlorophenyl)methyl]-4-(cycloheptylmethyl)-2-(2,2,2-trifluoroethyl)-2,4-dihydro-3H-1,2,4-triazol-3-one

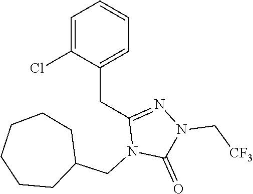 [SMILES: O=c1n(CC(F)(F)F)nc(Cc2ccccc2Cl)n1CC1CCCCCC1]

5-[(2-chlorophenyl)methyl]-2-(2,2,2-trifluoroethyl)-2,4-dihydro-3H-1,2,4-triazol-3-one (intermediate 29). The title compound was prepared by the general procedure (scheme 1, step 3) starting from intermediate 23 and (2,2,2-trifluoroethyl)hydrazine CAS: 540-51-2. The product was used without purification in the next step (Y=41%).

5-[(2-chlorophenyl)methyl]-4-(cycloheptylmethyl)-2-(2,2,2-trifluoroethyl)-2,4-dihydro-3H-1,2,4-triazol-3-one. The title compound was prepared by the general procedure (scheme 1, step 4, method A) starting from intermediate 29 and bromide CAS: 3814-32-2. It was purified by HPLC (Y=16%).

### Example 55

### 5-[(2-chlorophenyl)methyl]-4-(cycloheptylmethyl)-2-methyl-2,4-dihydro-3H-1,2,4-triazol-3-one

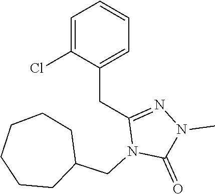 [SMILES: Cn1nc(Cc2ccccc2Cl)n(CC2CCCCCC2)c1=O]

The title compound was prepared by the general procedure (scheme 1, step 4, method A) starting from intermediate 24 and bromide CAS: 3814-32-2. It was purified by HPLC (Y=20%).

### Example 56

### 5-[(2-chloro-6-fluorophenyl)methyl]-2-methyl-4-[(1-methylcycloheptyl)methyl]-2,4-dihydro-3H-1,2,4-triazol-3-one

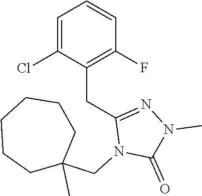 [SMILES: Cn1nc(Cc2c(F)cccc2Cl)n(CC2(C)CCCCCC2)c1=O]

The title compound was prepared by the general procedure (scheme 1, step 4, method A) starting from intermediate 3 and bromide CAS: 1936250-61-1. It was purified by HPLC (Y=15%).

### Example 57

### 5-[(2-chloro-6-fluorophenyl)methyl]-4-[(4,4-difluorocyclohexyl)methyl]-2-(3,3,3-trifluoropropyl)-2,4-dihydro-3H-1,2,4-triazol-3-one

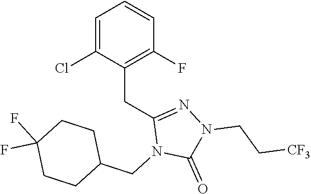 [SMILES: O=c1n(CCC(F)(F)F)nc(Cc2c(F)cccc2Cl)n1CC1CCC(F)(F)CC1]

5-[(2-chloro-6-fluorophenyl)methyl]-2-(3,3,3-trifluoropropyl)-2,4-dihydro-3H-1,2,4-triazol-3-one (intermediate 30). The title compound was prepared by the general procedure (scheme 1, step 3) starting from intermediate 2 and (3,3,3-trifluoropropyl)hydrazine CAS: 1446322-01-5 The product was used without purification in the next step (Y=67%). 5-[(2-chloro-6-fluorophenyl)methyl]-4-[(4,4-difluorocyclohexyl)methyl]-2-(3,3,3-trifluoropropyl)-2,4-dihydro-3H-1,2,4-triazol-3-one. The title compound was prepared by the general procedure (scheme 1, step 4, method A) starting from intermediate 30 and bromide CAS: 858121-94-5. It was purified by HPLC (Y=56%).

### Example 58

### 5-[(2-chloro-6-fluorophenyl)methyl]-4-[(4,4-difluorocyclohexyl)methyl]-2-(2,2-difluoroethyl)-2,4-dihydro-3H-1,2,4-triazol-3-one

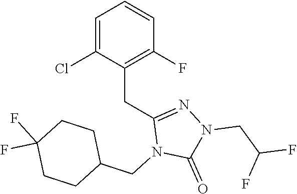 [SMILES: O=c1n(CC(F)F)nc(Cc2c(F)cccc2Cl)n1CC1CCC(F)(F)CC1]

5-[(2-chloro-6-fluorophenyl)methyl]-2-(2,2-difluoroethyl)-2,4-dihydro-3H-1,2,4-triazol-3-one (intermediate 31). The title compound was prepared by the general procedure (scheme 1, step 3) starting from intermediate 2 and (2,2-difluoroethyl)hydrazine CAS: 1504582-53-9 The product was used without purification in the next step (Y=78%). 5-[(2-chloro-6-fluorophenyl)methyl]-4-[(4,4-difluorocyclohexyl)methyl]-2-(3,3,3-trifluoropropyl)-2,4-dihydro-3H-1,2,4-triazol-3-one. The title compound was prepared by the general procedure (scheme 1, step 4, method A) starting from intermediate 31 and bromide CAS: 858121-94-5. It was purified by HPLC (Y=9%).

### Example 59

### 4-[(2-chloro-6-fluorophenyl)methyl]-5-(cycloheptylmethyl)-2-methyl-2,4-dihydro-3H-1,2,4-triazol-3-one

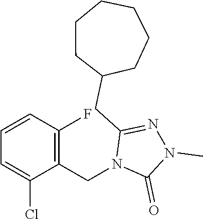 [SMILES: Cn1nc(CC2CCCCCC2)n(Cc2c(F)cccc2Cl)c1=O]

ethyl 2-cycloheptylethanimidate hydrochloride (intermediate 32). The title compound was prepared by the general procedure (scheme 1, step 1) starting from commercially available nitrile CAS: 5452-65-3.

ethyl-2-cycloheptyl-N-(ethoxycarbonyl)ethanimidate (intermediate 33). The title compound was prepared by the general procedure (scheme 1, step 2) starting from intermediate 32. It was purified by flash silica gel column chromatography using Hexane/EtOAc 4:1 v/v as the eluent (Y=71%).

5-(cycloheptylmethyl)-2-methyl-2,4-dihydro-3H-1,2,4-triazol-3-one (intermediate 34). The title compound was prepared by the general procedure (scheme 1, step 3) starting from intermediate 33 and methyl hydrazine CAS: 302-15-8. It was used without purification in the next step (Y=90%).

4-[(2-chloro-6-fluorophenyl)methyl]-5-(cycloheptylmethyl)-2-methyl-2,4-dihydro-3H-1,2,4-triazol-3-one. The title compound was prepared by the general procedure (scheme 1, step 4, method B) starting from intermediate 34 and chloride CAS: 55117-15-2. It was purified by HPLC (Y=60%).

### Example 60

### 4-[(2-chloro-6-fluorophenyl)methyl]-5-(cycloheptylmethyl)-2-ethyl-2,4-dihydro-3H-1,2,4-triazol-3-one

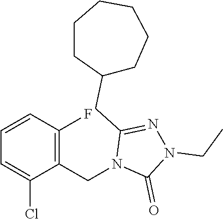 [SMILES: CCn1nc(CC2CCCCCC2)n(Cc2c(F)cccc2Cl)c1=O]

5-(cycloheptylmethyl)-2-ethyl-2,4-dihydro-3H-1,2,4-triazol-3-one (intermediate 35). The title compound was prepared by the general procedure (scheme 1, step 3) starting from intermediate 33 and ethyl hydrazine CAS: 6629-60-3. It was used without purification in the next step (Y=90%).

4-[(2-chloro-6-fluorophenyl)methyl]-5-(cycloheptylmethyl)-2-methyl-2,4-dihydro-3H-1,2,4-triazol-3-one. The title compound was prepared by the general procedure (scheme 1, step 4, method A) starting from intermediate 35 and chloride CAS: 55117-15-2. It was purified by HPLC (Y=73%).

### Example 61

### 4-[(2-chloro-6-fluorophenyl)methyl]-5-(cycloheptylmethyl)-2-(2,2,2-trifluoroethyl)-2,4-dihydro-3H-1,2,4-triazol-3-one

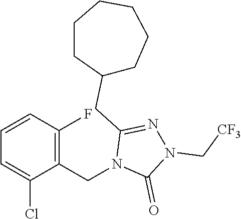 [SMILES: O=c1n(CC(F)(F)F)nc(CC2CCCCCC2)n1Cc1c(F)cccc1Cl]

5-(cycloheptylmethyl)-2-(2,2,2-trifluoroethyl)-2,4-dihydro-3H-1,2,4-triazol-3-one (intermediate 36). The title compound was prepared by the general procedure (scheme 1, step 3) starting from intermediate 33 and (2,2,2-trifluoroethyl)hydrazine CAS: 5042-30-8. It was used without purification in the next step (Y=80%).

4-[(2-chloro-6-fluorophenyl)methyl]-5-(cycloheptylmethyl)-2-(2,2,2-trifluoroethyl)-2,4-dihydro-3H-1,2,4-triazol-3-one. The title compound was prepared by the general procedure (scheme 1, step 4, method A) starting from intermediate 36 and chloride CAS: 55117-15-2. It was purified by HPLC (Y=88%).

### Example 62

### 5-[(2-chlorophenyl)methyl]-4-[(4,4-difluorocyclohexyl)methyl]-2-(2,2,2-trifluoroethyl)-2,4-dihydro-3H-1,2,4-triazol-3-one

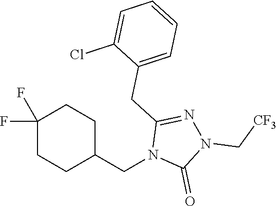 [SMILES: O=c1n(CC(F)(F)F)nc(Cc2ccccc2Cl)n1CC1CCC(F)(F)CC1]

5-[(2-chlorophenyl)methyl]-4-[(4,4-difluorocyclohexyl)methyl]-2-(2,2,2-trifluoroethyl)-2,4-dihydro-3H-1,2,4-triazol-3-one. The title compound was prepared by the general procedure (scheme 1, step 4, method A) starting from intermediate 29 and bromide CAS: 858121-94-5. It was purified by HPLC (Y=12%).

### Example 63

### 5-[(2-chlorophenyl)methyl]-4-[(4,4-difluorocyclohexyl)methyl]-2-ethyl-2,4-dihydro-3H-1,2,4-triazol-3-one

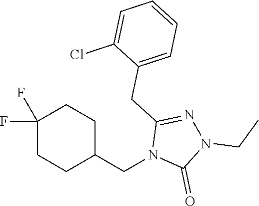 [SMILES: CCn1nc(Cc2ccccc2Cl)n(CC2CCC(F)(F)CC2)c1=O]

5-[(2-chlorophenyl)methyl]-2-ethyl-2,4-dihydro-3H-1,2,4-triazol-3-one (intermediate 37). The title compound was prepared by the general procedure (scheme 1, step 3) starting from intermediate 23 and ethyl hydrazine CAS: 6629-60-3. It was used without purification in the next step (Y=38%).

5-[(2-chlorophenyl)methyl]-4-[(4,4-difluorocyclohexyl)methyl]-2-ethyl-2,4-dihydro-3H-1,2,4-triazol-3-one. The title compound was prepared by the general procedure (scheme 1, step 4, method A) starting from intermediate 37 and bromide CAS: 858121-94-5. It was purified by HPLC (Y=6%).

### Example 64

### 5-[(2-chlorophenyl)methyl]-4-(cycloheptylmethyl)-2-ethyl-2,4-dihydro-3H-1,2,4-triazol-3-one

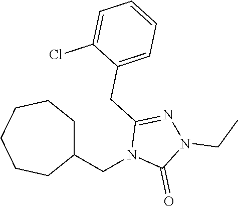 [SMILES: CCn1nc(Cc2ccccc2Cl)n(CC2CCCCCC2)c1=O]

The title compound was prepared by the general procedure (scheme 1, step 4, method A) starting from intermediate 37 and bromide CAS: 3814-32-2. It was purified by HPLC (Y=7%).

### Example 65

### 5-[(2-chloro-6-fluorophenyl)methyl]-4-[(3,3-difluorocyclopentyl)methyl]-2-ethyl-2,4-dihydro-3H-1,2,4-triazol-3-one

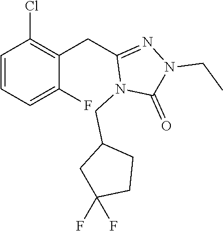 [SMILES: CCn1nc(Cc2c(F)cccc2Cl)n(CC2CCC(F)(F)C2)c1=O]

The title compound was prepared by the general procedure (scheme 1, step 4, method A) starting from intermediate 27 and bromide CAS: 1695914-13-6. It was purified by HPLC (Y=75%).

### Example 66

### 5-[(2-chloro-6-fluorophenyl)methyl]-2-(cyclopropylmethyl)-4-[(3,3-difluorocyclopentyl)methyl]-2,4-dihydro-3H-1,2,4-triazol-3-one

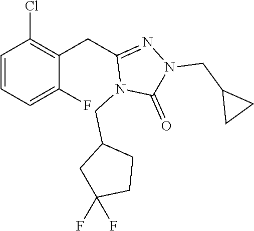 [SMILES: O=c1n(CC2CC2)nc(Cc2c(F)cccc2Cl)n1CC1CCC(F)(F)C1]

The title compound was prepared by the general procedure (scheme 1, step 4, method A) starting from intermediate 25 and bromide CAS: 1695914-13-6. It was purified by HPLC (Y=80%).

### Example 67

### 5-[(2-chloro-6-fluorophenyl)methyl]-4-(cycloheptylmethyl)-2-(2,2,2-trifluoroethyl)-2,4-dihydro-3H-1,2,4-triazol-3-one

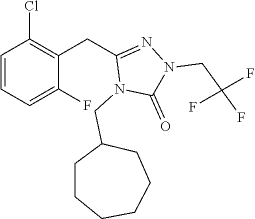 [SMILES: O=c1n(CC(F)(F)F)nc(Cc2c(F)cccc2Cl)n1CC1CCCCCC1]

The title compound was prepared by the general procedure (scheme 1, step 4, method A) starting from intermediate 26 and bromide CAS: 3814-32-2. It was purified by HPLC (Y=99%).

### Example 68

### 5-[(2-chloro-6-fluorophenyl)methyl]-4-[(1-methylpiperidin-2-yl)methyl]-2-(2,2,2-trifluoroethyl)-2,4-dihydro-3H-1,2,4-triazol-3-one

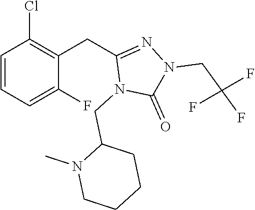 [SMILES: CN1CCCCC1Cn1c(Cc2c(F)cccc2Cl)nn(CC(F)(F)F)c1=O]

The title compound was prepared by the general procedure (scheme 1, step 4, method A) starting from intermediate 26 and bromide CAS: 1390654-84-8. It was purified by HPLC (Y=71%).

### Example 69

### 5-(2-chloro-6-fluorobenzyl)-2-(cyclopropylmethyl)-4-((4-(trifluoromethyl)cyclohexyl)methyl)-2,4-dihydro-3H-1,2,4-triazol-3-one

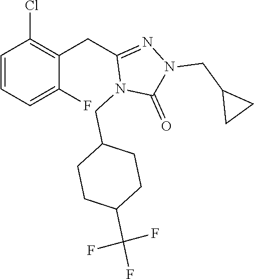 [SMILES: O=c1n(CC2CC2)nc(Cc2c(F)cccc2Cl)n1CC1CCC(C(F)(F)F)CC1]

The title compound was prepared by the general procedure (scheme 1, step 4, method A) starting from intermediate 25 and bromide CAS: 858121-96-7. It was purified by HPLC (Y=78%).

### Example 70

### 4-[(2-chloro-6-fluorophenyl)methyl]-5-[(4,4-difluorocyclohexyl)methyl]-2-(2,2,2-trifluoroethyl)-2,4-dihydro-3H-1,2,4-triazol-3-one

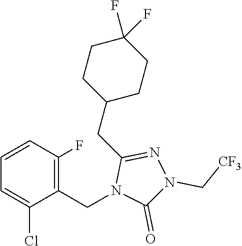 [SMILES: O=c1n(CC(F)(F)F)nc(CC2CCC(F)(F)CC2)n1Cc1c(F)cccc1Cl]

ethyl 2-(4,4-difluorocyclohexyl)ethanimidate hydrochloride (intermediate 38). The title compound was prepared by the general procedure (scheme 1, step 1) starting from commercially available nitrile CAS: 959600-88-5.

ethyl-2-(4,4-difluorocyclohexyl)-N-(ethoxycarbonyl)ethanimidate (intermediate 39). The title compound was prepared by the general procedure (scheme 1, step 2) starting from intermediate 38. It was purified by flash silica gel column chromatography using Hexane/EtOAc 4:1 v/v as the eluent (Y=45%).

5-[(4,4-difluorocyclohexyl)methyl]-2-(2,2,2-trifluoroethyl)-2,4-dihydro-3H-1,2,4-triazol-3-one (intermediate 40). The title compound was prepared by the general procedure (scheme 1, step 3) starting from intermediate 39 and (2,2,2-trifluoroethyl)hydrazine CAS: 5042-30-8. It was used without purification in the next step (Y=81%).

4-[(2-chloro-6-fluorophenyl)methyl]-5-[(4,4-difluorocyclohexyl)methyl]-2-(2,2,2-trifluoroethyl)-2,4-dihydro-3H-1,2,4-triazol-3-one. The title compound was prepared by the general procedure (scheme 1, step 4, method A) starting from intermediate 40 and chloride CAS: 55117-15-2. It was purified by HPLC (Y=11%).

### Example 71

### 4-[(2-chloro-6-fluorophenyl)methyl]-5-[(4,4-difluorocyclohexyl)methyl]-2-methyl-2,4-dihydro-3H-1,2,4-triazol-3-one

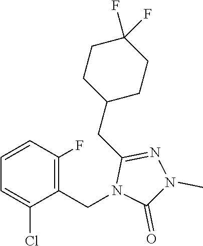 [SMILES: Cn1nc(CC2CCC(F)(F)CC2)n(Cc2c(F)cccc2Cl)c1=O]

5-[(4,4-difluorocyclohexyl)methyl]-2-methyl-2,4-dihydro-3H-1,2,4-triazol-3-one (intermediate 41). The title compound was prepared by the general procedure (scheme 1, step 3) starting from intermediate 39 and methyl hydrazine CAS: 302-15-8. It was used without purification in the next step (Y=79%).

4-[(2-chloro-6-fluorophenyl)methyl]-5-[(4,4-difluorocyclohexyl)methyl]-2-methyl-2,4-dihydro-3H-1,2,4-triazol-3-one. The title compound was prepared by the general procedure (scheme 1, step 4, method A) starting from intermediate 41 and chloride CAS: 55117-15-2. It was purified by HPLC (Y=8%).

Table 1 lists final compounds that were prepared according to the experimental procedure described for Example 1.

TABLE 1

| Example | Structure | IUPAC Name |
|---|---|---|
| 1 | 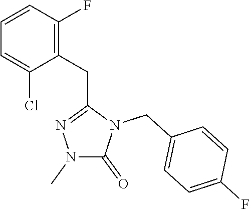 [SMILES: Cn1nc(Cc2c(F)cccc2Cl)n(Cc2ccc(F)cc2)c1=O] | 5-(2-chloro-6-fluorobenzyl)-4-(4- fluorobenzyl)-2-methyl-2,4-dihydro-3H- 1,2,4-triazol-3-one |
| 2 | 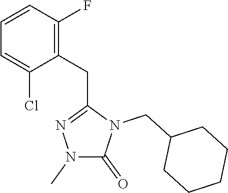 [SMILES: Cn1nc(Cc2c(F)cccc2Cl)n(CC2CCCCC2)c1=O] | 5-(2-chloro-6-fluorobenzyl)-4- (cyclohexylmethyl)-2-methyl-2,4-dihydro- 3H-1,2,4-triazol-3-one |
| 3 | 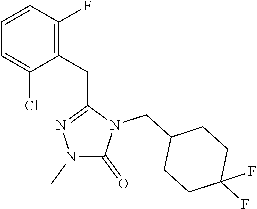 [SMILES: Cn1nc(Cc2c(F)cccc2Cl)n(CC2CCC(F)(F)CC2)c1=O] | 5-(2-chloro-6-fluorobenzyl)-4-((4,4- difluorocyclohexyl)methyl)-2-methyl-2,4- dihydro-3H-1,2,4-triazol-3-one |
| 4 | 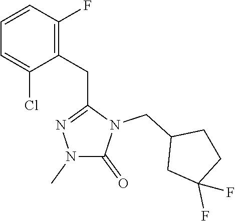 [SMILES: Cn1nc(Cc2c(F)cccc2Cl)n(CC2CCC(F)(F)C2)c1=O] | 5-(2-chloro-6-fluorobenzyl)-4-((3,3- difluorocyclopentyl)methyl)-2-methyl-2,4- dihydro-3H-1,2,4-triazol-3-one |
| 5 | 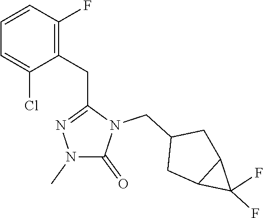 [SMILES: Cn1nc(Cc2c(F)cccc2Cl)n(CC2CC3C(C2)C3(F)F)c1=O] | 5-(2-chloro-6-fluorobenzyl)-4-((6,6- difluorobicyclo[3.1.0]hexan-3-yl)methyl)-2- methyl-2,4-dihydro-3H-1,2,4-triazol-3-one |
| 6 | 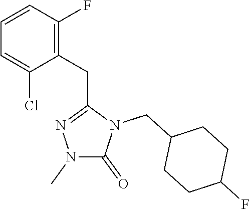 [SMILES: Cn1nc(Cc2c(F)cccc2Cl)n(CC2CCC(F)CC2)c1=O] | 5-(2-chloro-6-fluorobenzyl)-4-((4- fluorocyclohexyl)methyl)-2-methyl-2,4- dihydro-3H-1,2,4-triazol-3-one |
| 7 | 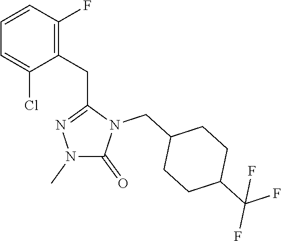 [SMILES: Cn1nc(Cc2c(F)cccc2Cl)n(CC2CCC(C(F)(F)F)CC2)c1=O] | 5-(2-chloro-6-fluorobenzyl)-2-methyl-4-((4- (trifluoromethyl)cyclohexyl)methyl)-2,4- dihydro-3H-1,2,4-triazol-3-one |
| 8 | 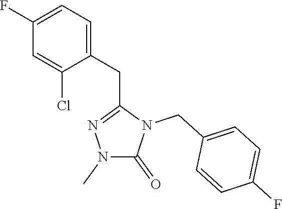 [SMILES: Cn1nc(Cc2ccc(F)cc2Cl)n(Cc2ccc(F)cc2)c1=O] | 5-(2-chloro-4-fluorobenzyl)-4-(4- fluorobenzyl)-2-methyl-2,4-dihydro-3H- 1,2,4-triazol-3-one |
| 9 | 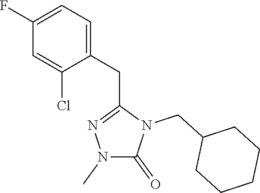 [SMILES: Cn1nc(Cc2ccc(F)cc2Cl)n(CC2CCCCC2)c1=O] | 5-(2-chloro-4-fluorobenzyl)-4- (cyclohexylmethyl)-2-methyl-2,4-dihydro- 3H-1,2,4-triazol-3-one |
| 10 | 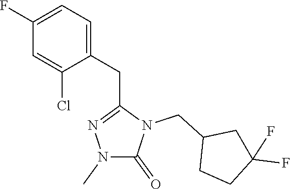 [SMILES: Cn1nc(Cc2ccc(F)cc2Cl)n(CC2CCC(F)(F)C2)c1=O] | 5-(2-chloro-4-fluorobenzyl)-4-((3,3- difluorocyclopentyl)methyl)-2-methyl-2,4- dihydro-3H-1,2,4-triazol-3-one |
| 11 | 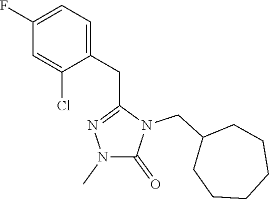 [SMILES: Cn1nc(Cc2ccc(F)cc2Cl)n(CC2CCCCCC2)c1=O] | 5-(2-chloro-4-fluorobenzyl)-4- (cycloheptylmethyl)-2-methyl-2,4-dihydro- 3H-1,2,4-triazol-3-one |
| 12 | 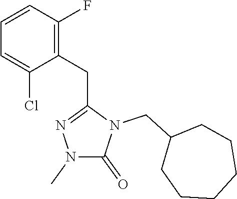 [SMILES: Cn1nc(Cc2c(F)cccc2Cl)n(CC2CCCCCC2)c1=O] | 5-(2-chloro-6-fluorobenzyl)-4- (cycloheptylmethyl)-2-methyl-2,4-dihydro- 3H-1,2,4-triazol-3-one |
| 13 | 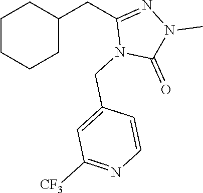 [SMILES: Cn1nc(CC2CCCCC2)n(Cc2ccnc(C(F)(F)F)c2)c1=O] | 5-(cyclohexylmethyl)-2-methyl-4-((2- (trifluoromethyl)pyridin-4-yl)methyl)-2,4- dihydro-3H-1,2,4-triazol-3-one |
| 14 | 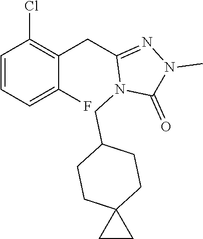 [SMILES: Cn1nc(Cc2c(F)cccc2Cl)n(CC2CCC3(CC2)CC3)c1=O] | 5-(2-chloro-6-fluorobenzyl)-2-methyl-4- (spiro[2.5]octan-6-ylmethyl)-2,4-dihydro- 3H-1,2,4-triazol-3-one |
| 15 | 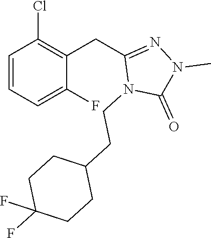 [SMILES: Cn1nc(Cc2c(F)cccc2Cl)n(CCC2CCC(F)(F)CC2)c1=O] | 5-(2-chloro-6-fluorobenzyl)-4-(2-(4,4- difluorocyclohexyl)ethyl)-2-methyl-2,4- dihydro-3H-1,2,4-triazol-3-one |
| 16 | 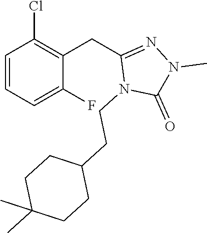 | 5-(2-chloro-6-fluorobenzyl)-4-(2-(4,4- dimethylcyclohexyl)ethyl)-2-methyl-2,4- dihydro-3H-1,2,4-triazol-3-one |
| 17 | 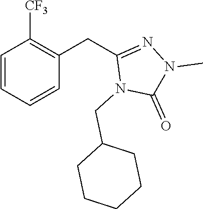 [SMILES: Cn1nc(Cc2ccccc2C(F)(F)F)n(CC2CCCCC2)c1=O] | 4-(cyclohexylmethyl)-2-methyl-5-(2- (trifluoromethyl)benzyl)-2,4-dihydro-3H- 1,2,4-triazol-3-one |
| 18 | 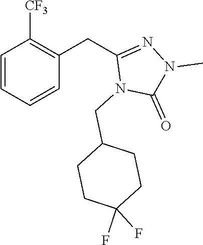 [SMILES: Cn1nc(Cc2ccccc2C(F)(F)F)n(CC2CCC(F)(F)CC2)c1=O] | 4-((4,4-difluorocyclohexyl)methyl)-2- methyl-5-(2-(trifluoromethyl)benzyl)-2,4- dihydro-3H-1,2,4-triazol-3-one |
| 19 | 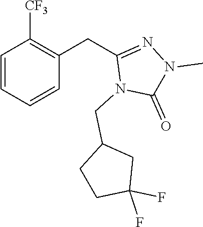 [SMILES: Cn1nc(Cc2ccccc2C(F)(F)F)n(CC2CCC(F)(F)C2)c1=O] | 4-((3,3-difluorocyclopentyl)methyl)-2- methyl-5-(2-(trifluoromethyl)benzyl)-2,4- dihydro-3H-1,2,4-triazol-3-one |
| 20 | 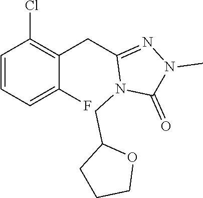 [SMILES: Cn1nc(Cc2c(F)cccc2Cl)n(CC2CCCO2)c1=O] | 5-(2-chloro-6-fluorobenzyl)-2-methyl-4- ((tetrahydrofuran-2-yl)methyl)-2,4-dihydro- 3H-1,2,4-triazol-3-one |
| 21 | 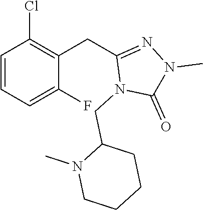 [SMILES: CN1CCCCC1Cn1c(Cc2c(F)cccc2Cl)nn(C)c1=O] | 5-(2-chloro-6-fluorobenzyl)-2-methyl-4-((1- methylpiperidin-2-yl)methyl)-2,4-dihydro- 3H-1,2,4-triazol-3-one |
| 22 | 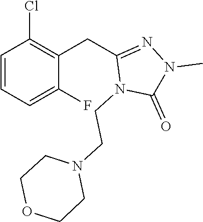 [SMILES: Cn1nc(Cc2c(F)cccc2Cl)n(CCN2CCOCC2)c1=O] | 5-(2-chloro-6-fluorobenzyl)-2-methyl-4-(2- morpholinoethyl)-2,4-dihydro-3H-1,2,4- triazol-3-one |
| 23 | 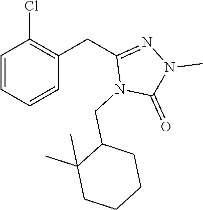 [SMILES: Cn1nc(Cc2ccccc2Cl)n(CC2CCCCC2(C)C)c1=O] | 4-((2,2-dimethylcyclohexyl)methyl)-2- methyl-5-(2-(trifluoromethyl)benzyl)-2,4- dihydro-3H-1,2,4-triazol-3-one |
| 24 | 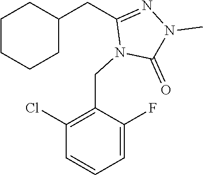 [SMILES: Cn1nc(CC2CCCCC2)n(Cc2c(F)cccc2Cl)c1=O] | 4-(2-chloro-6-fluorobenzyl)-5- (cyclohexylmethyl)-2-methyl-2,4-dihydro- 3H-1,2,4-triazol-3-one |
| 25 | 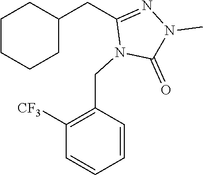 [SMILES: Cn1nc(CC2CCCCC2)n(Cc2ccccc2C(F)(F)F)c1=O] | 5-(cyclohexylmethyl)-2-methyl-4-(2- (trifluoromethyl)benzyl)-2,4-dihydro-3H- 1,2,4-triazol-3-one |
| 26 | 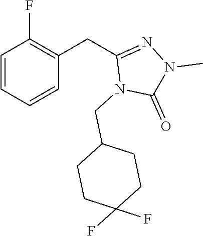 [SMILES: Cn1nc(Cc2ccccc2F)n(CC2CCC(F)(F)CC2)c1=O] | 4-((4,4-difluorocyclohexyl)methyl)-5-(2- fluorobenzyl)-2-methyl-2,4-dihydro-3H- 1,2,4-triazol-3-one |
| 27 | 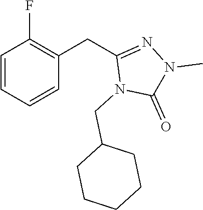 [SMILES: Cn1nc(Cc2ccccc2F)n(CC2CCCCC2)c1=O] | 4-(cyclohexylmethyl)-5-(2-fluorobenzyl)-2- methyl-2,4-dihydro-3H-1,2,4-triazol-3-one |
| 28 | 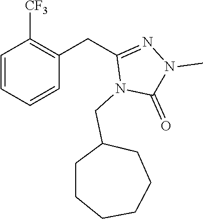 [SMILES: Cn1nc(Cc2ccccc2C(F)(F)F)n(CC2CCCCCC2)c1=O] | 4-(cycloheptylmethyl)-2-methyl-5-(2- (trifluoromethyl)benzyl)-2,4-dihydro-3H- 1,2,4-triazol-3-one |
| 29 | 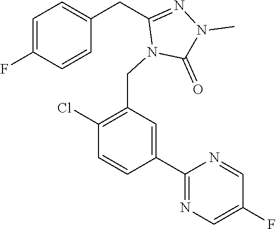 [SMILES: Cn1nc(Cc2ccc(F)cc2)n(Cc2cc(-c3ncc(F)cn3)ccc2Cl)c1=O] | 4-(2-chloro-5-(5-fluoropyrimidin-2- yl)benzyl)-5-(4-fluorobenzyl)-2-methyl-2,4- dihydro-3H-1,2,4-triazol-3-one |
| 30 | 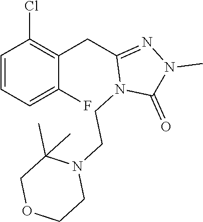 [SMILES: Cn1nc(Cc2c(F)cccc2Cl)n(CCN2CCOCC2(C)C)c1=O] | 5-(2-chloro-6-fluorobenzyl)-4-(2-(3,3- dimethylmorpholino)ethyl)-2-methyl-2,4- dihydro-3H-1,2,4-triazol-3-one |
| 31 | 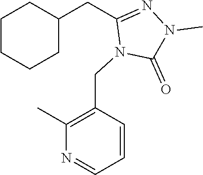 [SMILES: Cc1ncccc1Cn1c(CC2CCCCC2)nn(C)c1=O] | 5-(cyclohexylmethyl)-2-methyl-4-((2- methylpyridin-3-yl)methyl)-2,4-dihydro- 3H-1,2,4-triazol-3-one |
| 32 | 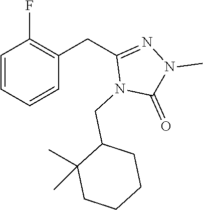 [SMILES: Cn1nc(Cc2ccccc2F)n(CC2CCCCC2(C)C)c1=O] | 4-((2,2-dimethylcyclohexyl)methyl)-5-(2- fluorobenzyl)-2-methyl-2,4-dihydro-3H- 1,2,4-triazol-3-one |
| 33 | 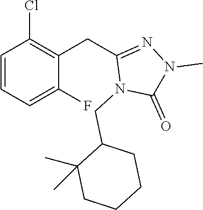 [SMILES: Cn1nc(Cc2c(F)cccc2Cl)n(CC2CCCCC2(C)C)c1=O] | 5-(2-chloro-6-fluorobenzyl)-4-((2,2- dimethylcyclohexyl)methyl)-2-methyl-2,4- dihydro-3H-1,2,4-triazol-3-one |
| 34 | 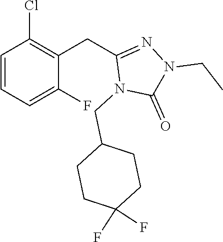 [SMILES: CCn1nc(Cc2c(F)cccc2Cl)n(CC2CCC(F)(F)CC2)c1=O] | 5-(2-chloro-6-fluorobenzyl)-4-((4,4- difluorocyclohexyl)methyl)-2-ethyl-2,4- dihydro-3H-1,2,4-triazol-3-one |
| 35 | 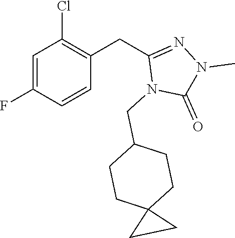 [SMILES: Cn1nc(Cc2ccc(F)cc2Cl)n(CC2CCC3(CC2)CC3)c1=O] | 5-(2-chloro-4-fluorobenzyl)-2-methyl-4- (spiro[2.5]octan-6-ylmethyl)-2,4-dihydro- 3H-1,2,4-triazol-3-one |
| 36 | 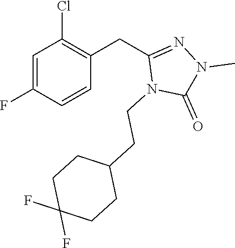 [SMILES: Cn1nc(Cc2ccc(F)cc2Cl)n(CCC2CCC(F)(F)CC2)c1=O] | 5-(2-chloro-4-fluorobenzyl)-4-(2-(4,4- difluorocyclohexyl)ethyl)-2-methyl-2,4- dihydro-3H-1,2,4-triazol-3-one |
| 37 | 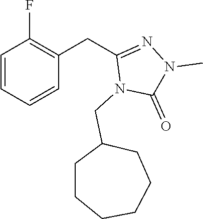 [SMILES: Cn1nc(Cc2ccccc2F)n(CC2CCCCCC2)c1=O] | 4-(cycloheptylmethyl)-5-(2-fluorobenzyl)-2- methyl-2,4-dihydro-3H-1,2,4-triazol-3-one |
| 38 | 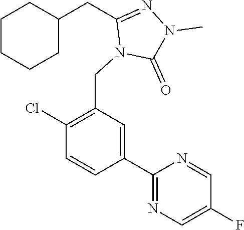 [SMILES: Cn1nc(CC2CCCCC2)n(Cc2cc(-c3ncc(F)cn3)ccc2Cl)c1=O] | 4-(2-chloro-5-(5-fluoropyrimidin-2- yl)benzyl)-5-(cyclohexylmethyl)-2-methyl- 2,4-dihydro-3H-1,2,4-triazol-3-one |
| 39 | 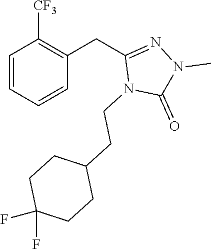 [SMILES: Cn1nc(Cc2ccccc2C(F)(F)F)n(CCC2CCC(F)(F)CC2)c1=O] | 4-(2-(4,4-difluorocyclohexyl)ethyl)-2- methyl-5-(2-(trifluoromethyl)benzyl)-2,4- dihydro-3H-1,2,4-triazol-3-one |
| 40 | 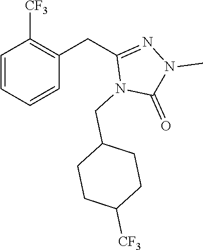 [SMILES: Cn1nc(Cc2ccccc2C(F)(F)F)n(CC2CCC(C(F)(F)F)CC2)c1=O] | 2-methyl-5-(2-(trifluoromethyl)benzyl)-4- ((4-(trifluoromethyl)cyclohexyl)methyl)- 2,4-dihydro-3H-1,2,4-triazol-3-one |
| 41 | 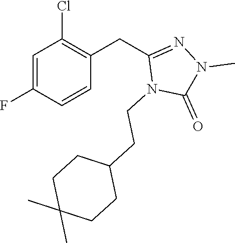 [SMILES: Cn1nc(Cc2ccc(F)cc2Cl)n(CCC2CCC(C)(C)CC2)c1=O] | 5-(2-chloro-4-fluorobenzyl)-4-(2-(4,4- dimethylcyclohexyl)ethyl)-2-methyl-2,4- dihydro-3H-1,2,4-triazol-3-one |
| 42 | 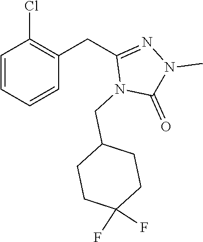 [SMILES: Cn1nc(Cc2ccccc2Cl)n(CC2CCC(F)(F)CC2)c1=O] | 5-(2-chlorobenzyl)-4-((4,4- difluorocyclohexyl)methyl)-2-methyl-2,4- dihydro-3H-1,2,4-triazol-3-one |
| 43 | 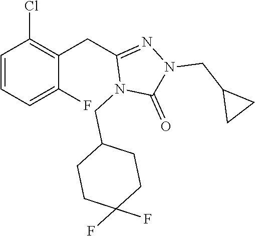 [SMILES: O=c1n(CC2CC2)nc(Cc2c(F)cccc2Cl)n1CC1CCC(F)(F)CC1] | 5-(2-chloro-6-fluorobenzyl)-2- (cyclopropylmethyl)-4-((4,4- difluorocyclohexyl)methyl)-2,4-dihydro- 3H-1,2,4-triazol-3-one |
| 44 | 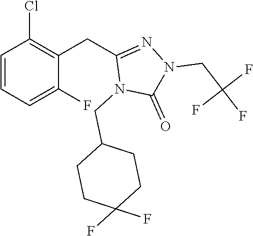 [SMILES: O=c1n(CC(F)(F)F)nc(Cc2c(F)cccc2Cl)n1CC1CCC(F)(F)CC1] | 5-(2-chloro-6-fluorobenzyl)-4-((4,4- difluorocyclohexyl)methyl)-2-(2,2,2- trifluoroethyl)-2,4-dihydro-3H-1,2,4-triazol- 3-one |
| 45 | 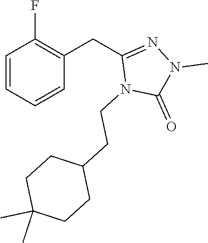 | 4-(2-(4,4-dimethylcyclohexyl)ethyl)-5-(2- fluorobenzyl)-2-methyl-2,4-dihydro-3H- 1,2,4-triazol-3-one |
| 46 | 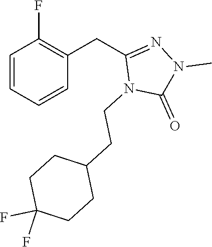 [SMILES: Cn1nc(Cc2ccccc2F)n(CCC2CCC(F)(F)CC2)c1=O] | 4-(2-(4,4-difluorocyclohexyl)ethyl)-5-(2- fluorobenzyl)-2-methyl-2,4-dihydro-3H- 1,2,4-triazol-3-one |
| 47 | 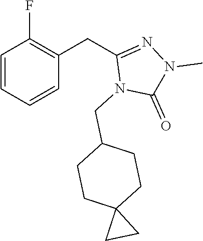 [SMILES: Cn1nc(Cc2ccccc2F)n(CC2CCC3(CC2)CC3)c1=O] | 5-(2-fluorobenzyl)-2-methyl-4- (spiro[2.5]octan-6-ylmethyl)-2,4-dihydro- 3H-1,2,4-triazol-3-one |
| 48 | 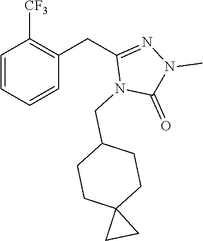 [SMILES: Cn1nc(Cc2ccccc2C(F)(F)F)n(CC2CCC3(CC2)CC3)c1=O] | 2-methyl-4-(spiro[2.5]octan-6-ylmethyl)-5- (2-(trifluoromethyl)benzyl)-2,4-dihydro-3H- 1,2,4-triazol-3-one |
| 49 | 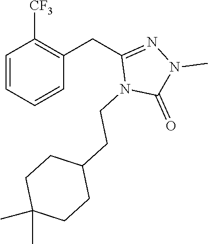 | 4-(2-(4,4-dimethylcyclohexyl)ethyl)-2- methyl-5-(2-(trifluoromethyl)benzyl)-2,4- dihydro-3H-1,2,4-triazol-3-one |
| 50 | 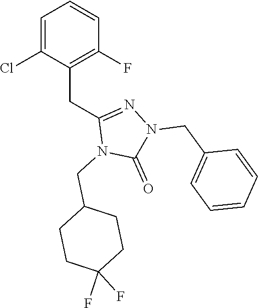 [SMILES: O=c1n(Cc2ccccc2)nc(Cc2c(F)cccc2Cl)n1CC1CCC(F)(F)CC1] | 2-benzyl-5-(2-chloro-6-fluorobenzyl)-4- ((4,4-difluorocyclohexyl)methyl)-2,4- dihydro-3H-1,2,4-triazol-3-one |
| 51 | 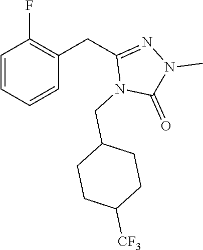 [SMILES: Cn1nc(Cc2ccccc2F)n(CC2CCC(C(F)(F)F)CC2)c1=O] | 5-(2-fluorobenzyl)-2-methyl-4-((4- (trifluoromethyl)cyclohexyl)methyl)-2,4- dihydro-3H-1,2,4-triazol-3-one |
| 52 | 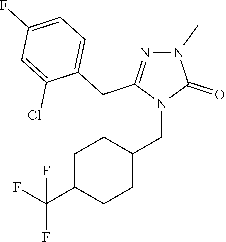 [SMILES: Cn1nc(Cc2ccc(F)cc2Cl)n(CC2CCC(C(F)(F)F)CC2)c1=O] | 5-(2-chloro-4-fluorobenzyl)-2-methyl-4-((4- (trifluoromethyl)cyclohexyl)methyl)-2,4- dihydro-3H-1,2,4-triazol-3-one |
| 53 | 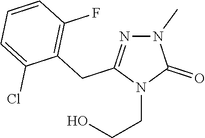 [SMILES: Cn1nc(Cc2c(F)cccc2Cl)n(CCO)c1=O] | 5-(2-chloro-6-fluorobenzyl)-4-(2- hydroxyethyl)-2-methyl-2,4- dihydro-3H-1,2,4-triazol-3-one |
| 54 | 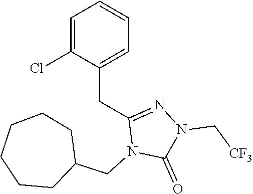 [SMILES: O=c1n(CC(F)(F)F)nc(Cc2ccccc2Cl)n1CC1CCCCCC1] | 5-[(2-chlorophenyl)methyl]-4- (cycloheptylmethyl)-2-(2,2,2- trifluoroethyl)-2,4-dihydro-3H-1,2,4-triazol- 3-one |
| 55 | 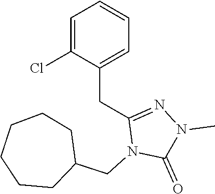 [SMILES: Cn1nc(Cc2ccccc2Cl)n(CC2CCCCCC2)c1=O] | 5-[(2-chlorophenyl)methyl]-4- (cycloheptylmethyl)-2-methyl-2,4-dihydro- 3H-1,2,4-triazol-3-one |
| 56 | 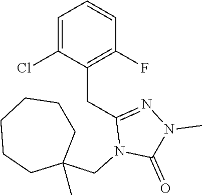 [SMILES: Cn1nc(Cc2c(F)cccc2Cl)n(CC2(C)CCCCCC2)c1=O] | 5-[(2-chloro-6-fluorophenyl)methyl]-2- methyl-4-[(1-methylcycloheptyl)methyl]- 2,4-dihydro-3H-1,2,4-triazol-3-one |
| 57 | 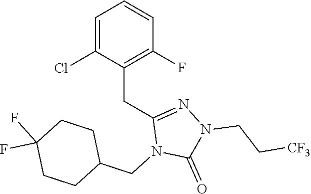 [SMILES: O=c1n(CCC(F)(F)F)nc(Cc2c(F)cccc2Cl)n1CC1CCC(F)(F)CC1] | 5-[(2-chloro-6-fluorophenyl)methyl]-4- [(4,4-difluorocyclohexyl)methyl]-2-(3,3,3- trifluoropropyl)-2,4-dihydro-3H-1,2,4- triazol-3-one |
| 58 | 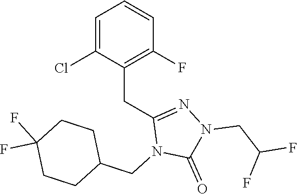 [SMILES: O=c1n(CC(F)F)nc(Cc2c(F)cccc2Cl)n1CC1CCC(F)(F)CC1] | 5-[(2-chloro-6-fluorophenyl)methyl]-4- [(4,4-difluorocyclohexyl)methyl]-2-(2,2- difluoroethyl)-2,4-dihydro-3H-1,2,4-triazol- 3-one |
| 59 | 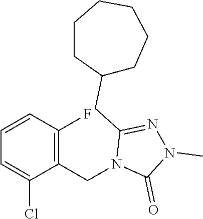 [SMILES: Cn1nc(CC2CCCCCC2)n(Cc2c(F)cccc2Cl)c1=O] | 4-[(2-chloro-6-fluorophenyl)methyl]-5- (cycloheptylmethyl)-2-methyl-2,4-dihydro- 3H-1,2,4-triazol-3-one |
| 60 | 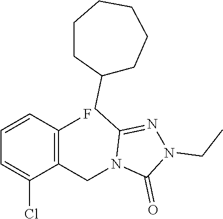 [SMILES: CCn1nc(CC2CCCCCC2)n(Cc2c(F)cccc2Cl)c1=O] | 4-[(2-chloro-6-fluorophenyl)methyl]-5- (cycloheptylmethyl)-2-ethyl-2,4-dihydro- 3H-1,2,4-triazol-3-one |
| 61 | 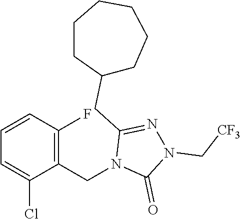 [SMILES: O=c1n(CC(F)(F)F)nc(CC2CCCCCC2)n1Cc1c(F)cccc1Cl] | 4-[(2-chloro-6-fluorophenyl)methyl]-5- (cycloheptylmethyl)-2-(2,2,2- trifluoroethyl)-2,4-dihydro-3H-1,2,4-triazol- 3-one |
| 62 | 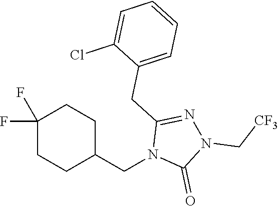 [SMILES: O=c1n(CC(F)(F)F)nc(Cc2ccccc2Cl)n1CC1CCC(F)(F)CC1] | 5-[(2-chlorophenyl)methyl]-4-[(4,4- difluorocyclohexyl)methyl]-2-(2,2,2- trifluoroethyl)-2,4-dihydro-3H-1,2,4-triazol- 3-one |
| 63 | 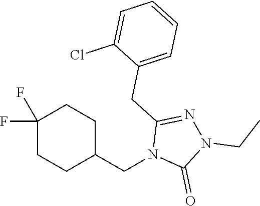 [SMILES: CCn1nc(Cc2ccccc2Cl)n(CC2CCC(F)(F)CC2)c1=O] | 5-[(2-chlorophenyl)methyl]-4-[(4,4- difluorocyclohexyl)methyl]-2-ethyl-2,4- dihydro-3H-1,2,4-triazol-3-one |
| 64 | 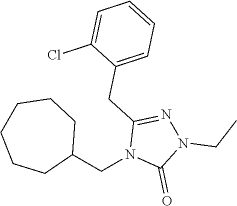 [SMILES: CCn1nc(Cc2ccccc2Cl)n(CC2CCCCCC2)c1=O] | 5-[(2-chlorophenyl)methyl]-4- (cycloheptylmethyl)-2-ethyl-2,4-dihydro- 3H-1,2,4-triazol-3-one |
| 65 | 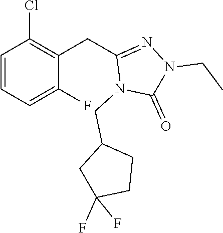 [SMILES: CCn1nc(Cc2c(F)cccc2Cl)n(CC2CCC(F)(F)C2)c1=O] | 5-[(2-chloro-6-fluorophenyl)methyl]-4- [(3,3-difluorocyclopentyl)methyl]-2-ethyl- 2,4-dihydro-3H-1,2,4-triazol-3-one |
| 66 | 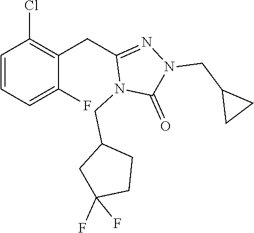 [SMILES: O=c1n(CC2CC2)nc(Cc2c(F)cccc2Cl)n1CC1CCC(F)(F)C1] | 5-[(2-chloro-6-fluorophenyl)methyl]-2- (cyclopropylmethyl)-4-[(3,3- difluorocyclopentyl)methyl]-2,4-dihydro- 3H-1,2,4-triazol-3-one |
| 67 | 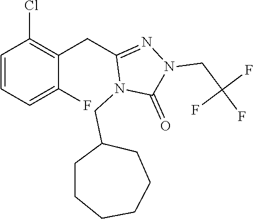 [SMILES: O=c1n(CC(F)(F)F)nc(Cc2c(F)cccc2Cl)n1CC1CCCCCC1] | 5-[(2-chloro-6-fluorophenyl)methyl]-4- (cycloheptylmethyl)-2-(2,2,2- trifluoroethyl)-2,4-dihydro-3H-1,2,4-triazol- 3-one |
| 68 | 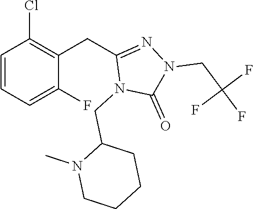 [SMILES: CN1CCCCC1Cn1c(Cc2c(F)cccc2Cl)nn(CC(F)(F)F)c1=O] | 5-[(2-chloro-6-fluorophenyl)methyl]-4-[(1- methylpiperidin-2-yl)methyl]-2-(2,2,2- trifluoroethyl)-2,4-dihydro-3H-1,2,4-triazol- 3-one |
| 69 | 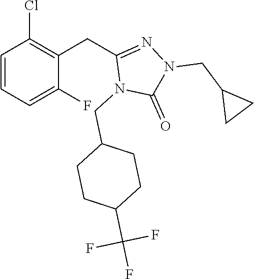 [SMILES: O=c1n(CC2CC2)nc(Cc2c(F)cccc2Cl)n1CC1CCC(C(F)(F)F)CC1] | 5-(2-chloro-6-fluorobenzyl)-2- (cyclopropylmethyl)-4-((4- (trifluoromethyl)cyclohexyl)methyl)-2,4- dihydro-3H-1,2,4-triazol-3-one |
| 70 | 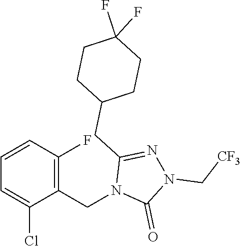 [SMILES: O=c1n(CC(F)(F)F)nc(CC2CCC(F)(F)CC2)n1Cc1c(F)cccc1Cl] | 4-[(2-chloro-6-fluorophenyl)methyl]-5- [(4,4-difluorocyclohexyl)methyl]-2-(2,2,2- trifluoroethyl)-2,4-dihydro-3H-1,2,4-triazol- 3-one |
| 71 | 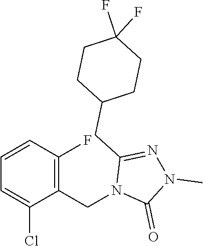 [SMILES: Cn1nc(CC2CCC(F)(F)CC2)n(Cc2c(F)cccc2Cl)c1=O] | 4-[(2-chloro-6-fluorophenyl)methyl]-5- [(4,4-difluorocyclohexyl)methyl]-2-methyl- 2,4-dihydro-3H-1,2,4-triazol-3-one |

### Analytical Part

### System Purification

### HPLC Preparative

HPLC system WATERS Quaternary Gradient Mobile 2535 equipped with WATERS UV/Visible Detector 2489 set to a dual-wavelength UV detection. Two mobile phases were used, mobile phase A: water (MilliQ) 0.05% FA; mobile phase B: acetonitrile (Chromasolv Sigma-Aldrich) 0.05% FA, and the run gradient conditions were set specifically for each compound. The purifications were achieved on a Luna Phenomenex Column C18 5 μm 19×150. An injection volume between 100 and 500 μl was used and the flow was 15 ml/min.

### LCMS—Procedure 1

The HPLC measurement was performed using a Dionex 3000 module comprising a quaternary pump with degasser, an autosampler, a column oven (set at 29° C.), a diode-array detector DAD and a column as specified in the respective methods below. Flow from the column was split to a MS spectrometer. The MS detector (LCQ Fleet Thermo Scientific) was configured with an electrospray ionization source. Mass spectra were acquired by scanning from 50 to 800 in 0.48 second. The capillary needle voltage was 5 kV in positive and negative ionization mode and the source temperature was maintained at 275° C. Nitrogen was used as the nebulizer gas, the flow was 8 l/min. Data acquisition was performed with Thermo Xcalibur Qual Browser.

In addition to general procedure: Reversed phase HPLC was carried out on a Kinetex XB-C18 column Phenomenex (1.7 μm, 50×2.1 mm) with a flow rate of 0.300 ml/min. Two mobile phases were used, mobile phase A: ammonium formate buffer solution at pH 3.5; mobile phase B: acetonitrile (Chromasolv Sigma-Aldrich), and they were employed to run a gradient conditions from 15% B to 50% in 15 minutes, 100% B in 0.9 minutes and 5% B in 0.1 minutes and hold these conditions for 4 minutes in order to reequilibrate the column. An injection volume of 5 μl was used.

### LCMS Procedure 2

HPLC measurement was performed using a VANQUISH FLEX module comprising a quaternary pump with degasser, an autosampler, a column oven (set at 40° C.), a diode-array detector DAD and a column as specified in the respective methods below. The MS detector (ISQ Thermo Scientific) was configured with an electrospray ionization source.

Mass spectra were acquired by scanning from 100 to 700 in 0.2 second. The capillary needle voltage was 3 kV in positive and 2 kV in negative ionization mode and the source temperature was maintained at 250° C. Nitrogen was used as the nebulizer gas.

In addition to general procedure: Reversed phase UHPLC was carried out on a Luna Omega-C18 column Phenomenex (1.6 μm, 50×2.1 mm) with a flow rate of 0.600 ml/min. Two mobile phases were used, mobile phase A: water (LC-MS grade) 0.1% FA; mobile phase B: acetonitrile (LiChrosolv for LC-MS Merck), and they were employed to run a gradient conditions from 15% B for 0.2 minutes, from 15% to 95% in 1.6 minutes, 95% B for 0.60 minutes and 15% B in 0.10 minutes and hold these conditions for 1.05 minutes in order to reequilibrate the column (Total Run Time 3.55 minutes). An injection volume of 0.8 μl was used. Data acquisition was performed with Chromeleon 7

TABLE 2
Retention time (Rₜ) in minutes, [M + H]⁺, LCMS procedure

| | | [M + | Proce- | | | [M + | Proce- |
|---|---|---|---|---|---|---|---|
| Example | Rₜ | H]⁺ | dure | Example | Rₜ | H]⁺ | dure |
| 1 | 4.9 | 350.2 | 1 | 2 | 5.3 | 338.3 | 1 |
| 3 | 5.0 | 374.2 | 1 | 4 | 4.9 | 360.5 | 1 |
| 5 | 5.0 | 372.1 | 1 | 6 | 5.0 | 356.0 | 1 |
| 7 | 5.4 | 406.4 | 1 | 8 | 5.0 | 349.9 | 1 |
| 9 | 5.5 | 338.3 | 1 | 10 | 5.0 | 359.9 | 1 |
| 11 | 5.7 | 352.0 | 1 | 12 | 5.6 | 352.1 | 1 |
| 13 | 2.4 | 355.3 | 2 | 14 | 2.7 | 364.0 | 2 |
| 15 | 2.5 | 388.3 | 2 | 16 | 2.9 | 380.3 | 2 |
| 17 | 2.3 | 354.3 | 2 | 18 | 2.1 | 390.0 | 2 |
| 19 | 2.1 | 376.0 | 2 | 20 | 2.2 | 326.3 | 2 |
| 21 | 1.6 | 353.1 | 2 | 22 | 1.6 | 355.3 | 2 |
| 23 | 2.8 | 382.3 | 2 | 24 | 2.6 | 338.1 | 2 |
| 25 | 2.7 | 354.1 | 2 | 26 | 2.4 | 340.1 | 2 |
| 27 | 2.5 | 304.3 | 2 | 28 | 2.7 | 368.1 | 2 |
| 29 | 2.5 | 428.1 | 2 | 30 | 1.8 | 383.2 | 2 |
| 31 | 1.7 | 301.2 | 2 | 32 | 2.6 | 332.3 | 2 |
| 33 | 2.7 | 366.1 | 2 | 34 | 2.7 | 388.2 | 2 |
| 35 | 2.7 | 364.1 | 2 | 36 | 2.5 | 388.2 | 2 |
| 37 | 2.6 | 318.1 | 2 | 38 | 2.7 | 416.4 | 2 |
| 39 | 2.5 | 404.3 | 2 | 40 | 2.6 | 422.0 | 2 |
| 41 | 2.9 | 380.4 | 2 | 42 | 2.4 | 356.4 | 2 |
| 43 | 2.6 | 414.4 | 2 | 44 | 2.6 | 442.3 | 2 |
| 45 | 2.8 | 346.1 | 2 | 46 | 2.4 | 354.3 | 2 |
| 47 | 2.6 | 330.3 | 2 | 48 | 2.7 | 380.2 | 2 |
| 49 | 2.9 | 396.1 | 2 | 50 | 2.7 | 450.3 | 2 |
| 51 | 2.5 | 372.2 | 2 | 52 | 2.6 | 406.2 | 2 |
| 53 | 1.8 | 286.14 | 2 | 54 | 2.8 | 402.3 | 2 |
| 55 | 2.7 | 334.2 | 2 | 56 | 2.7 | 366.2 | 2 |
| 57 | 2.5 | 456.1 | 2 | 58 | 2.5 | 424.1 | 2 |
| 59 | 2.7 | 352.2 | 2 | 60 | 2.7 | 366.2 | 2 |
| 61 | 2.8 | 420.2 | 2 | 62 | 2.6 | 465.2* | 2 |
| 63 | 2.5 | 370.3 | 2 | 64 | 2.7 | 348.2 | 2 |
| 65 | 2.5 | 374.2 | 2 | 66 | 2.40 | 400.2 | 2 |
| 67 | 2.8 | 420.2 | 2 | 68 | 1.9 | 421.1 | 2 |
| 69 | 2.8 | 446.3 | 2 | 70 | 2.6 | 442.8 | 2 |
| 71 | 2.5 | 374.3 | 2 | | | | |

*Acetonitrile adduct

### NMR Characterization

¹H NMVR spectra were recorded on a Bruker NMVR 400 MHz or 600 MHz spectrometers using CDCl₃, as solvent. Chemical shifts (δ) are reported in parts per million (ppm) relative to tetramethylsilane (TMS), wich was used as internal standard.

| AXXAM CODE | ¹H-NMR |
|---|---|
| 1 | ¹H NMR (CDCl₃) δ ppm 3.36-3.46 (s, 3 H) 3.82-3.92 (s, 2 H) |
| | 4.88 (s, 2 H) 6.93-7.06 (m, 3 H) 7.13-7.24 (m, 4 H) |
| 2 | ¹H NMR (CDCl₃) δ ppm 0.98 (m, 2 H) 1.09-1.27 (m, 4 H) 1.57- |
| | 1.79 (m, 5 H) 3.34 (s, 3 H) 3.47 (d, J = 7.24 Hz, 2 H) 3.97 (s, 2 |
| | H) 6.97-7.09 (m, 1 H) 7.18-7.26 (m, 2 H) |
| 3 | ¹H NMR (CDCl₃) δ ppm 1.26-1.43 (m, 2 H) 1.54-1.68 (m, 1 |
| | H) 1.68-1.79 (m, 3 H) 1.79-1.91 (m, 1 H) 2.03-2.17 (m, 2 H) |
| | 3.34 (s, 3 H) 3.53 (d, J = 7.42 Hz, 2 H) 3.97 (s, 2 H) 7.00-7.07 |
| | (m, 1 H) 7.17-7.27 (m, 2 H) |
| 4 | ¹H NMR (CDCl₃) δ ppm 1.58 (dq, J = 12.83, 9.01 Hz, 1 H) 1.77- |
| | 1.96 (m, 2 H) 1.96-2.12 (m, 1 H) 2.12-2.31 (m, 2 H) 2.52 (m, |
| | 1 H) 3.33 (s, 3 H) 3.65 (d, J = 7.61 Hz, 2 H) 3.97 (s, 2 H) 6.98- |
| | 7.07 (m, 1 H) 7.19-7.28 (m, 2 H) |
| 5 | ¹H NMR (CDCl₃) δ ppm 1.75-1.87 (m, 2 H) 1.91-1.98 (m, 2 |
| | H) 2.04 (br dd, J = 13.15, 7.56 Hz, 2 H) 2.16-2.32 (m, 1 H) 3.33 |
| | (s, 3 H) 3.60 (d, J = 7.42 Hz, 2 H) 3.96 (s, 2 H) 6.96-7.07 (m, 1 |
| | H) 7.17-7.31 (m, 2 H) |
| 6 | ¹H NMR (CDCl₃) δ ppm 1.02-1.15 (m, 1 H) 1.35-1.61 (m, 4 |
| | H) 1.73-1.93 (m, 2 H) 1.99-2.17 (m, 2 H) 3.35 (br s, 3 H) 3.51 |
| | (br s, 2 H) 3.94-4.05 (m, 1 H) 7.04 (ddd, J = 9.58, 6.97, 2.34 Hz, |
| | 1 H) 7.19-7.32 (m, 2 H) |
| 7 | ¹H NMR (CDCl₃) δ ppm 0.99-1.11 (m, 1 H) 1.17-1.31 (m, 2 |
| | H) 1.48-1.58 (m, 2 H) 1.63-1.84 (m, 4 H) 1.90-1.99 (m, 1 H) |
| | 3.35 (d, J = 3.12 Hz, 3 H) 3.50 (d, J = 7.42 Hz, 1 H) 3.62 (d, J = 7.79 |
| | Hz, 1 H) 3.97 (s, 2 H) 6.95-7.09 (m, 1 H) 7.16-7.31 (m, 2 H) |
| 8 | ¹H NMR (CDCl₃) δ ppm 3.46 (s, 3 H) 3.80 (s, 2 H) 4.66 (s, 2 H) |
| | 6.86 (td, J = 8.25, 2.57 Hz, 1 H) 6.93 (t, J = 8.61 Hz, 2 H) 7.00 (dd, |
| | J = 8.52, 5.96 Hz, 1 H) 7.06-7.15 (m, 3 H) |
| 9 | ¹H NMR (CDCl₃) δ ppm 0.82-0.98 (m, 2 H) 1.11 (br s, 2 H) 1.50- |
| | 1.65 (m, 5 H) 1.65-1.76 (m, 2 H) 3.29 (d, J = 7.15 Hz, 2 H) 3.41 |
| | (s, 3 H) 3.92 (s, 2 H) 6.94 (td, J = 8.25, 2.57 Hz, 1 H) 7.09-7.20 |
| | (m, 2 H) |
| 10 | ¹H NMR (CDCl₃) δ ppm 1.49 (dq, J = 12.96, 9.03 Hz, 1 H) 1.68- |
| | 1.92 (m, 2 H) 1.92-2.08 (m, 1 H) 2.08-2.24 (m, 2 H) 2.34-2.46 |
| | (m, 1 H) 3.37-3.45 (s, 3 H) 3.49 (d, J = 7.61 Hz, 2 H) 3.94 (s, 2 |
| | H) 6.96 (td, J = 8.20, 2.66 Hz, 1 H) 7.14-7.23 (m, 2 H) |
| 11 | ¹H NMR (CDCl₃) δ ppm 1.05-1.17 (m, 2 H) 1.30 (dt, J = 12.60, |
| | 9.51 Hz, 2 H) 1.38-1.68 (m, 8 H) 1.71-1.82 (m, 1 H) 3.29 (d, |
| | J = 7.79 Hz, 2 H) 3.41 (s, 3 H) 3.93 (s, 2 H) 6.95 (td, J = 8.27, 2.52 |
| | Hz, 1 H) 7.12-7.20 (m, 2 H) |
| 12 | ¹H NMR (CDCl₃) δ ppm 1.11-1.27 (m, 2 H) 1.31-1.45 (m, 2 |
| | H) 1.45-1.62 (m, 4 H) 1.61-1.74 (m, 4 H) 1.86-1.99 (m, 1 H) |
| | 3.42 (s, 3 H) 3.46 (d, J = 7.79 Hz, 2 H) 3.97 (s, 2 H) 6.98-7.08 |
| | (m, 1 H) 7.19-7.27 (m, 2 H) |
| 13 | ¹H NMR (CDCl₃) δ ppm 0.79-0.91 (m, 2 H) 1.03-1.10 (m, 3 |
| | H) 1.38-1.48 (m, 1 H) 1.54-1.65 (m, 5 H) 2.19 (d, J = 7.08 Hz, |
| | 2 H) 3.42 (s, 3 H) 4.82 (s, 2 H) 7.25 (d, J = 4.72 Hz, 1 H) 7.44 (s, |
| | 1 H) 8.64 (d, J = 5.09 Hz, 1 H) |
| 14 | ¹H NMR (CDCl₃) δ ppm −0.07-0.04 (m, 2 H) 0.04-0.16 (m, 2 |
| | H) 0.70 (br d, J = 13.63 Hz, 2 H) 0.93-1.12 (m, 2 H) 1.42-1.66 |
| | (m, 5 H) 3.17 (s, 3 H) 3.35 (d, J = 7.63 Hz, 2 H) 3.81 (s, 2 H) 6.79- |
| | 6.91 (m, 1 H) 7.02-7.13 (m, 2 H) |
| 15 | ¹H NMR (CDCl₃) δ ppm 1.14-1.29 (m, 2 H) 1.29-1.41 (m, 1 |
| | H) 1.42-1.52 (m, 2 H) 1.54-1.83 (br s, 4 H) 1.96-2.08 (m, 2 |
| | H) 3.25 (s, 3 H) 3.58-3.64 (m, 2 H) 3.93 (s, 2 H) 6.93-7.04 (m, |
| | 1 H) 7.14-7.27 (m, 2 H) |
| 16 | ¹H NMR (CDCl₃) δ ppm 0.79 (s, 3 H) 0.82 (s, 3 H) 0.98-1.20 |
| | (m, 5 H) 1.26-1.34 (m, 2 H) 1.40-1.56 (m, 4 H) 3.28 (s, 3 H) |
| | 3.55-3.66 (m, 2 H) 3.93 (s, 2 H) 6.93-7.05 (m, 1 H) 7.12-7.25 |
| | (m, 2 H) |
| 17 | ¹H NMR (CDCl₃) δ ppm 0.75-0.86 (m, 2 H) 0.93-1.08 (m, 3 |
| | H) 1.41-1.53 (br s, 4H) 1.58-1.65 (m, 2 H) 3.18 (d, J = 7.45 Hz, |
| | 2 H) 3.40 (s, 3 H) 4.00 (s, 2 H) 7.13 (d, J = 7.81 Hz, 1 H) 7.29- |
| | 7.41 (m, 1 H) 7.41-7.47 (m, 1 H) 7.64 (d, J = 7.81 Hz, 1 H) |
| 18 | ¹H NMR (CDCl₃) δ ppm 1.12-1.22 (m, 2 H) 1.38-1.56 (m, 5 |
| | H) 1.90-2.03 (m, 2 H) 3.25 (d, J = 7.08 Hz, 2 H) 3.40 (s, 3 H) |
| | 4.00 (s, 2 H) 7.16 (d, J = 7.63 Hz, 1 H) 7.31-7.40 (m, 1 H) 7.40- |
| | 7.50 (m, 1 H) 7.65 (d, J = 7.81 Hz, 1 H) |
| 19 | ¹H NMR (CDCl₃) δ ppm 1.40 (dq, J = 13.06, 9.09 Hz, 1 H) 1.62- |
| | 1.76 (m, 2 H) 1.82-1.98 (m, 1 H) 1.98-2.13 (m, 2 H) 2.17-2.29 |
| | (m, 1 H) 3.37 (d, J = 7.45 Hz, 2 H) 3.40 (s, 3 H) 4.00 (s, 2 H) 7.16 |
| | (d, J = 7.63 Hz, 1 H) 7.33-7.38 (m, 1 H) 7.43-7.47 (m, 1 H) 7.65 |
| | (d, J = 7.81 Hz, 1 H) |
| 20 | ¹H NMR (CDCl₃) δ ppm 1.31-1.40 (m, 1 H) 1.57-1.68 (m, 1 |
| | H) 1.77-1.85 (m, 1 H) 1.94-2.04 (m, 1 H) 3.22-3.33 (m, 3 H) |
| | 3.60-3.68 (m,2 H) 3.79-3.88 (m, 2 H) 4.06 (d, J = 1.25 Hz, 2 H) |
| | 4.07-4.13 (m, 1 H) 6.91-7.03 (m, 1 H) 7.13-7.21 (m, 2 H) |
| 21 | ¹H NMR (CDCl₃) δ ppm 1.08-1.32 (m, 2 H) 1.54 (br dd, J = 8.57, |
| | 4.06 Hz, 4 H) 1.66-1.76 (m, 1 H) 2.07-2.20 (m, 1 H) 2.37 (s, 3 |
| | H) 2.82 (br d, J = 11.76 Hz, 1 H) 3.28 (s, 3 H) 3.55 (br dd, J = 13.82, |
| | 9.32 Hz, 1 H) 3.89-3.97 (m, 1 H) 3.98-4.03 (m, 1 H) 6.94- |
| | 7.02 (m, 1 H) 7.15-7.21 (m, 3 H) |
| 22 | ¹H NMR (CDCl₃) δ ppm 2.41-2.50 (m, 4 H) 2.54 (t, J = 6.32 Hz, |
| | 2 H) 3.29 (s, 3 H) 3.61-3.67 (m, 4 H) 3.72 (t, J = 6.32 Hz, 2 H) |
| | 4.02 (d, J = 1.38 Hz, 2 H) 6.95-7.03 (m, 1 H) 7.16-7.22 (m, 2 H) |
| 23 | ¹H NMR (CDCl₃) δ ppm 0.72 (s, 3 H) 0.83 (s, 3 H) 1.00-1.13 |
| | (m, 2 H) 1.18-1.31 (m, 2 H) 1.34 (ddd, J = 11.73, 8.16, 3.63 Hz, |
| | 2 H) 1.48-1.66 (m, 3 H) 3.11-3.22 (m, 1 H) 3.39 (s, 3 H) 3.47- |
| | 3.55 (m, 1 H) 4.01 (br s, 2 H) 7.10 (d, J = 7.75 Hz, 1 H) 7.29- |
| | 7.39 (m, 1 H) 7.39-7.51 (m, 1 H) 7.64 (d, J = 7.88 Hz, 1 H) |
| 24 | ¹H NMR (CDCl₃) δ ppm 0.71-0.85 (m, 2 H) 0.99-1.13 (m, 2 |
| | H) 1.31 (dtt, J = 14.63, 7.30, 7.30, 3.77, 3.77 Hz, 1 H) 1.57 (br d, |
| | J = 9.38 Hz, 6 H) 2.17 (d, J = 7.25 Hz, 2 H) 3.31-3.40 (m, 3 H) |
| | 4.93 (s, 2 H) 6.90-6.98 (m, 1 H) 7.13-7.24 (br s, 2 H) |
| 25 | ¹H NMR (CDCl₃) δ ppm 0.85-0.97 (m, 2 H) 1.07-1.31 (m, 4 |
| | H) 1.53-1.74 (m, 5 H) 2.38 (d, J = 6.75 Hz, 2 H) 3.48 (s, 3 H) |
| | 5.51 (s, 2 H) 7.36-7.46 (m, 1 H) 7.51 (t, J = 7.50 Hz, 1 H) 7.56- |
| | 7.63 (m, 1 H) 7.65 (d, J = 7.75 Hz, 1 H) |
| 26 | ¹H NMR (CDCl₃) δ ppm 1.12-1.27 (m, 2 H) 1.40-1.53 (m, 1 |
| | H) 1.53-1.65 (m, 4 H) 1.91-2.07 (m, 2 H) 3.32 (d, J = 7.00 Hz, |
| | 2 H) 3.37 (s, 3 H) 3.83 (s, 2 H) 6.99-7.08 (m, 2 H) 7.10-7.16 |
| | (m, 1 H) 7.20-7.26 (m, 1 H) |
| 27 | ¹H NMR (CDCl₃) δ ppm 0.73-0.92 (m, 2 H) 0.98-1.12 (m, 3 |
| | H) 1.45-1.59 (m, 4 H) 1.59-1.67 (m, 2 H) 3.24 (d, J = 7.13 Hz, |
| | 2 H) 3.37 (s, 3 H) 3.83 (s, 2 H) 6.98-7.07 (m, 2 H) 7.08-7.15 |
| | (m, 1 H) 7.17-7.25 (m, 1 H) |
| 28 | ¹H NMR (CDCl₃) δ ppm 0.94-1.06 (m, 2 H) 1.13-1.25 (m, 3 |
| | H) 1.33-1.72 (br s, 8 H) 3.18 (d, J = 7.88 Hz, 2 H) 3.39-3.42 (m, |
| | 3 H) 4.00 (s, 2 H) 7.14 (d, J = 7.75 Hz, 1 H) 7.27-7.39 (m, 1 H) |
| | 7.39-7.49 (m, 1 H) 7.60-7.71 (m, 1 H) |
| 29 | ¹H NMR (CDCl₃) δ ppm 3.49 (s, 3 H) 3.67 (s, 2 H) 4.78 (s, 2 H) |
| | 6.69-6.82 (m, 2 H) 6.96 (dd, J = 8.44, 5.32 Hz, 2 H) 7.37 (d, |
| | J = 8.38 Hz, 1 H) 7.83 (d, J = 1.38 Hz, 1 H) 8.16 (dd, J = 8.38, 1.88 |
| | Hz, 1 H) 8.55 (s, 2 H) |
| 30 | ¹H NMR (CDCl₃) δ ppm 0.82 (s, 6 H) 2.53-2.62 (m, 4 H) 3.25 |
| | (s, 2 H) 3.28 (s, 3 H) 3.63 (br t, J = 5.32 Hz, 2 H) 3.67-3.74 (m, |
| | 2 H) 4.04 (s, 2 H) 6.94-7.02 (m, 1 H) 7.16-7.23 (m, 2 H) |
| 31 | ¹H NMR (CDCl₃) δ ppm 0.73-0.90 (m, 2 H) 0.99-1.15 (m, 3 |
| | H) 1.32-1.51 (m, 1 H) 1.51-1.65 (m, 5 H) 2.13 (d, J = 7.25 Hz, |
| | 2 H) 2.53 (s, 3 H) 3.43 (s, 3 H) 4.76 (s, 2 H) 7.03-7.11 (m, 1 H) |
| | 7.11-7.16 (m, 1 H) 8.38 (d, J = 4.00 Hz, 1 H) |
| 32 | ¹H NMR (CDCl₃) δ ppm 0.76 (s, 3 H) 0.89 (s, 3 H) 0.99-1.13 |
| | (m, 2 H) 1.16-1.30 (m, 3 H) 1.35 (m, 2 H) 1.53-1.74 (m, 2 H) |
| | 3.22 (dd, J = 14.01, 11.51 Hz, 1 H) 3.37 (s, 3 H) 3.47-3.56 (m, 1 |
| | H) 3.78-3.91 (m, 2 H) 6.96-7.13 (m, 3 H) 7.18-7.27 (m, 1 H) |
| 33 | ¹H NMR (CDCl₃) δ ppm 0.85 (s, 3 H) 1.01 (s, 3 H) 1.07-1.23 |
| | (m, 2 H) 1.28-1.46 (m, 4 H) 1.49-1.59 (m, 2 H) 1.62-1.70 (m, |
| | 1 H) 3.28 (s, 3 H) 3.37 (dd, J = 14.13, 11.38 Hz, 1 H) 3.73 (dd, |
| | J = 14.07, 3.44 Hz, 1 H) 3.86-3.98 (m, 2 H) 6.93-7.02 (m, 1 H) |
| | 7.14-7.25 (m, 2 H) |
| 34 | ¹H NMR (CDCl₃) δ ppm 1.17 (t, J = 7.19 Hz, 3 H) 1.23-1.33 (m, |
| | 2 H) 1.52-1.85 (m, 4 H) 1.95-2.12 (m, 2 H) 3.45 (d, J = 7.38 Hz, |
| | 2 H) 3.69 (q, J = 7.25 Hz, 2 H) 3.93 (d, J = 1.25 Hz, 2 H) 6.91-7.05 |
| | (m, 1 H) 7.14-7.28 (m, 2 H) |
| 35 | ¹H NMR (CDCl₃) δ ppm −0.08-0.03 (m, 2 H) 0.05-0.13 (m, 2 |
| | H) 0.64-0.72 (m, 2 H) 0.87-1.02 (m, 2 H) 1.35-1.56 (m, 5 H) |
| | 3.21 (d, J = 7.38 Hz, 2 H) 3.26 (s, 3 H) 3.79 (s, 2 H) 6.80 (td, |
| | J = 8.25, 2.63 Hz, 1 H) 6.97-7.05 (m, 2 H) |
| 36 | ¹H NMR (CDCl₃) δ ppm 1.08-1.21 (m, 2 H) 1.21-1.30 (m, 1 |
| | H) 1.32-1.39 (m, 2 H) 1.49-1.63 (m, 1 H) 1.63-1.73 (m, 3 H) |
| | 1.91-2.03 (m, 2 H) 3.37 (s, 3 H) 3.41-3.50 (m, 2 H) 3.89 (s, 2 |
| | H) 6.91 (td, J = 8.22, 2.56 Hz, 1 H) 7.07-7.16 (m, 2 H) |
| 37 | ¹H NMR (CDCl₃) δ ppm 0.98-1.10 (m, 2 H) 1.16-1.32 (m, 2 |
| | H) 1.32-1.64 (br s, 7 H) 1.64-1.78 (m, 2 H) 3.24 (d, J = 7.75 Hz, |
| | 2 H) 3.38 (s, 3 H) 3.84 (s, 2 H) 6.97-7.09 (m, 2 H) 7.09-7.14 |
| | (m, 1 H) 7.17-7.25 (m, 1 H) |
| 38 | ¹H NMR (CDCl₃) δ ppm 0.71-0.91 (m, 2 H) 0.94-1.08 (m, 2 |
| | H) 1.39-1.67 (m, 7 H) 2.20 (d, J = 7.25 Hz, 2 H) 3.41-3.51 (m, |
| | 3 H) 4.94 (s, 2 H) 7.43 (d, J = 8.38 Hz, 1 H) 8.03 (d, J = 1.75 Hz, 1 |
| | H) 8.21 (dd, J = 8.44, 2.06 Hz, 1 H) 8.53 (s, 2 H) |
| 39 | ¹H NMR (CDCl₃) δ ppm 1.01-1.14 (m, 2 H) 1.24-1.33 (m, 2 |
| | H) 1.48-1.65 (m, 5 H) 1.84-2.00 (m, 2 H) 3.33-3.45 (br s, 5 |
| | H) 4.00 (s, 2 H) 7.15 (d, J = 7.75 Hz, 1 H) 7.31-7.40 (m, 1 H) |
| | 7.41-7.50 (m, 1 H) 7.65 (d, J = 7.75 Hz, 1 H) |
| 40 | ¹H NMR (CDCl₃) δ ppm 0.77-0.93 (m, 1 H) 0.97-1.12 (m, 1 |
| | H) 1.31-1.42 (m, 2 H) 1.53-1.65 (m, 4 H) 1.73-1.87 (m, 2 H) |
| | 3.21 (d, J = 7.50 Hz, 1 H) 3.34 (d, J = 7.88 Hz, 1 H) 3.41 (d, J = 3.00 |
| | Hz, 3 H) 3.99 (s, 2 H) 7.15 (br d, J = 7.63 Hz, 1 H) 7.30-7.40 (m, |
| | 1 H) 7.40-7.49 (m, 1 H) 7.65 (br d, J = 7.75 Hz, 1 H) |
| 41 | ¹H NMR (CDCl₃) δ ppm 0.76 (s, 3 H) 0.80 (s, 3 H) 0.92-1.15 |
| | (m, 5 H) 1.21-1.36 (m, 4 H) 1.41 (br dd, J = 11.69, 2.69 Hz, 2 H) |
| | 3.36 (s, 3 H) 3.39-3.50 (m, 2 H) 3.89 (s, 2 H) 6.90 (td, J = 8.25, |
| | 2.63 Hz, 1 H) 7.06-7.16 (m, 2 H) |
| 42 | ¹H NMR (CDCl₃) δ ppm 1.09-1.28 (m, 2 H) 1.41-1.53 (m, 1 |
| | H) 1.57 (br dd, J = 10.63, 2.38 Hz, 4 H) 1.92-2.06 (m, 2 H) 3.30 |
| | (d, J = 7.00 Hz, 2 H) 3.38 (s, 3 H) 3.95 (s, 2 H) 7.10-7.24 (m, 3 |
| | H) 7.31-7.41 (m, 1 H) |
| 43 | ¹H NMR (CDCl₃) δ ppm 0.16-0.30 (m, 2 H) 0.35-0.49 (m, 2 |
| | H) 0.99-1.17 (m, 1 H) 1.17-1.36 (m, 2 H) 1.50-1.64 (m, 1 H) |
| | 1.64-1.82 (m, 4 H) 1.96-2.14 (m, 2 H) 3.45 (d, J = 7.38 Hz, 2 H) |
| | 3.51 (d, J = 7.00 Hz, 2 H) 3.94 (d, J = 1.13 Hz, 2 H) 6.92-7.02 (m, |
| | 1 H) 7.12-7.29 (m, 2 H) |
| 44 | ¹H NMR (CDCl₃) δ ppm 1.30-1.48 (m, 2 H) 1.57-1.80 (m, 4 |
| | H) 1.83 (td, J = 7.38, 3.88 Hz, 1 H) 2.08-2.22 (m, 2 H) 3.56 (d, |
| | J = 7.38 Hz, 2 H) 4.05 (d, J = 1.13 Hz, 2 H) 4.32 (q, J = 8.38 Hz, 2 |
| | H) 7.03-7.13 (m, 1 H) 7.22-7.38 (m, 2 H) |
| 45 | ¹H NMR (CDCl₃) δ ppm 0.76 (s, 3 H) 0.80 (s, 3 H) 0.91-1.12 |
| | (m, 5 H) 1.22-1.34 (m, 4 H) 1.38 (br dd, J = 11.82, 2.56 Hz, 2 H) |
| | 3.37 (s, 3 H) 3.39-3.48 (m, 2 H) 3.84 (s, 2 H) 6.99-7.07 (m, 2 |
| | H) 7.07-7.15 (m, 1 H) 7.15-7.26 (m, 1 H) |
| 46 | ¹H NMR (CDCl₃) δ ppm 1.05-1.19 (m, 2 H) 1.24-1.37 (m, 2 |
| | H) 1.52-1.70 (br s, 5 H) 1.91-2.02 (m, 2 H) 3.38 (s, 3 H) 3.41- |
| | 3.52 (m, 2 H) 3.84 (s, 2 H) 6.96-7.10 (m, 2 H) 7.10-7.16 (m, |
| | 1 H) 7.16-7.27 (m, 1 H) |
| 47 | ¹H NMR (CDCl₃) δ ppm −0.07-0.04 (m, 2 H) 0.04-0.16 (m, 2 |
| | H) 0.67 (br d, J = 14.63 Hz, 2 H) 0.88-1.03 (m, 2 H) 1.35-1.51 |
| | (m, 5 H) 3.21 (d, J = 7.25 Hz, 2 H) 3.28 (s, 3 H) 3.75 (s, 2 H) 6.89- |
| | 6.97 (m, 2 H) 6.99-7.06 (m, 1 H) 7.08-7.16 (m, 1 H) |
| 48 | ¹H NMR (CDCl₃) δ ppm −0.15-−0.03 (m, 2 H) −0.03-0.08 (m, |
| | 2 H) 0.60 (br d, J = 14.01 Hz, 2 H) 0.78-0.95 (m, 2 H) 1.24-1.41 |
| | (m, 5 H) 3.09 (d, J = 7.25 Hz, 2 H) 3.25 (s, 3 H) 3.86 (s, 2 H) 7.00 |
| | (d, J = 7.75 Hz, 1 H) 7.14-7.25 (m, 1 H) 7.25-7.35 (m, 1 H) 7.49 |
| | (d, J = 7.75 Hz, 1 H) |
| 49 | ¹H NMR (CDCl₃) δ ppm 0.73 (s, 3 H) 0.78 (s, 3 H) 0.87-0.96 |
| | (m, 2 H) 0.96-1.11 (m, 3 H) 1.17-1.41 (m, 6 H) 3.34-3.39 (m, |
| | 2 H) 3.39 (s, 3 H) 4.00 (s, 2 H) 7.13 (d, J = 7.63 Hz, 1 H) 7.29- |
| | 7.40 (m, 1 H) 7.40-7.49 (m, 1 H) 7.64 (d, J = 7.75 Hz, 1 H) |
| 50 | ¹H NMR (CDCl₃) δ ppm 1.12-1.36 (m, 2 H) 1.44-1.76 (m, 5 |
| | H) 1.93-2.07 (m, 2 H) 3.44 (d, J = 7.38 Hz, 2 H) 3.93 (s, 2 H) |
| | 4.82 (s, 2 H) 6.91-7.01 (m, 1 H) 7.13-7.30 (m, 7 H) |
| 51 | ¹H NMR (CDCl₃) δ ppm 0.91-1.06 (m, 1 H) 1.11-1.26 (m, 1 |
| | H) 1.40-1.55 (m, 2 H) 1.58-1.69 (m, 1 H) 1.69-1.77 (m, 2 H) |
| | 1.87-1.98 (m, 2 H) 3.35 (d, J = 7.50 Hz, 2 H) 3.44-3.48 (br s, 4 |
| | H) 3.90 (s, 2 H) 7.06-7.16 (m, 2 H) 7.17-7.23 (m, 1 H) 7.25- |
| | 7.34 (m, 1 H) |
| 52 | ¹H NMR (CDCl₃) δ ppm 0.87-1.04 (m, 1 H) 1.04-1.26 (m, 1 |
| | H) 1.37-1.48 (m, 1 H) 1.48-1.77 (m, 5 H) 1.77-1.94 (m, 2 H) |
| | 3.24-3.30 (m, 1 H) 3.37 (m, 3 H) 3.40 (d, J = 7.75 Hz, 1 H) 3.88 |
| | (s, 2 H) 6.84-6.97 (m, 1 H) 7.08-7.15 (m, 2 H) |
| 53 | ¹H NMR (CDCl₃) δ ppm 3.26 (s, 3 H) 3.74-3.78 (br s, 4 H) 34.00 |
| | (s, 2 H) 6.96 (br t, J = 8.17 Hz, 1 H) 7.13-7.22 (m, 2 H) |
| 54 | ¹H NMR (CDCl₃) δ ppm 1.13 (dtd, J = 13.51, 10.18, 10.18, 3.00 |
| | Hz, 2 H) 1.24-1.37 (m, 3 H) 1.40-1.59 (m, 5 H) 1.59-1.67 (m, |
| | 2 H) 1.70-1.79 (m, 2 H) 3.31 (d, J = 7.81 Hz, 2 H) 4.06 (s, 2 H) |
| | 4.38 (q, J = 8.36 Hz, 2 H) 7.16-7.20 (m, 1 H) 7.23-7.28 (m, 2 |
| | H) 7.41-7.46 (m, 1 H) |
| 55 | ¹H NMR (CDCl₃) δ ppm 1.12 (dtd, J = 13.46, 10.16, 10.16, 2.91 |
| | Hz, 2 H) 1.27-1.35 (m, 2 H) 1.42-1.54 (m, 4 H) 1.55-1.60 (m, |
| | 2 H) 1.60-1.66 (m, 2 H) 1.71-1.80 (m, 1 H) 3.30 (d, J = 7.81 Hz, |
| | 2 H) 3.44-3.49 (m, 3 H) 3.99-4.06 (m, 2 H) 7.17-7.21 (m, 1 |
| | H) 7.22-7.25 (m, 2 H) 7.40-7.44 (m, 1 H) |
| 56 | ¹H NMR (CDCl₃) δ ppm 1.03 (s, 3 H) 1.39-1.46 (m, 2 H) 1.49- |
| | 1.55 (m, 8 H) 1.60-1.67 (m, 2 H) 3.33-3.36 (m, 3 H) 3.53 (s, 2 |
| | H) 3.98 (d, J = 1.45 Hz, 2 H) 7.02-7.08 (m, 1 H) 7.22-7.27 (m, |
| | 2 H) |
| 57 | ¹H NMR (CDCl₃) δ ppm 1.28-1.47 (m, 2 H) 1.55-1.82 (br s, 4 |
| | H) 1.84 (br s, 1 H) 2.13 (br s, 2 H) 2.42-2.62 (m, 2 H) 3.54 (br |
| | d, J = 7.25 Hz, 2 H) 3.93-4.04 (br s, 4 H) 7.01-7.13 (m, 1 H) |
| | 7.23-7.33 (m, 2 H) |
| 58 | ¹H NMR (CDCl₃) δ ppm 1.29-1.45 (m, 2 H) 1.62-1.90 (m, 5 |
| | H) 2.06-2.21 (m, 2 H) 3.55 (br d, J = 7.25 Hz, 2 H) 4.01 (s, 2 H) |
| | 4.07 (td, J = 13.48, 4.06 Hz, 2 H) 5.80-6.16 (m, 1 H) 7.07 (br t, |
| | J = 8.25 Hz, 1 H) 7.22-7.33 (m, 2 H) |
| 59 | ¹H NMR (CDCl₃) δ ppm 1.04-1.23 (m, 2 H) 1.23-1.39 (m, 2 |
| | H) 1.39-1.80 (m, 9 H) 2.26 (br d, J = 6.38 Hz, 2 H) 3.43 (s, 3 H) |
| | 5.01 (s, 2 H) 7.03 (br t, J = 8.69 Hz, 1 H) 7.18-7.36 (m, 2 H) |
| 60 | ¹H NMR (CDCl₃) δ ppm 1.11-1.28 (m, 3 H) 1.34 (br t, J = 6.94 |
| | Hz, 3 H) 1.45-1.78 (br s, 10 H) 2.28 (br d, J = 6.50 Hz, 2 H) 3.85 |
| | (q, J = 6.80 Hz, 2 H) 5.05 (s, 2 H) 7.05 (br t, J = 8.76 Hz, 1 H) |
| | 7.24-7.38 (m, 2 H) |
| 61 | ¹H NMR (CDCl₃) δ ppm 1.08-1.24 (m, 2 H) 1.30-1.41 (m, 2 |
| | H) 1.42-1.74 (m, 9 H) 2.28 (br d, J = 6.63 Hz, 2 H) 4.37 (q, J = 8.38 |
| | Hz, 2 H) 5.06 (s, 2 H) 7.06 (br t, J = 8.88 Hz, 1 H) 7.23-7.46 (m, |
| | 2 H) |
| 62 | ¹H NMR (CDCl₃) δ ppm 1.12-1.26 (m, 2 H) 1.39-1.60 (br s, 5 |
| | H) 1.92-2.07 (m, 2 H) 3.31 (d, J = 7.13 Hz, 2 H) 3.99 (s, 2 H) |
| | 4.30 (q, J = 8.38 Hz, 2 H) 7.08-7.15 (m, 1 H) 7.17-7.24 (m, 2 |
| | H) 7.33-7.41 (m, 1 H) |
| 63 | ¹H NMR (CDCl₃)δ ppm 1.13-1.23 (m, 2 H) 1.27 (t, J = 7.19 Hz, |
| | 3 H) 1.39-1.48 (m, 1 H) 1.50-1.65 (m, 4 H) 1.92-2.05 (m, 2 |
| | H) 3.28 (d, J = 7.13 Hz, 2 H) 3.77 (q, J = 7.21 Hz, 2 H) 3.96 (s, 2 |
| | H) 7.08-7.13 (m, 1 H) 7.15-7.22 (m, 2 H) 7.32-7.41 (m, 1 H) |
| 64 | ¹H NMR (CDCl₃)δ ppm 1.05-1.20 (m, 2 H) 1.24-1.33 (m, 2 H) |
| | 1.35 (t, J = 7.19 Hz, 3 H) 1.41-1.70 (br s, 8 H) 1.70-1.81 (m, 1 |
| | H) 3.28 (d, J = 7.88 Hz, 2 H) 3.85 (q, J = 7.13 Hz, 2 H) 4.04 (s, 2 |
| | H) 7.13-7.19 (m, 1 H) 7.21-7.26 (m, 2 H) 7.38-7.46 (m, 1 H) |
| 65 | ¹H NMR (CDCl₃) δ ppm 1.19-1.32 (m, 3 H) 1.55-1.66 (m, 1 |
| | H) 1.84-1.97 (m, 2 H) 2.01-2.12 (m, 1 H) 2.17-2.29 (m, 2 H) |
| | 2.51 (dquin, J = 16.16, 8.09, 8.09, 8.09, 8.09 Hz, 1 H) 3.65 (d, |
| | J = 7.63 Hz, 2 H) 3.77 (q, J = 7.21 Hz, 2 H) 4.02 (s, 2 H) 7.04-7.09 |
| | (m, 1 H) 7.24-7.31 (m, 2 H) |
| 66 | ¹H NMR (CDCl₃) δ ppm 0.28-0.36 (m, 2 H) 0.45-0.52 (m, 2 |
| | H) 1.12-1.18 (m, 1 H) 1.56-1.64 (br s, 1 H) 1.83-1.97 (m, 2 |
| | H) 2.00-2.13 (m, 1 H) 2.16-2.28 (m, 2 H) 2.50 (dt, J = 16.11, |
| | 8.27 Hz, 1 H) 3.60 (d, J = 6.99 Hz, 2 H) 3.66 (d, J = 7.63 Hz, 2 H) |
| | 4.03 (s, 2 H) 7.03-7.09 (m, 1 H) 7.23-7.32 (m, 2 H) |
| 67 | ¹H NMR (CDCl₃) δ ppm 1.18-1.31 (m, 2 H) 1.39-1.49 (m, 2 |
| | H) 1.49-1.65 (m, 4 H) 1.65-1.78 (m, 4 H) 1.94 (br d, J = 7.63 |
| | Hz, 1 H) 3.51 (br d, J = 7.81 Hz, 2 H) 4.05 (s, 2 H) 4.32 (q, J = 8.23 |
| | Hz, 2 H) 7.07 (br t, J = 8.45 Hz, 1 H) 7.22-7.35 (m, 2 H) |
| 68 | ¹H NMR (CDCl₃) δ ppm 1.34-1.45 (m, 1 H) 1.68-1.77 (m, 3 |
| | H) 1.87-1.96 (m, 2 H) 2.57-2.64 (m, 1 H) 2.68 (s, 3 H) 3.07 (br |
| | s, 1 H) 3.21 (br d, J = 11.87 Hz, 1 H) 3.92-4.05 (m, 1 H) 4.10 (s, |
| | 2 H) 4.21-4.31 (m, 3 H) 7.01-7.08 (m, 1 H) 7.21-7.31 (m, 2 H) |
| 69 | ¹H NMR (CDCl₃) δ ppm 0.28-0.33 (m, 2 H) 0.42-0.49 (m, 2 |
| | H) 1.05 (qd, J = 12.79, 2.76 Hz, 1 H) 1.10-1.18 (m, 1 H) 1.22- |
| | 1.31 (m, 1 H) 1.49-1.61 (m, 3 H) 1.63-1.84 (m, 2H) 1.93-2.04 |
| | (m, 2 H) 2.09-2.18 (m, 1 H) 3.57-3.60 (m, 2 H) 3.62 (d, J = 7.63 |
| | Hz, 2 H) 3.97-4.08 (m, 2 H) 7.05 (br t, J = 8.27 Hz, 1 H) 7.18- |
| | 7.34 (m, 2 H) |
| 70 | ¹H NMR (CDCl₃) δ ppm 1.18-1.29 (m, 2 H) 1.50-1.69 (m, 3 |
| | H) 1.73 (br d, J = 15.51 Hz, 2 H) 2.04 (br d, J = 7.25 Hz, 2 H) 2.31 |
| | (d, J = 7.00 Hz, 2 H) 4.36 (q, J = 8.38 Hz, 2 H) 5.04 (s, 2 H) 7.06 |
| | (t, J = 8.88 Hz, 1 H) 7.25-7.40 (m, 2 H) |
| 71 | ¹H NMR (CDCl₃) δ ppm 1.04-1.24 (m, 2 H) 1.40-1.63 (m, 3 |
| | H) 1.67 (br d, J = 13.38 Hz, 2 H) 1.96 (br d, J = 6.88 Hz, 2 H) 2.23 |
| | (d, J = 7.13 Hz, 2 H) 3.36 (s, 3 H) 4.94 (s, 2 H) 6.96 (t, J = 8.88 Hz, |
| | 1 H) 7.13-7.31 (m, 2 H) |

### Pharmacological Examples

Examples of the invention were found to be active on human P2X7 channel assay by automated patch-clamp.

In order to directly monitor the block of P2X7 channel, an electrophysiological assay was developed and implemented on the QPatchl6X automated electrophysiology instrument.

HEK-293 cells expressing the P2X7 channels were cultured in modified EMEM.

72 hours before experiment, 5 million cells were seeded onto T225 flasks. Just before the experiment cells were washed twice, detached from the flask with trypsin-EDTA, re-suspended in the suspension solution and placed on the QPatch 16x.

The compounds (20 mM in a 100% DMSO) stored at −20° C. were prepared the day of the experiment (a first dilution 1:20 in 100% DMSO to prepare a 1 mM stock solution, then a 1 microM solution in external solution+a serial dilution 1:10).

The standard whole-cell voltage clamp experiments were performed at room temperature. From these experiments the multihole technology was used and the data were sampled at 2 KHz.

The intracellular solution contained (mM) 135 CsF, 10 NaCl, 1 EGTA, 10 HEPES, (pH 7.2 with CsOH) whereas the extracellular contained (mM) 145 NaCl, 4 KCl, 0.5 MgCl₂, 1 CaCl₂, 10 HEPES, 10 Glc (pH 7.4 with NaOH).

After establishment of the seal and the passage in the whole cell configuration, the cells were held at −80 mV. The P2XR7 current was evoked by applying 100 microM of BzATP alone (4 times) and then in the presence increasing concentrations of the compound under investigation (1, 10, 100 and 1000 nM).

The pre-incubation periods 5 to 8 contain increasing concentrations of the compound of interest (1, 10, 100 and 1000 nM), as illustrated in FIGURE (application protocol).

FIGURE: application protocol.

The maximal inward current evoked by BzATP in absence or presence of increasing concentrations of the compounds under investigation was measured and normalized. The potential agonist effect was measured as % of control and as IC50 determined fitting the dose-response curves data with the following equation:

Y=100/(1+10\hat{ }((Log IC50−X)*HillSlope))

Where:

X=log of concentration

Y=normalized response, 100% down to 0%, decreasing as X increases.

Log IC₅₀: same log units as X HillSlope: slope factor or HS, untiless.

Here are reported the potency ranges as A, B, C and D, where A is <10 nM; B is 10 nM-100 nM, C is 0.1-1 μM, D is 1-10 μM.

| | human Q-Patch |
|---|---|
| Example | (activity range, see above) |
| 2 | A |
| 3 | B |
| 5 | B |
| 11 | B |
| 12 | A |
| 13 | D |
| 14 | A |
| 15 | B |
| 16 | B |
| 17 | C |
| 18 | B |
| 19 | C |
| 20 | D |
| 21 | D |
| 22 | D |
| 23 | D |
| 24 | C |
| 25 | C |
| 26 | C |
| 27 | D |
| 28 | C |
| 29 | D |
| 30 | D |
| 31 | C |
| 32 | D |
| 33 | B |
| 34 | B |
| 35 | C |
| 36 | C |
| 37 | C |
| 38 | D |
| 39 | C |
| 40 | C |
| 41 | D |
| 42 | B |
| 43 | B |
| 44 | A |
| 45 | D |
| 46 | D |
| 47 | C |
| 48 | C |
| 49 | D |
| 50 | A |
| 51 | C |
| 52 | C |
| 53 | D |
| 55 | C |
| 56 | D |
| 57 | B |
| 58 | B |
| 60 | C |
| 61 | C |
| 62 | A |
| 63 | B |
| 65 | D |
| 66 | C |
| 67 | B |
| 68 | D |
| 69 | B |
| 70 | C |

The Compounds of the Invention were Found to be Active on a Human P2X7 Channel Calcium-Influx Assay

Extracellular binding of Bz-ATP to P2X7 receptor opens the channel and allows Ca²⁺ influx into the cells. This Ca²⁺ entry was measured in HEK-293 cells stably transfected with P2X7 receptor using Screen Quest™ Fluo-8 No Wash Calcium Assay Kit (AAt Bioquest®, cat. 36316). Once inside the cell, the lipophilic blocking groups of Fluo-8 are cleaved by non-specific cell esterases, resulting in a negatively-charged fluorescent dye that stays inside cells. Its fluorescence increases upon binding to calcium. When HEK-293/P2X7 cells are stimulated with Bz-ATP, Ca²⁺ enters the cells and the fluorescence of Fluo-8 NW increases. The dye has an absorption spectrum compatible with excitation at 488 nm by argon laser sources and its emission wavelength is in the range of 515-575 nm.

HEK-293 cells stably transfected with P2X7 receptor were seeded overnight in growth medium at 10,000 to 20,000 cells/well in 384-well plate. 24 hours later, the medium was removed, and the cells were pre-loaded at RT for 1 hour with 20 μL/w of Fluo-8 NW. Then 10 μL/w of test compounds and reference antagonist A438079 at 3×-concentration were injected with the FLIPRTETRA and the kinetic response over a period of five minutes was monitored. A second injection of 15 μL/w of 3× reference activator (Bz-ATP at EC₈₀) was performed with the FLIPR TETRA and the signal of the emitted fluorescence was recorded for additional three minutes. All the experiment was carried out in a Low Divalent Cation Assay Buffer (0.3 mM Ca²⁺ and 0 mM Mg²⁺). The effect of the test compounds was measured as percent inhibition vs the reference antagonist and IC₅₀ values were calculated accordingly. Here are reported the potency ranges as A, B, C and D, where A is <200 nM; B is 200 nM-1 μM, C is 1-10 μM, D is 10-30 μM.

| | humanP2X7 | mouseP2X7 | ratP2X7 |
|---|---|---|---|
| | (activity range, | (activity range, | (activity range, |
| Example | see above) | see above) | see above) |
| 1 | C | C | B |
| 2 | B | B | A |
| 3 | A | B | A |
| 4 | B | C | B |
| 5 | B | C | A |
| 6 | B | B | A |
| 7 | A | B | A |
| 8 | C | C | C |
| 9 | B | B | A |
| 10 | D | C | B |
| 11 | A | B | A |
| 12 | A | A | A |
| 13 | D | D | D |
| 14 | A | A | A |
| 15 | A | C | A |
| 16 | B | C | B |
| 17 | B | C | A |
| 18 | A | C | A |
| 19 | C | D | B |
| 20 | D | D | D |
| 21 | C | D | C |
| 22 | D | D | D |
| 23 | B | B | A |
| 24 | B | C | A |
| 25 | C | D | C |
| 26 | B | C | A |
| 27 | C | C | A |
| 28 | B | C | A |
| 29 | D | D | D |
| 30 | C | D | C |
| 31 | C | D | C |
| 32 | C | C | A |
| 33 | A | B | A |
| 34 | A | A | A |
| 35 | B | C | A |
| 36 | B | C | B |
| 37 | B | B | A |
| 38 | B | C | B |
| 39 | B | C | B |
| 40 | A | C | A |
| 41 | B | D | C |
| 42 | A | B | A |
| 43 | A | A | A |
| 44 | A | A | A |
| 45 | C | D | C |
| 46 | C | C | B |
| 47 | B | C | A |
| 48 | B | C | A |
| 49 | C | D | D |
| 50 | A | A | A |
| 51 | B | C | A |
| 52 | B | C | A |
| 53 | D | D | D |
| 54 | B | B | A |
| 55 | B | C | A |
| 56 | C | D | C |
| 57 | A | A | A |
| 58 | A | A | A |
| 59 | B | B | A |
| 60 | B | C | A |
| 61 | B | C | A |
| 62 | A | A | A |
| 63 | A | B | A |
| 64 | A | B | A |
| 65 | C | D | B |
| 66 | B | C | A |
| 67 | A | A | A |
| 68 | C | C | B |
| 69 | A | B | A |
| 70 | B | C | A |
| 71 | B | C | B |

1. A compound of the following formula (I) or a pharmaceutically acceptable salt thereof: 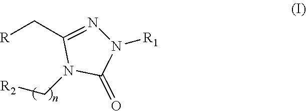
including any stereochemically isomeric form thereof, wherein:
R is an aromatic, aliphatic, heteroaromatic or heteroaliphatic
ring optionally substituted by one or more substituents selected from:
halogen;
C₁-C₄ alkyl optionally substituted by one or more halogen;
R₁ is C₃-C₆ cycloalkyl optionally substituted by one or more halogens or C₁-C₄ alkyl optionally substituted by one or more substituents selected from:
halogen;
OR₃, wherein R₃ is H or C₁-C₄ alkyl optionally substituted by one or more halogens;
NR₄R₅ group, wherein R₄ and R₅ are H or C₁-C₄ alkyl optionally substituted by one or more halogens;
C₃-C₆ cycloalkyl optionally substituted by one or more halogens;
phenyl ring, optionally substituted by halogen;
n is 1 or 2; preferably n is 1;
R₂ is selected from an aromatic, heteroaromatic, aliphatic and heteroaliphatic monocyclic or bicyclic ring optionally substituted by one or more substituents selected from:
halogen;
a C₁-C₄ alkyl optionally substituted by one or more halogens; and
an heteroaromatic ring optionally substituted by one or more halogens;
or R₂ is —OH.

2. A compound of formula (I) according to claim 1 or a pharmaceutically acceptable salt thereof, including any stereochemically isomeric form thereof, wherein
R is phenyl, pyridinyl, cyclohexyl, cycloheptyl, piperidinyl, or piperazinyl optionally substituted by one or more substituents selected from:
halogen, preferably Cl and F;
C₁-C₄ alkyl preferably methyl optionally substituted by one or more halogen, preferably methyl or trifluoromethyl.

3. A compound of formula (I) according to claim 1 or a pharmaceutically acceptable salt thereof, including any stereochemically isomeric form thereof, wherein R₁ is C₁-C₄ alkyl, preferably methyl or ethyl, optionally substituted by:
one or more halogens, preferably F,
C₃-C₄ cycloalkyl, preferably cyclopropyl, optionally substituted by halogen, preferably F, or
a phenyl ring.

4. A compound of formula (I) according to claim 1 or a pharmaceutically acceptable salt thereof, including any stereochemically isomeric form thereof, wherein R₂ is selected from aromatic, aliphatic, heteroaromatic or heteroaliphatic ring selected from phenyl, C₃-C₇ cycloakyl, C₆-C₈ bicycloalkanyl, pyridinyl, piperidinyl, tetrahydrofuranyl and morpholinyl, wherein said rig is optionally substituted by one or more substituents selected from halogen, heteroaromatic ring optionally substituted by halogen, and C₁-C₄ alkyl, optionally substituted by one or more halogen atom(s) or R₂ is —OH.

5. A compound of formula (I) according to claim 1 or a pharmaceutically acceptable salt thereof, including any stereochemically isomeric form thereof, wherein:
R is selected from cycloheptyl, cyclohexyl or phenyl, optionally substituted by one or more halogens, preferably F or Cl, C₁-C₄ alkyl, preferably methyl, optionally substituted by halogen, preferably F;
R₁ is selected from C₁-C₄ alkyl, preferably methyl or ethyl, optionally substituted by halogen, preferably F; or C₃-C₄ cycloalkyl, preferably cyclopropyl, optionally substituted by halogen, preferably F, or a phenyl ring;
n is 1 or 2; preferably n is 1.
R₂ is selected from:
phenyl, optionally substituted by one or more substituents selected from halogen, preferably F or Cl, C₁-C₄ alkyl optionally substituted by one or more halogen, preferably F, and pyrimidinyl optionally substituted by one or more halogen, preferably F;
pyridinyl optionally substituted by one or more C₁-C₄ alkyl, preferably methyl, optionally substituted by one or more halogen, preferably F;
piperidinyl optionally substituted by C₁-C₄ alkyl, preferably methyl;
cyclohexyl optionally substituted by one or more substituents selected from halogen, preferably F, C₁-C₄ alkyl, preferably methyl, optionally substituted by halogen, preferably F;
cyclopropyl, cyclopentyl or cycloheptyl, optionally substituted by one or more halogen, preferably F;
terahydrofuranyl;
morpholinyl optionally substituted by one or more C₁-C₄ alkyl, preferably methyl;
bicyclo[3.1.0]hexan-3-yl;
spiro[2.5]octan-6-yl optionally substituted by one or more halogens, preferably F;
—OH.

6. A compound of formula (I) according to claim 1 or a pharmaceutically acceptable salt thereof, including any stereochemically isomeric form thereof, wherein:
R is independently selected from cyclohexyl, 4,4-difluoro cyclohexyl, cicloheptyl, 2-chloro-6-fluorophenyl, 2-chloro-4-fluorophenyl, 2-fluorophenyl, 4-fluorophenyl, 2-chlorophenyl, 2-trifluoromethylphenyl.
R₁ is independently selected from hydrogen, methyl, ethyl, 2,2,2-trifluoroethyl, cyclopropylmethyl, benzyl, 3,3,3-trifluoropropyl, 2,2-difluoroethyl.
n is 1 or 2; preferably n is 1.
R₂ is independently selected from 4-fluorophenyl, 2-chloro-6-fluorophenyl, 2-trifluoromethylphenyl, 2-chloro-5-(5-fluoropyrimidin-2-yl)phenyl, 2-methylpyridin-3-yl, cyclohexyl, 4,4-difluorocyclohexyl, 3,3-difluorocyclopentyl, 6,6-difluorobicyclo[3.1.0]hexan-3-yl, 4-fluorocyclohexyl, 4-trifluoromethylcyclohexyl, cycloheptyl, 2-(trifluoromethyl)pyridin-4-yl, 4-spiro[2.5]octan-6-yl, 4,4-dimethylcyclohexyl, tetrahydrofuran-2-yl, 1-methylpiperidin-2-yl, morpholinyl, 2,2-dimethylcyclohexyl, 3,3-dimethylmorpholinyl, cycloheptylmethyl and —OH.

7. A compound of formula (I) according to claim 1 selected from the group consisting of:

| Exam- | |
|---|---|
| ple | IUPAC Name |
| 1 | 5-(2-chloro-6-fluorobenzyl)-4-(4-fluorobenzyl)-2-methyl-2,4- |
| | dihydro-3H-1,2,4-triazol-3-one |
| 2 | 5-(2-chloro-6-fluorobenzyl)-4-(cyclohexylmethyl)-2-methyl-2,4- |
| | dihydro-3H-1,2,4-triazol-3-one |
| 3 | 5-(2-chloro-6-fluorobenzyl)-4-((4,4-difluorocyclohexyl)methyl)- |
| | 2-methyl-2,4-dihydro-3H-1,2,4-triazol-3-one |
| 4 | 5-(2-chloro-6-fluorobenzyl)-4-((3,3-difluorocyclopentyl)methyl)- |
| | 2-methyl-2,4-dihydro-3H-1,2,4-triazol-3-one |
| 5 | 5-(2-chloro-6-fluorobenzyl)-4-((6,6-difluorobicyclo[3.1.0]hexan- |
| | 3-yl)methyl)-2-methyl-2,4-dihydro-3H-1,2,4-triazol-3-one |
| 6 | 5-(2-chloro-6-fluorobenzyl)-4-((4-fluorocyclohexyl)methyl)-2- |
| | methyl-2,4-dihydro-3H-1,2,4-triazol-3-one |
| 7 | 5-(2-chloro-6-fluorobenzyl)-2-methyl-4-((4- |
| | (trifluoromethyl)cyclohexyl)methyl)-2,4-dihydro-3H-1,2,4- |
| | triazol-3-one |
| 8 | 5-(2-chloro-4-fluorobenzyl)-4-(4-fluorobenzyl)-2-methyl-2,4- |
| | dihydro-3H-1,2,4-triazol-3-one |
| 9 | 5-(2-chloro-4-fluorobenzyl)-4-(cyclohexylmethyl)-2-methyl-2,4- |
| | dihydro-3H-1,2,4-triazol-3-one |
| 10 | 5-(2-chloro-4-fluorobenzyl)-4-((3,3-difluorocyclopentyl)methyl)- |
| | 2-methyl-2,4-dihydro-3H-1,2,4-triazol-3-one |
| 11 | 5-(2-chloro-4-fluorobenzyl)-4-(cycloheptylmethyl)-2-methyl-2,4- |
| | dihydro-3H-1,2,4-triazol-3-one |
| 12 | 5-(2-chloro-6-fluorobenzyl)-4-(cycloheptylmethyl)-2-methyl-2,4- |
| | dihydro-3H-1,2,4-triazol-3-one |
| 13 | 5-(cyclohexylmethyl)-2-methyl-4-((2-(trifluoromethyl)pyridin-4- |
| | yl)methyl)-2,4-dihydro-3H-1,2,4-triazol-3-one |
| 14 | 5-(2-chloro-6-fluorobenzyl)-2-methyl-4-(spiro[2.5]octan-6- |
| | ylmethyl)-2,4-dihydro-3H-1,2,4-triazol-3-one |
| 15 | 5-(2-chloro-6-fluorobenzyl)-4-(2-(4,4-difluorocyclohexyl)ethyl)- |
| | 2-methyl-2,4-dihydro-3H-1,2,4-triazol-3-one |
| 16 | 5-(2-chloro-6-fluorobenzyl)-4-(2-(4,4-dimethylcyclohexyl)ethyl)- |
| | 2-methyl-2,4-dihydro-3H-1,2,4-triazol-3-one |
| 17 | 4-(cyclohexylmethyl)-2-methyl-5-(2-(trifluoromethyl)benzyl)- |
| | 2,4-dihydro-3H-1,2,4-triazol-3-one |
| 18 | 4-((4,4-difluorocyclohexyl)methyl)-2-methyl-5-(2- |
| | (trifluoromethyl)benzyl)-2,4-dihydro-3H-1,2,4-triazol-3-one |
| 19 | 4-((3,3-difluorocyclopentyl)methyl)-2-methyl-5-(2- |
| | (trifluoromethyl)benzyl)-2,4-dihydro-3H-1,2,4-triazol-3-one |
| 20 | 5-(2-chloro-6-fluorobenzyl)-2-methyl-4-((tetrahydrofuran-2- |
| | yl)methyl)-2,4-dihydro-3H-1,2,4-triazol-3-one |
| 21 | 5-(2-chloro-6-fluorobenzyl)-2-methyl-4-((1-methylpiperidin-2- |
| | yl)methyl)-2,4-dihydro-3H-1,2,4-triazol-3-one |
| 22 | 5-(2-chloro-6-fluorobenzyl)-2-methyl-4-(2-morpholinoethyl)-2,4- |
| | dihydro-3H-1,2,4-triazol-3-one |
| 23 | 4-((2,2-dimethylcyclohexyl)methyl)-2-methyl-5-(2- |
| | (trifluoromethyl)benzyl)-2,4-dihydro-3H-1,2,4-triazol-3-one |
| 24 | 4-(2-chloro-6-fluorobenzyl)-5-(cyclohexylmethyl)-2-methyl-2,4- |
| | dihydro-3H-1,2,4-triazol-3-one |
| 25 | 5-(cyclohexylmethyl)-2-methyl-4-(2-(trifluoromethyl)benzyl)- |
| | 2,4-dihydro-3H-1,2,4-triazol-3-one |
| 26 | 4-((4,4-difluorocyclohexyl)methyl)-5-(2-fluorobenzyl)-2-methyl- |
| | 2,4-dihydro-3H-1,2,4-triazol-3-one |
| 27 | 4-(cyclohexylmethyl)-5-(2-fluorobenzyl)-2-methyl-2,4-dihydro- |
| | 3H-1,2,4-triazol-3-one |
| 28 | 4-(cycloheptylmethyl)-2-methyl-5-(2-(trifluoromethyl)benzyl)- |
| | 2,4-dihydro-3H-1,2,4-triazol-3-one |
| 29 | 4-(2-chloro-5-(5-fluoropyrimidin-2-yl)benzyl)-5-(4- |
| | fluorobenzyl)-2-methyl-2,4-dihydro-3H-1,2,4-triazol-3-one |
| 30 | 5-(2-chloro-6-fluorobenzyl)-4-(2-(3,3- |
| | dimethylmorpholino)ethyl)-2-methyl-2,4-dihydro-3H-1,2,4- |
| | triazol-3-one |
| 31 | 5-(cyclohexylmethyl)-2-methyl-4-((2-methylpyridin-3- |
| | yl)methyl)-2,4-dihydro-3H-1,2,4-triazol-3-one |
| 32 | 4-((2,2-dimethylcyclohexyl)methyl)-5-(2-fluorobenzyl)-2- |
| | methyl-2,4-dihydro-3H-1,2,4-triazol-3-one |
| 33 | 5-(2-chloro-6-fluorobenzyl)-4-((2,2-dimethylcyclohexyl)methyl)- |
| | 2-methyl-2,4-dihydro-3H-1,2,4-triazol-3-one |
| 34 | 5-(2-chloro-6-fluorobenzyl)-4-((4,4-difluorocyclohexyl)methyl)- |
| | 2-ethyl-2,4-dihydro-3H-1,2,4-triazol-3-one |
| 35 | 5-(2-chloro-4-fluorobenzyl)-2-methyl-4-(spiro[2.5]octan-6- |
| | ylmethyl)-2,4-dihydro-3H-1,2,4-triazol-3-one |
| 36 | 5-(2-chloro-4-fluorobenzyl)-4-(2-(4,4-difluorocyclohexyl)ethyl)- |
| | 2-methyl-2,4-dihydro-3H-1,2,4-triazol-3-one |
| 37 | 4-(cycloheptylmethyl)-5-(2-fluorobenzyl)-2-methyl-2,4-dihydro- |
| | 3H-1,2,4-triazol-3-one |
| 38 | 4-(2-chloro-5-(5-fluoropyrimidin-2-yl)benzyl)-5- |
| | (cyclohexylmethyl)-2-methyl-2,4-dihydro-3H-1,2,4-triazol-3-one |
| 39 | 4-(2-(4,4-difluorocyclohexyl)ethyl)-2-methyl-5-(2- |
| | (trifluoromethyl)benzyl)-2,4-dihydro-3H-1,2,4-triazol-3-one |
| 40 | 2-methyl-5-(2-(trifluoromethyl)benzyl)-4-((4- |
| | (trifluoromethyl)cyclohexyl)methyl)-2,4-dihydro-3H-1,2,4- |
| | triazol-3-one |
| 41 | 5-(2-chloro-4-fluorobenzyl)-4-(2-(4,4-dimethylcyclohexyl)ethyl)- |
| | 2-methyl-2,4-dihydro-3H-1,2,4-triazol-3-one |
| 42 | 5-(2-chlorobenzyl)-4-((4,4-difluorocyclohexyl)methyl)-2-methyl- |
| | 2,4-dihydro-3H-1,2,4-triazol-3-one |
| 43 | 5-(2-chloro-6-fluorobenzyl)-2-(cyclopropylmethyl)-4-((4,4- |
| | difluorocyclohexyl)methyl)-2,4-dihydro-3H-1,2,4-triazol-3-one |
| 44 | 5-(2-chloro-6-fluorobenzyl)-4-((4,4-difluorocyclohexyl)methyl)- |
| | 2-(2,2,2-trifluoroethyl)-2,4-dihydro-3H-1,2,4-triazol-3-one |
| 45 | 4-(2-(4,4-dimethylcyclohexyl)ethyl)-5-(2-fluorobenzyl)-2- |
| | methyl-2,4-dihydro-3H-1,2,4-triazol-3-one |
| 46 | 4-(2-(4,4-difluorocyclohexyl)ethyl)-5-(2-fluorobenzyl)-2-methyl- |
| | 2,4-dihydro-3H-1,2,4-triazol-3-one |
| 47 | 5-(2-fluorobenzyl)-2-methyl-4-(spiro[2.5]octan-6-ylmethyl)-2,4- |
| | dihydro-3H-1,2,4-triazol-3-one |
| 48 | 2-methyl-4-(spiro[2.5]octan-6-ylmethyl)-5-(2- |
| | (trifluoromethyl)benzyl)-2,4-dihydro-3H-1,2,4-triazol-3-one |
| 49 | 4-(2-(4,4-dimethylcyclohexyl)ethyl)-2-methyl-5-(2- |
| | (trifluoromethyl)benzyl)-2,4-dihydro-3H-1,2,4-triazol-3-one |
| 50 | 2-benzyl-5-(2-chloro-6-fluorobenzyl)-4-((4,4- |
| | difluorocyclohexyl)methyl)-2,4-dihydro-3H-1,2,4-triazol-3-one |
| 51 | 5-(2-fluorobenzyl)-2-methyl-4-((4- |
| | (trifluoromethyl)cyclohexyl)methyl)-2,4-dihydro-3H-1,2,4- |
| | triazol-3-one |
| 52 | 5-(2-chloro-4-fluorobenzyl)-2-methyl-4-((4- |
| | (trifluoromethyl)cyclohexyl)methyl)-2,4-dihydro-3H-1,2,4- |
| | triazol-3-one |
| 53 | 5-(2-chloro-6-fluorobenzyl)-4-(2-hydroxyethyl)-2-methyl-2,4- |
| | dihydro-3H-1,2,4-triazol-3-one |
| 54 | 5-[(2-chlorophenyl)methyl]-4-(cycloheptylmethyl)-2-(2,2,2- |
| | trifluoroethyl)-2,4-dihydro-3H-1,2,4-triazol-3-one |
| 55 | 5-[(2-chlorophenyl)methyl]-4-(cycloheptylmethyl)-2-methyl-2,4- |
| | dihydro-3H-1,2,4-triazol-3-one |
| 56 | 5-[(2-chloro-6-fluorophenyl)methyl]-2-methyl-4-[(1- |
| | methylcycloheptyl)methyl]-2,4-dihydro-3H-1,2,4-triazol-3-one |
| 57 | 5-[(2-chloro-6-fluorophenyl)methyl]-4-[(4,4- |
| | difluorocyclohexyl)methyl]-2-(3,3,3-trifluoropropyl)-2,4- |
| | dihydro-3H-1,2,4-triazol-3-one |
| 58 | 5-[(2-chloro-6-fluorophenyl)methyl]-4-[(4,4- |
| | difluorocyclohexyl)methyl]-2-(2,2-difluoroethyl)-2,4-dihydro- |
| | 3H-1,2,4-triazol-3-one |
| 59 | 4-[(2-chloro-6-fluorophenyl)methyl]-5-(cycloheptylmethyl)-2- |
| | methyl-2,4-dihydro-3H-1,2,4-triazol-3-one |
| 60 | 4-[(2-chloro-6-fluorophenyl)methyl]-5-(cycloheptylmethyl)-2- |
| | ethyl-2,4-dihydro-3H-1,2,4-triazol-3-one |
| 61 | 4-[(2-chloro-6-fluorophenyl)methyl]-5-(cycloheptylmethyl)-2- |
| | (2,2,2-trifluoroethyl)-2,4-dihydro-3H-1,2,4-triazol-3-one |
| 62 | 5-[(2-chlorophenyl)methyl]-4-[(4,4-difluorocyclohexyl)methyl]- |
| | 2-(2,2,2-trifluoroethyl)-2,4-dihydro-3H-1,2,4-triazol-3-one |
| 63 | 5-[(2-chlorophenyl)methyl]-4-[(4,4-difluorocyclohexyl)methyl]- |
| | 2-ethyl-2,4-dihydro-3H-1,2,4-triazol-3-one |
| 64 | 5-[(2-chlorophenyl)methyl]-4-(cycloheptylmethyl)-2-ethyl-2,4- |
| | dihydro-3H-1,2,4-triazol-3-one |
| 65 | 5-[(2-chloro-6-fluorophenyl)methyl]-4-[(3,3- |
| | difluorocyclopentyl)methyl]-2-ethyl-2,4-dihydro-3H-1,2,4- |
| | triazol-3-one |
| 66 | 5-[(2-chloro-6-fluorophenyl)methyl]-2-(cyclopropylmethyl)-4- |
| | [(3,3-difluorocyclopentyl)methyl]-2,4-dihydro-3H-1,2,4-triazol- |
| | 3-one |
| 67 | 5-[(2-chloro-6-fluorophenyl)methyl]-4-(cycloheptylmethyl)-2- |
| | (2,2,2-trifluoroethyl)-2,4-dihydro-3H-1,2,4-triazol-3-one |
| 68 | 5-[(2-chloro-6-fluorophenyl)methyl]-4-[(1-methylpiperidin-2- |
| | yl)methyl]-2-(2,2,2-trifluoroethyl)-2,4-dihydro-3H-1,2,4-triazol- |
| | 3-one |
| 69 | 5-(2-chloro-6-fluorobenzyl)-2-(cyclopropylmethyl)-4-((4- |
| | (trifluoromethyl)cyclohexyl)methyl)-2,4-dihydro-3H-1,2,4- |
| | triazol-3-one |
| 70 | 4-[(2-chloro-6-fluorophenyl)methyl]-5-[(4,4- |
| | difluorocyclohexyl)methyl]-2-(2,2,2-trifluoroethyl)-2,4-dihydro- |
| | 3H-1,2,4-triazol-3-one |
| 71 | 4-[(2-chloro-6-fluorophenyl)methyl]-5-[(4,4- |
| | difluorocyclohexyl)methyl]-2-methyl-2,4-dihydro-3H-1,2,4- |
| | triazol-3-one |

8. A process for preparing a compound of formula (I) according to claim 1 comprising the reaction of a a compound of formula (II): 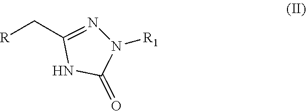
wherein R and R₁ are as defined above, with a compound of formula (III): 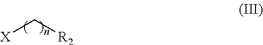
wherein R₂ and n are as defined above, and X is a suitable leaving group; and optionally converting the obtained compound of formula (I) into an addition salt thereof, and/or preparing stereochemically isomeric forms thereof.

9. A pharmaceutical composition comprising a compound of formula (I) or a pharmaceutically acceptable salt thereof, including any stereochemically isomeric form thereof according to claims 1-7 and a pharmaceutically acceptable diluent and/or carrier.

10. A compound of formula (I) or a pharmaceutically acceptable salt thereof, including any stereochemically isomeric form thereof according to claims 1-7 for use as a medicament.

11. A compound of Formula (I) or a pharmaceutically acceptable salt thereof, including any stereochemically isomeric form thereof according to claims 1-7 for use in treatment of conditions or diseases selected from P2X7 receptor mediated conditions or diseases.

12. A compound of formula (I) or a pharmaceutically acceptable salt thereof, including any stereochemically isomeric form thereof according to claims 1-7 for use in prevention and/or treatment of neurodegenerative, cognitive, psychiatric disorders, neuropathic pain, chronic pain, inflammatory processes of the muscular-skeletal system, liver fibrosis, gastrointestinal tract disorders, genito-urinary tract disorders, ophthalmic diseases, Chronic Obstructive Pulmonary Disease (COPD), cancer and proliferative diseases.
